SEQUENCE LISTING (1 sequence)

SEQ ID NO: 1 - protein, 146 aa
  hypothetical: NO
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2005/0118166 A1
(43) Pub. Date: Jun. 2, 2005

(54) Therapeutic applications for the anti-T-BAM (CD40-1) monoclonal antibody 5C8

(75) Inventors: Michael J. Yellin, Montclair, NJ (US); Seth Lederman, New York, NY (US); Leonard Chess, Scarsdale, NY (US); Mihail N. Karpusas, Roslindale, MA (US); David W. Thomas, Wellesley, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Biogen Incorporated

(21) Appl. No.: 10/618,298

(22) Filed: Jul. 11, 2003

Related U.S. Application Data
(63) Continuation of application No. 09/343,001, filed on Jun. 29, 1999, now abandoned.
(63) Continuation of application No. 09/342,314, filed on Jun. 29, 1999, now abandoned.
Application No. 09/343,001 is a division of application No. 08/637,323, filed on Apr. 22, 1996, now Pat. No. 6,340,459.
Application No. 09/342,314 is a division of application No. 08/637,323, filed on Apr. 22, 1996, now Pat. No. 6,340,459.
Application No. 08/637,323 is a continuation-in-part of application No. 08/566,258, filed on Dec. 1, 1995, now abandoned.
Application No. 08/566,258 is a continuation-in-part of application No. 08/567,391, filed on Dec. 1, 1995, now abandoned.

(51) Int. Cl. A61K 39/395

(52) U.S. Cl. 424/141.1; 424/144.1

(57) ABSTRACT

Activation of cells bearing CD40 on their cell surface by CD40 ligand is inhibited by contacting the cells with an agent capable of inhibiting interaction between CD40 ligand and the cells, in an amount effective to inhibit activation of the cells. Activation of cells bearing CD40 on their surface by CD40 ligand in a subject is inhibited by administering to the subject an agent capable of inhibiting interaction between CD40 ligand and the cells, in an amount effective to inhibit activation of the cells. Conditions dependent on CD4O ligand-induced activation of CD40-bearing cells are treated.

This application is a continuation-in-part of U.S. application Ser. Nos. 08/566,258 and 08/567,391, both filed Dec. 1, 1995, the contents of which are hereby incorporated by reference.

Throughout this application, various references are referred to within parentheses. Disclosures of these publications in their entireties are hereby incorporated by reference into this application to more fully describe the state of the art to which this invention pertains. Full bibliographic citation for these references may be found in the text or at the end of this application, preceding the sequence listing and claims.

The invention disclosed herein was made with Government support under NIH Grant Nos. K08-AR-01904, RO1-CA55713, RO1-AI-28367, ROl-AI-14969, HL21006, HL42833, HL50629, and RO1-AI-14969 from the Department of Health and Human Services. Accordingly, the U.S. Government has certain rights in this invention.

### BACKGROUND OF THE INVENTION

CD40 is a 50 kDa cell surface molecule originally described as being expressed on B cells and some epithelial-carcinomas (1,2). CD40 interacts with CD40L (T-BAM, gp39, TRAP), a 30 kDa cell surface molecule transiently expressed on activated CD4+ T cells (3-8). CD40L-CD40 interactions have been extensively studied in the context of T cell-B cell interactions. CD40 ligation plays key roles in B cell activation, proliferation, differentiation, Ig production and rescue from apoptotic signals (9-11). The critical in vivo role of CD40 ligation in B cell differentiation is highlighted by the hyper-IgM syndrome, a humoral immunodeficiency due to mutations in the gene encoding CD40L (12-16). Murine CD40 (17) or CD40L (18) “knockouts” have similar phenotypes to patients with the hyper-IgM syndrome.

Interestingly, recent studies indicate that CD40 expression has a broader cellular distribution than originally described. CD40 has been shown to be expressed on monocytes (19), dendritic cells (22), epithelium (23, 21), basophils (24), and Hodgkin's tumor cells (25). Moreover, various cytokines can regulate CD40 expression on non-B cells. CD40 expression on thymic epithelial cells is upregulated by IL-1α, TNF-α or INF-γ (21). INF-γ, in addition to IL-3 or GM-CSF, similarly upregulates CD40 expression on monocytes (19). Ligation of CD40 in the presence of INF-γand IL-1α stimulates GM-CSF production by thymic epithelial cells (21). In addition, CD40L expressing transfectants induce tumoricidal activity by monocytes and, in the presence of INF-γ, GM-CSF or IL-3, stimulate monocytes to secrete TNF-α, IL-6 or IL-8 (19.).

CD40 is also expressed on cells found within synovial membrane (SM) in patients afflicted with rheumatoid arthritis (RA). An immunohistological survey of cell surface molecules expressed in RA SM found that CD40 was expressed on a variety of cell types, including cells with fibroblast-like morphology (26). In this report it is shown by FACS analysis that CD40 is expressed on cultured synovial membrane (SM) fibroblasts isolated from patients with RA, non-RA inflammatory arthritis (IA) or osteoarthritis (OA). In addition, dermal fibroblasts isolated from normal donors also express CD40. Moreover, CD40 ligation by CD40L⁺ cells induces fibroblast activation and proliferation.

Endothelial cells express surface molecules, such as CD54 (ICAM-1), CD62E (E-selectin) and CD106 (VCAM-1), that mediate adhesive interactions with leukocytes (27-35). The expression of endothelial cell surface adhesion molecules orchestrates recruitment of leukocytes to sites of inflammation and therefore is subject to tight regulation (27, 28). Resting endothelial cells express low levels of CD54 and minimal or no CD62E or CD106. Following activation with IL-1, TNFα, or LPS, endothelial cells rapidly upregulate CD54, CD62E and CD106 expression (27, 28). CD4⁺ T cells may contribute to upregulation of endothelial cell surface adhesion molecules by inducing endothelial cells or other target cells to secrete IL-1 or TNFα (36). However, the molecular details involved in CD4⁺ T cell-endothelial cell interactions that induce endothelial cell activation have not been completely delineated.

It can now be reported that normal human endothelial cells also express CD40 in situ and CD40L-CD40 interactions induce endothelial cell activation in vitro. Frozen sections from normal spleen, thyroid, skin, muscle, kidney, lung or umbilical cord were studied for CD40 expression by immunohistochemistry. Endothelial cells from all tissues studied express CD40 in situ. Moreover, human umbilical vein endothelial cells (HUVEC) express CD40 in vitro and rIFN-γ induces HUVEC CD40 upregulation. CD40 expression on HUVEC is functionally significant because CD40L⁺ Jurkat T cells upregulate HUVEC CD54 (ICAM-1), CD62E (E-selectin) and CD106 (VCAM-1) expression in vitro in a manner inhibited by anti-CD40L mAb 5C8. Additionally, CD40L expressing 293 kidney cell transfectants, but not control transfectants, also upregulate CD54, CD62E and CD106 expression on HUVEC. These results demonstrate that CD40L-CD40 interactions induce endothelial cell activation in vitro. It is shown for the first time that CD40L expressed on the surface of T cells induces activation of CD40+ endothelial cells and that this activation is inhibited by an anti-CD40L monoclonal antibody. Moreover, these results demonstrate a mechanism by which activated CD4⁺ T cells augment inflammatory responses in vivo by upregulating the expression of endothelial cell surface adhesion molecules.

### SUMMARY OF THE INVENTION

This invention provides a method of inhibiting activation by CD40 ligand of cells bearing CD40 on the cell surface, comprising contacting the cells with an agent capable of inhibiting interaction between CD40 ligand and the cells, in an amount effective to inhibit activation of the cells.

This invention provides a method of inhibiting activation by CD40 ligand of cells bearing CD40 on the cell surface, in a subject, comprising administering to the subject an agent capable of inhibiting interaction between CD40 ligand and the cells, in an amount effective to inhibit activation of the cells in the subject.

### DESCRIPTION OF THE FIGURES

FIG. 1. CD40 expression on SM fibroblasts. Shown are FACS analyses of CD40, CD14, CD45 or MHC Class II expression, as indicated, on representative RA or OA SM adherent cells following the first passage in vitro. The X-axis represents mean fluorescence intensity (MFI) and the Y-axis represents cell number. For RA cells, the MFI of CD40 expression or isotype control mAb was 21 and 9, respectively. For OA cells, the MFI of CD40 expression or isotype control mAb was 33 and 9, respectively.

FIG. 2. CD40 expression on resting or rINF-γ stimulated dermal fibroblasts. Shown are FACS analyses of CD40, CD54 or control mAb staining, as indicated, on 3 dermal fibroblast lines. The cells were cultured in the presence or absence of rINF-γ (1000 U/ml) for 24 hours. SK.1 and SK.2 were studied following the second passage and CCD 965 SK was studied following the third passage in culture. The X-axis represents mean fluorescence intensity (MFI) and the Y-axis represents cell number. The number in the upper right hand corner of each graph indicates CD40 MFI (background subtracted).

FIG. 3. Cytokine regulation of SM fibroblast CD40 expression. Shown is a bar graph representing CD40 mean fluorescence intensity (MFI) on a SM fibroblast line (OA.3) following co-culture with rINF-γ (1000 U/ml), rIL-1α (10 pg/ml), rTNF-α (200 U/ml) or combinations of cytokines, as indicated. CD40 expression was determined by FACS analysis and background staining with a control mAb is subtracted for each value. The experiment shown is representative of 3 similar experiments performed.

FIG. 4. Effect of CD40L-CD40 interactions on SM fibroblast CD54 (ICAM-1) expression. Shown are two-color contour graphs demonstrating CD13 expression (X-axis) or CD54 expression (Y-axis) on IA.1 SM fibroblasts cultured 24 hours with media, rINF-γ (1000 U/mi), CD40L⁻ Jurkat B2.7 cells or CD40L⁺ Jurkat D1.1 cells in the presence or absence of anti-CD40L mAb 5C8 or control mAb P1.17. The number in the upper right hand corner of each graph represents CD54 mean fluorescence intensity (MFI). The background MFI of an isotype control mAb is subtracted from each value. The experiment shown is representative of 3 similar experiments performed.

FIG. 5. Transfection of CD40L confers the capacity to upregulate SM fibroblast CD54 (ICAM-1) and CD106 (VCAM-1) expression. Shown are bar graphs indicating CD54 or CD106 MFI on SM fibroblasts following culture for 24 hours with media, CD40L⁺ D1.1 cells, CD40L⁻ B2.7 cells or CD40L⁺ B2.7 transfectants, as indicated. CD54 and CD106 expression were determined by two-color FACS analysis as in FIG. 4. The background MFI of an isotype control mAb is subtracted from each value. The experiment shown is representative of 2 similar experiments performed.

FIG. 6A. Effect of CD40L-CD40 interactions on fibroblast IL-6 secretion. Shown are bar graphs indicating ³H-thymidine incorporation by the IL-6 indicator cell line B9 following the additions of supernatants (final dilution 1:60) from SM fibroblasts cultured with media alone, CD40L⁺ D1.1 cells in the presence or absence of anti-CD40L mAb 5C8 or control mAb P1.17, CD40L⁻ B2.7 cells or CD40L⁺ B2.7 transfectants. The proliferative responses of B9 cells cultured with control supernatants from D1.1 cells, B2.7 cells or CD40L⁺ B2.7 transfectants were 1136 cpm (±113), 2398 cpm (±263) and 1131 cpm (±56). Similar results were obtained with 3 additional SM fibroblast lines.

FIG. 6B. B9 proliferation in response to rIL-6. In a parallel experiment to that shown in FIG. 6A, B9 cells were cultured with varying concentrations of rIL-6.

FIG. 7. Effect of CD40 ligation on SM fibroblast proliferation. Shown are bar graphs from 2 separate experiments demonstrating SM fibroblast ³H-thymidine incorporation following coculture in 1% FM with mitbmycin-C treated CD40L⁻ Jurkat B2.7 cells or CD40L⁺ Jurkat B2.7 transfectants for 48 hours. Where indicated, CD40L⁺ Jurkat B2.7 transfectants were pretreated with anti-CD40L mAb 5C8 (5 μg/ml) or P1.17 control mAb (5 μg/ml) prior to the addition to fibroblasts. In the experiment studying RA.5 proliferation, the proliferation of CD40L⁻ Jurkat B2.7 cells or CD40L⁺ Jurkat B2.7 transfectants was 51±7 cpm and 39±3 cpm, respectively. In the experiment studying OA.6 proliferation, the proliferation of CD40L⁻ Jurkat B2.7 cells or CD40L⁺ Jurkat B2.7 transfectants was 243±5 cpm and 453±95 cpm, respectively. Background proliferation is subtracted in coculture experiments. Also shown are the proliferative responses of fibroblasts following culture in 1% FM or 10% FM. Similar results were obtained in 3 additional experiments. Error bars show observed error.

FIG. 8. Effect of rINF-γ on CD40L mediated SM fibroblast proliferation. Shown are bar graphs demonstrating SM fibroblast ³H-thymidine incorporation following coculture in 1% FM with mitomycin-C treated CD40L⁻ Jurkat B2.7 cells or CD40L⁺ Jurkat B2.7 transfectants for 48 hours. Where indicated, SM fibroblasts were pretreated for 18 hours with rINF-γ (1000 U/ml) prior to the addition of mitomycin-C treated CD40L⁻ B2.7 cells or CD40L⁺ B2.7 transfectants. SM fibroblast proliferation was determined as outlined in Materials and Methods for First Series of Experiments. Background proliferation of CD40L⁻ Jurkat B2.7 cells and CD40L⁺ Jurkat B2.7 transfectants was 185±66 cpm and 65±5 cpm, respectively. Background proliferation is subtracted in coculture experiments. Also shown are the proliferative responses of fibroblasts following culture in 1% FM or 10% FM. Similar results were obtained in 2 additional experiments. Error bars show observed error.

FIGS. 9A-D. Endothelial cells in skin express CD40 in situ. Shown are immunohistologic studies of frozen sections demonstrating the expression of: (a) CD40, skin (magnification 40×), (b) CD34, skin (magnification 40×), (c) CD21, skin (magnification 40×) and (d) control mouse IgG, skin (magnification 40×).

FIGS. 10A-D. Endothelial cells in muscle express CD40, in situ. Shown are immunohistologic studies of frozen sections demonstrating the expression of:(a) CD40, muscle (magnification 40×), (b) CD34, muscle (magnification 40×), (c) CD21, muscle (magnification 40×) and (d) control mouse. IgG, muscle (magnification 40×).

FIG. 11. Endothelial cells in spleen express CD40 in situ. Shown are immunohistologic studies of frozen sections demonstrating the expression of: (a) CD40, spleen (magnification 10×) and (b) control mouse IgG, spleen (magnification 10×).

FIG. 12. Expression of CD40 on HUVEC cells in vitro. Shown are overlapping FACS analysis of CD14, CD46, CD45 or isotype control expression on HUVEC following the first passage. The mean fluorescence intensity of CD14, CD40, CD45 or isotype control expression is 7, 24, 5 and 9, respectively. Shown is representative of CD40 expression on HUVEC isolated from 15 umbilical cords.

FIG. 13. Effect of CD40L-CD40 interactions on HUVEC CD54 (ICAM-1) expression. Shown are two-color contour graphs demonstrating the effects on HUVEC CD54 expression following culture with media, CD40L⁺ Jurkat D1.1 cells or CD40L⁻ Jurkat B2.7 cells for 6 hours. Where indicated, CD40L⁺ D1.1 cells were pretreated with anti-CD40L mAb 5C8 or isotype control mAb P1.17. The X-axis demonstrates CD13 expression and the Y-axis demonstrates CD54 expression. The numbers in the upper right hand corner of each graph indicates percentage of CD13⁺ cells expressing CD54 (background staining of control mAb is subtracted for each value). Shown is representative of 3 similar experiments with different HUVEC lines.

FIG. 14. Effect of CD40L-CD40 interactions on HUVEC CD54, (ICAM-1), CD62E (E-selectin) and CD106 (VCAM-1) expression. Shown are bar graphs representing the percentage of HUVEC expressing CD54, CD62E or CD106 following culture for 6 hours with media, rIL-1α, CD40L⁺ Jurkat D1.1 cells or CD40L⁻ Jurkat B2.7 cells. Where indicated, CD40L⁺ D1.1 cells were pretreated with anti-CD40L mAb 5C8 or isotype control mAb P1.17. HUVEC CD54, CD62E and CD106 expression was determined by two-color FACS analysis as shown in FIG. 3. Background staining of control mAb is subtracted for each value. Shown is representative of 3 similar experiments with different HUVEC lines.

FIG. 15. Effect of CD40L expressing 293 kidney cell transfectants on HUVEC CD54, CD62E and CD106 expression. Shown are two-color contour graphs demonstrating the effects on HUVEC CD54, CD62E and CD106 expression following culture with media, CD40L⁺ Jurkat D1.1 cells, CD8⁺ 293 kidney cell transfectants or CD40L⁺ 293 kidney cell transfectants for 6 hours. The X-axis demonstrates UEA-1 expression and the Y-axis demonstrates CD54 (left panel), CD106 (middle panel) or CD62E (right panel) expression. The numbers in the upper right hand corner of each graph indicates the percentage of UEA-1³⁰ cells expressing CD54, CD106 or CD62E, as indicated (background staining of control mAb is subtracted for each value). Shown is representative of 3 similar experiments with different HUVEC lines.

FIG. 16A. Kinetic analysis of CD40L induced HUVEC CD54, CD62E and CD106 upregulation. Shown are the percentage of HUVEC expressing CD54, CD62E, or CD106 following culture with CD40L⁺ Jurkat D1.1 cells for 6 or 24 hours. The percentage of HUVEC expressing CD54, CD62E or CD106 was determined by two-color FACS analysis (background staining of control mAb is subtracted for each value). Shown is representative of 3 similar experiments with different HUVEC lines.

FIG. 16B. Same as FIG. 16A except that HUVEC were cultured with CD40L-Jurkat B2.7 cells.

FIGS. 17A-Y: Atomic coordinates of crystal structure of soluble extracellular fragment of human CD40L containing residues Gly116-Leu261 SEQ ID NO:1 (in Brookhaven Protein Data Bank format).

### DETAILED DESCRIPTION

This invention provides a method of inhibiting activation by CD40 ligand of cells bearing CD40 on the dell surface, comprising contacting the cells with an agent capable of inhibiting interaction between CD40 ligand and the cells, in an amount effective to inhibit activation of the cells. In one embodiment, the cells bearing CD40 on the cell surface are cells other than B cells. In another embodiment, they are plasma cells, including differentiated plasma cells such as myeloma cells.

This method may be used to inhibit activation of CD40-bearing cells either in vivo or ex vivo. “Interaction between CD40 ligand and CD40 on the cells” refers to one or more aspects, functional or structural, of a CD40-CD40 ligand interrelationship. Therefore, in one embodiment, an agent which inhibits interaction may competitively bind to CD40 ligand in such a way to block or diminish the binding of CD40 ligand to cellular CD40. In another embodiment an agent which inhibits interaction may associate with CD40 or CD40 ligand in a manner which does not inhibit binding of CD40 ligand to cellular CD40, but which influences the cellular response to the CD40 ligation such as by altering the turnover rate of the cellular CD40 or the CD40-agent complex, by altering binding kinetics of CD40 with CD40 ligand, or by altering the rate or extent of cellular activation in response to CD40 ligation.

In specific embodiments of this invention, the non-B cell, CD40-bearing cells are fibroblasts, endothelial cells, epithelial cells, T cells, basophils, macrophages, Reed-Steinberg cells, or dendritic cells. In a more specific embodiment the epithelial cells are keratinocytes. In another embodiment, the macrophages are foam cells (lipid-laden macrophages). Foam cells play a role in autoimmune diseases, for example rheumatoid arthritis and atherosclerosis.

In an embodiment of this invention the agent inhibits binding of CD40 ligand to CD40 on the cells.

In an embodiment of this method, the agent is a protein. In a more specific embodiment, the protein comprises an antibody or portion thereof, for example a Fab, F(ab′)₂, complementarity determining region (CDR) light and/or heavy chain, antibody variable region light and/or heavy chain, or a portion thereof capable of specifically binding to CD40 ligand or CD40 ligand cell-surface receptor. The antibody can be a monoclonal or polyclonal antibody. In embodiments of this invention, the monoclonal antibody is a chimeric antibody, a humanized antibody, or a primatized antibody. In another embodiment the portion of the antibody comprises a single chain antibody. A single chain antibody is made up of variable regions linked by protein spacers in a single protein chain.

In an embodiment of the above-described method, the agent specifically binds to the antigen to which monoclonal antibody 5c8 specifically binds. In a specific embodiment, the agent is monoclonal antibody 5c8.

Monoclonal antibody 5c8 is produced by a hybridoma cell which was deposited on Nov. 14, 1991 with the American Type Culture Collection (ATCC), 10801 University Blvd., Manassas, Va. 20110-2209 under the provisions of the Budapest Treaty for the International Recognition of the Deposit of Microorganisms for the Purposes of Patent Procedure. The hybridoma was accorded ATCC Accession Number HB 10916.

In another embodiment, the antibody specifically binds to CD40. One example of an anti-CD40 antibody is the monoclonal mouse anti-human CD40, available from Genzyme Customer Service (Product 80-3702-01, Cambridge, Mass.). In other embodiments the monoclonal antibody is a chimeric antibody, a primatized antibody, a humanized antibody, or an antibody which includes a CDR region from a first human and an antibody scaffold from a second human.

In one embodiment of this invention the protein is soluble, monomeric CD40-L protein, comprising all or part of the extracellular region of CD40-L, or variant thereof. The extracellular region of CD40-L contains the domain that binds to CD40. Thus, soluble CD40-L can inhibit the interaction between CD40L and the CD40-bearing cell. This invention contemplates that sCD40-L may constitute the entire extracellular region of CD40-L, or a fragment or derivative containing the domain that binds to CD40.

The meaning of “chimeric”, “primatized” and “humanized” antibody and methods of producing them are well known to those of skill in the art. See, for example, PCT International Publication No. WO 90/07861, published Jul. 26, 1990 (Queen, et al.); and Queen, et al. Proc. Nat'l Acad. Sci.-USA (1989) 86: 10029). Methods of making primatized antibodies are disclosed, for example, in PCT International publication No. WO/02108, corresponding to International Application No. PCT/US92/06194 (Idec Pharmaceuticals); and in Newman, et al., Biotechnology (1992) 10:1455-1460, which are hereby incorporated by reference into this application.

Generally, a humanized antibody is an antibody comprising one or more complementarity determining regions (CDRs) of a non-human antibody functionally joined to human framework region segments. Additional residues associated with the non-human antibody can optionally be present. Typically, at least one heavy chain or one light chain comprises non-human CDRs. Typically, the non-human CDRs are mouse CDRs. Generally, a primatized antibody is an antibody comprising one or more complementarity determining regions (CDRs) of an antibody of a species other than a non-human primate, functionally joined to framework region segments of a non-human primate. Additional residues associated with the species from which the CDR is derived can optionally be present. Typically, at least one heavy chain or one light chain comprises CDRs of the species which is not a nonhuman primate. Typically, the CDRs are human CDRs. Generally, a chimeric antibody is an antibody whose light and/or heavy chains contain regions from different species. For example one or more variable (V) region segments of one species may be joined to one or more constant (C) region segments of another species. Typically, a chimeric antibody contains variable region segments of a mouse joined to human constant region segments, although other mammalian species may be used.

In another embodiment of this invention, the protein is soluble CD40 protein (sCD40), comprising the extracellular region of CD40, or portion thereof, or variant thereof. sCD40 inhibits the interaction between CD40L and CD40-bearing cells. sCD40 may be in monomeric or oligomeric form.

Variants can differ from naturally occurring CD40 or CD40 ligand in amino acid sequence or in ways that do not involve sequence, or both. Variants in amino acid sequence are produced when one or more amino acids in naturally occurring CD40 or CD40 ligand is substituted with a different natural amino acid, an amino acid derivative or non-native amino acid. Particularly preferred variants include naturally occurring CD40 or CD40 ligand, or biologically active fragments of naturally occurring CD40 or CD40 ligand, whose sequences differ from the wild type sequence by one or more conservative amino acid substitutions, which typically have minimal influence on the secondary structure and hydrophobic nature of the protein or peptide. Variants may also have sequences which differ by one or more non-conservative amino acid substitutions, deletions or insertions which do not abolish the CD40 or CD40 ligand biological activity. Conservative substitutions (substituents) typically include the substitution of one amino acid for another with similar characteristics such as substitutions within the following groups: valine, glycine; glycine, alanine; valine, isoleucine; aspartic acid, glutamic acid; asparagine, glutamine, serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively, charged (basic) amino acids include arginine, lysine and histidine. The negatively charged (acidic) amino acids include aspartic acid and glutamic acid.

Other conservative substitutions can be taken from Table 4, and yet others are described by Dayhoff in the Atlas of Protein Sequence and Structure (1988).

TABLE 4
Conservative Amino Acid Replacements

| For Amino Acid | Code | Replace with any of |
|---|---|---|
| Alanine | A | D-Ala, Gly, beta-ALa, L-Cys, D- |
| | | Cys |
| Arginine | R | D-Arg, Lys, homo-Arg, D-homo- |
| | | Arg, Met, D-M t, Ile, D-Ile, |
| | | Orn, D-Orn |
| Asparagine | N | D-Asn, Asp, D-Asp, Glu, D-Glu, |
| | | Gln, D-Gln |
| Aspartic Acid | D | D-Asp, D-Asn, Asn, Glu, D-Glu, |
| | | Gln, D-Gln |
| Cysteine | C | D-Cys, S-Me-Cys, Met, D-Met, Thr, |
| | | D-Thr |
| Glutamine | Q | D-Gln, Asn, D-Asn, Glu, D-Glu, Asp, |
| | | D-Asp |
| Glutamic Acid | E | D-Glu, D-Asp, Asp, Asn, D-Asn, |
| | | Gln, D-Gln |
| Glycine | G | Ala, D-Ala, Pro, D-Pro, Beta- |
| | | Ala, Acp |
| Isoleucine | I | D-Ile, Val, D-Val, Leu, D-Leu, |
| | | Met, D-Met |
| Leucine | L | D-Leu, Val, D-Val, Met, D-Met |
| Lysine | K | D-Lys, Arg, D-Arg, homo-Arg, D- |
| | | homo-Arg, Met, D-Met, Ile, D- |
| | | Ile, Orn, D-Orn |
| Methionine | M | D-Met, S-Me-Cys, Ile, D-Ile, |
| | | Leu, D-Leu, Val, D-Val, Norleu |
| Phenylalanine | F | D-Phe, Tyr, D-Thr, L-Dopa, His, D- |
| | | His, Trp, D-Trp, Trans 3,4 or |
| | | 5-phenylproline, cis 3,4 or 5 |
| | | phenylproline |
| Proline | P | D-Pro, L-I-thioazolidine-4- |
| | | carboxylic acid, D- or L-1- |
| | | oxazolidine-4-carboxylic acid |
| Serine | S | D-Ser, Thr, D-Thr, allo-Thr, |
| | | Met, D-Met, Met(O), D-Met(O), |
| | | Val, D-Val |
| Threonine | T | D-Thr, Ser, D-Ser, allo-Thr, |
| | | Met, D-Met, Met(O) D-Met(O), |
| | | Val, D-Val |
| Tyrosine | Y | D-Tyr, Phe, D-Phe, L-Dopa, |
| | | His, D-His |
| Valine | V | D-Val, Leu, D-Leu, Ile, D-Ile, |
| | | Met, D-Met |

Other variants within the invention are those with modifications which increase peptide stability. Such variants may contain, for example, one or more non-peptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D- instead of L-amino acids into the polypeptide may increase its resistance to proteases. See, e.g., U.S. Pat. No. 5,219,990.

The peptides of this invention may also be modified by various changes such as insertions, deletions and substitutions, either conservative or nonconservative where such changes might provide for certain advantages in their use.

In other embodiments, variants with amino acid substitutions which are less conservative may also result in desired derivatives, e.g., by causing changes in charge, conformation and other biological properties. Such substitutions would include for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge. When the result of a given substitution cannot be predicted with certainty, the derivatives may be readily, assayed according to the methods disclosed herein to determine the presence or absence of the desired characteristics.

Variants within the scope of the invention include proteins and peptides with amino acid sequences having at least eighty percent homology with the extracellular region of CD40 or the extracellular region of CD40 ligand. More preferably the sequence homology is at least ninety percent, or at least ninety-five percent.

Just as it is possible to replace substituents of the scaffold, it is also possible to substitute functional groups which decorate the scaffold with groups characterized by similar features. These substitutions will initially be conservative, i.e., the replacement group will have approximately the same size, shape, hydrophobicity and charge as the original group. Non-sequence modifications may include, for example, in vivo or in vitro chemical derivatization of portions of naturally occurring CD40 or CD40 ligand, as well as changes in acetylation, methylation, phosphorylation, carboxylation or glycosylation.

In a further embodiment the protein, including the extracellular region of CD40 ligand and CD40, is modified by chemical modifications in which activity is preserved. For example, the proteins may be amidated, sulfated, singly or multiply halogenated, alkylated, carboxylated, or phosphorylated. The protein may also be singly or multiply acylated, such as with an acetyl group, with a farnesyl moiety, or with a fatty acid, which may be saturated, monounsaturated or polyunsaturated. The fatty acid may also be singly or multiply fluorinated. The invention also includes methionine analogs of the protein, for example the methionine sulfone and methionine sulfoxide analogs. The invention also includes salts of the proteins, such as ammonium salts, including alkyl or aryl ammonium salts, sulfate, hydrogen sulfate, phosphate, hydrogen phosphate, dihydrogen phosphate, thiosulfate, carbonate, bicarbonate, benzoate, sulfonate, thiosulfonate, mesylate, ethyl sulfonate and benzensulfonate salts.

The soluble, monomeric CD40-L protein can comprise all or part of the extracellular region of CD40-L. The extracellular region of CD40-L contains the domain that binds to CD40. Thus, soluble CD40-L can inhibit the interaction between CD40L and the CD40-bearing cell. This invention contemplates that sCD40-L may constitute the entire extracellular region of CD40-L, or a fragment or derivative containing the domain that binds to CD40.

In another embodiment of this invention the protein comprising soluble extracellular region of CD40 or portion thereof further comprises an Fc region fused to the extracellular region of CD40 or portion thereof. In a specific embodiment the Fc region is capable of binding to protein A or protein G. In another embodiment the Fc region comprises IgG, IgG₁, IgG₂, IgG₃, IgG₄, IgA, IgA₁, IgA₂, IgM, IgD, or IgE.

In another embodiment of this invention, the sCD40 comprises CD40/Fc fusion protein. The fusion protein can be prepared using conventional techniques of enzymes cutting and ligation of fragments from desired sequences. Suitable Fc regions for the fusion protein are Fc regions that can bind to protein A or protein G, or that are capable of recognition by an antibody that can be used in purification or detection of a fusion protein comprising the Fc region. For example, the Fc region may include the Fc region of human IgG₁ or murine IgG₁. This invention also provides a nucleic acid molecule which encodes the CD40/Fc fusion protein.

The method of creating soluble forms of membrane molecules by recombinant means, in which sequences encoding the transmembrane and cytoplasmic domains are deleted, is well known. See generally Hammonds et al., U.S. Pat. No. 5,057,417. In addition, methods of preparing sCD40 and CD40/Fc fusion protein are well-known. See, e.g., PCT International Publication No. WO 93/08207:; Fanslow et al., “Soluble Forms of CD40 Inhibit Biologic Responses of Human B Cells, “J. Immunol., vol. 149, pp.655-60 (July 1992).

In an embodiment of this invention, the agent is a small molecule. As used herein a small molecule is a compound having a molecular weight between 20 Da and 1×10⁶ Da, preferably from 50 Da to 2 kDa.

In an embodiment of this invention, the agent is selected by a screening method.

In a specific embodiment the small molecule or other agent is selected by a screening method which comprises, isolating a cell sample, for example a sample of a biological fluid (e.g., blood) from an animal; culturing the sample under conditions permitting activation of CD40-bearing cells contained therein; contacting the sample with an amount of cells expressing a protein which is specifically recognized by monoclonal antibody 5c8 produced by the hybridoma having ATCC Accession no. HB 10916, or with a protein which is specifically recognized by monoclonal antibody 5c8 produced by the hybridoma having ATCC Accession no. HB 10916, effective to activate the CD40-bearing cells; contacting the sample with an amount of a small molecule (or other pharmaceutical compound or agent) effective to inhibit activation of the CD40-bearing cells if the small molecule is capable of inhibiting activation of the CD40-bearing cells; and determining whether the cells expressing the protein which is specifically recognized by monoclonal antibody 5c8 produced by the hybridoma having ATCC Accession no. HB 10916, or with the protein which is specifically recognized by monoclonal antibody 5c8 produced by the hybridoma having ATCC Accession no. HB 10916 activate the CD40-bearing cells in the presence of the small molecule (or other pharmaceutical compound or agent). The cell sample may be isolated from diverse tissues, including cell lines in culture or cells isolated from an animal, such as dispersed cells from a solid tissue, cells derived from a bone marrow biopsy, or cells isolated from a body fluid such as blood or lymphatic fluid.

In another specific embodiment the agent (molecule) is selected based on a, three-dimensional structure of soluble extracellular region of CD40 ligand or portion thereof capable of inhibiting interaction between CD40 ligand and CD40 on the cells. The agent may be selected from a library of known agents, modified from a known agent based on the three-dimensional structure, or designed and synthesized de novo based on the three-dimensional structure. In specific embodiments the agent (molecule) is designed by structure optimization of a lead inhibitory agent based on a three-dimensional structure of a complex of the soluble, extracellular region of CD40 ligand or portion thereof with the lead inhibitory agent. A lead inhibitory agent is a molecule which has been identified which, when it is contacted with CD40 ligand or portion thereof, binds to and complexes with the soluble extracellular region of CD40 ligand, CD40, or portion thereof, thereby decreasing the ability of the complexed or bound CD40 ligand or CD40 ligand portion to activate CD40-bearing cells. In another embodiment, a lead inhibitory agent may act by interacting with either the extracellular region of CD40 ligand, CD40, or in a tertiary complex with both a portion of CD40 ligand and CD40, decreasing the ability of the complexed CD40, ligand-CD40 to activate the CD40-bearing c lls. In the methods of th invention, the CD40 ligand may be either soluble or bound to cells such as activated T cells, and may be either full length native CD40 ligand or portions thereof. Decreased ability to activate CD40-bearing cells may be measured in different ways one way it may be measured is by showing that CD40 ligand, in the presence of inhibitor, causes a lesser degree of activation of CD40-bearing cells, as compared to treatment of the cells with a similar amount of CD40 ligand without inhibitor under similar conditions. Decreased ability to activate CD40-bearing cells may also be indicated by a higher concentration of inhibitor-CD40 ligand complex being required to produce a similar degree of activation of CD40-bearing cells under similar conditions, as compared to unbound CD40 ligand. At the extreme, the inhibitor-contacted CD40 ligand may be unable to activate CD40-bearing cells at concentrations and under conditions which allow activation of these cells by unbound CD40 ligand or a given portion thereof.

The agent (small molecule) can be selected by a computational screening method using the crystal structure of a soluble fragment of the extracellular domain of human CD40L containing residues Gly116-Leu261 of SEQ ID NO:1.

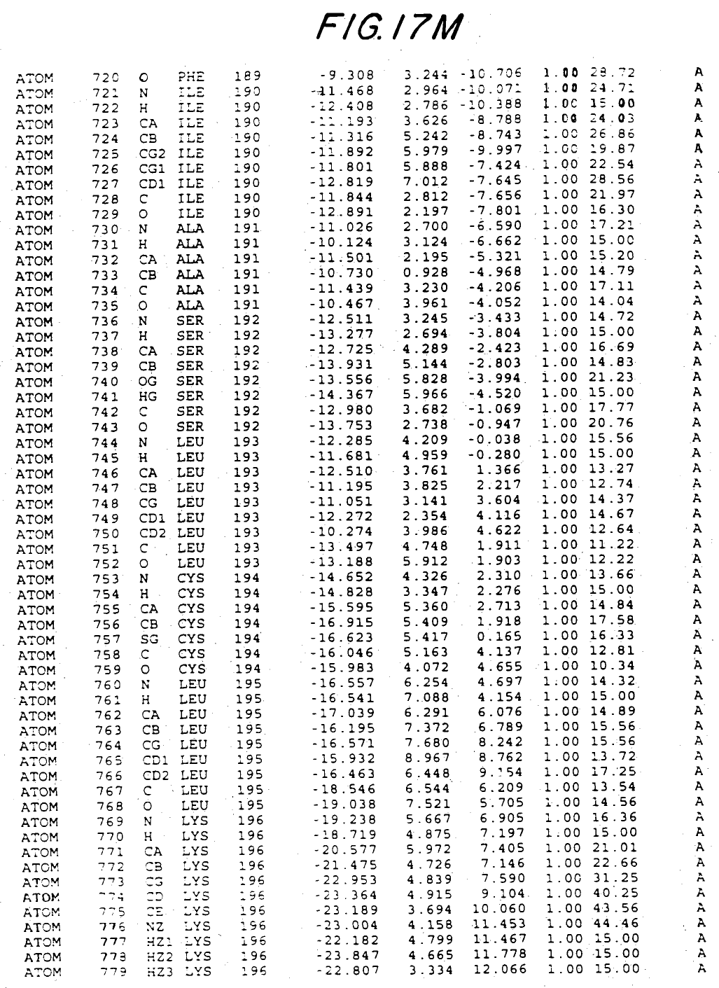

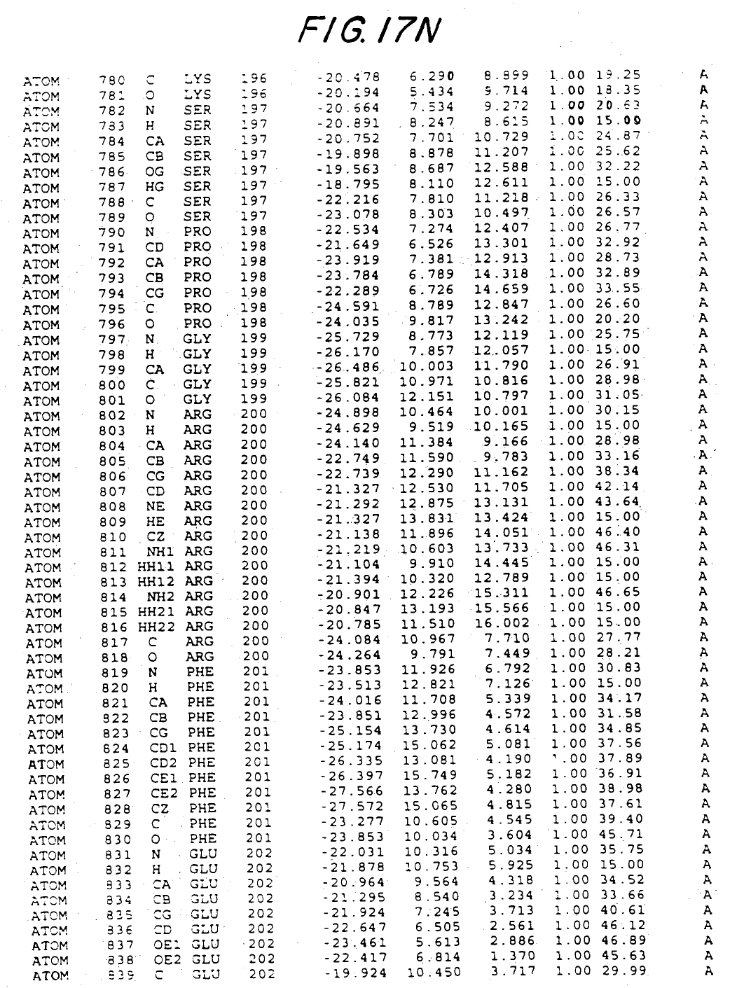

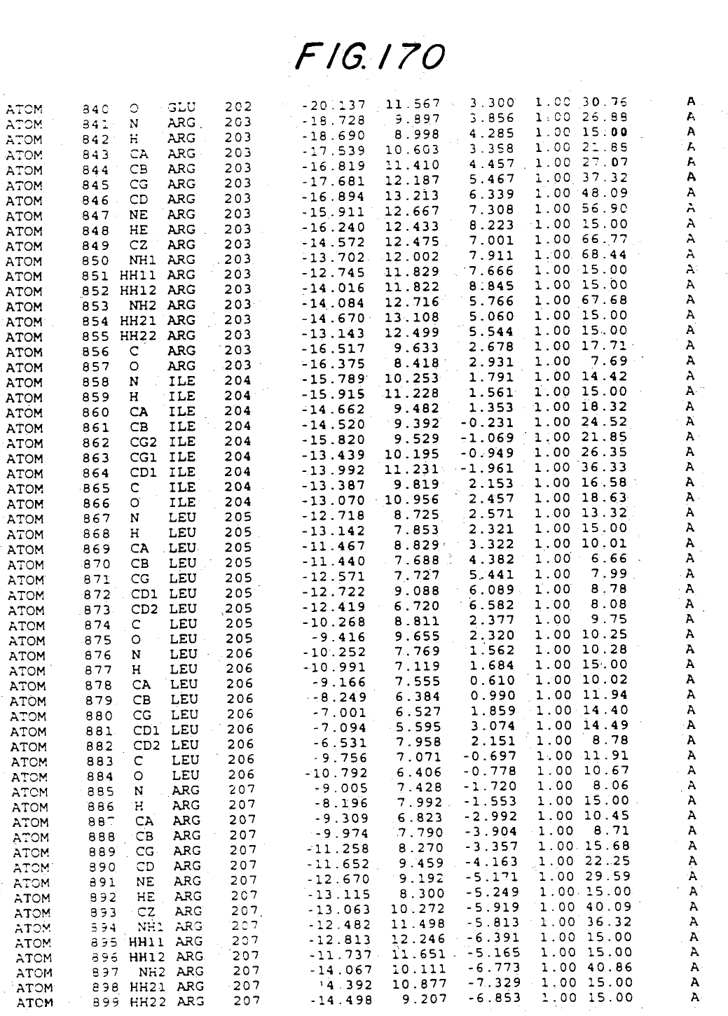

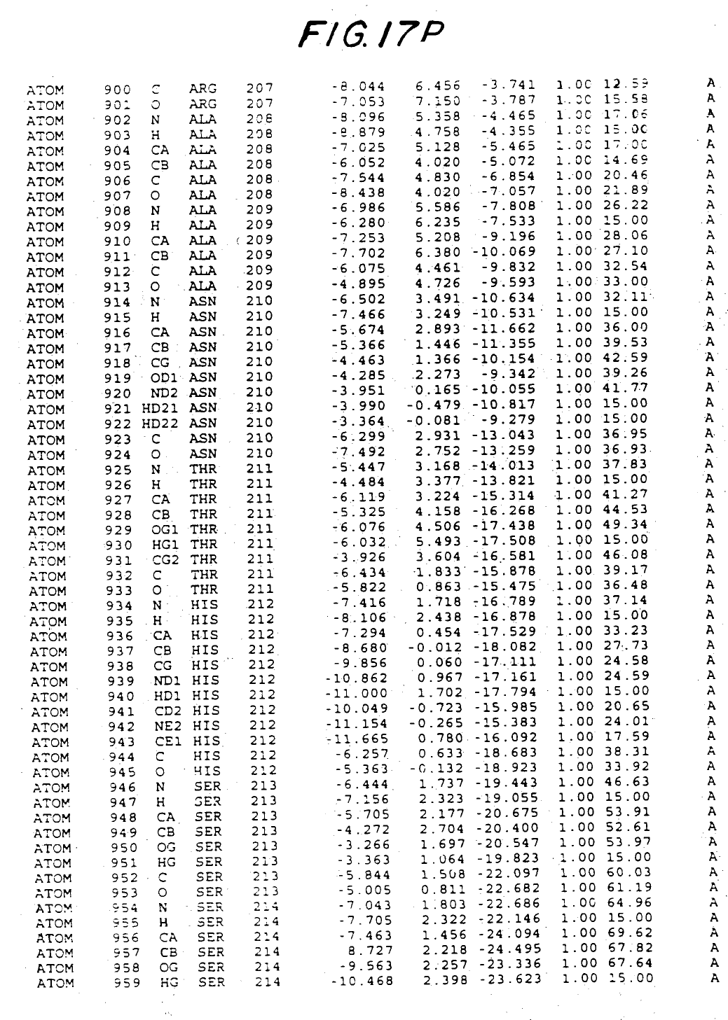

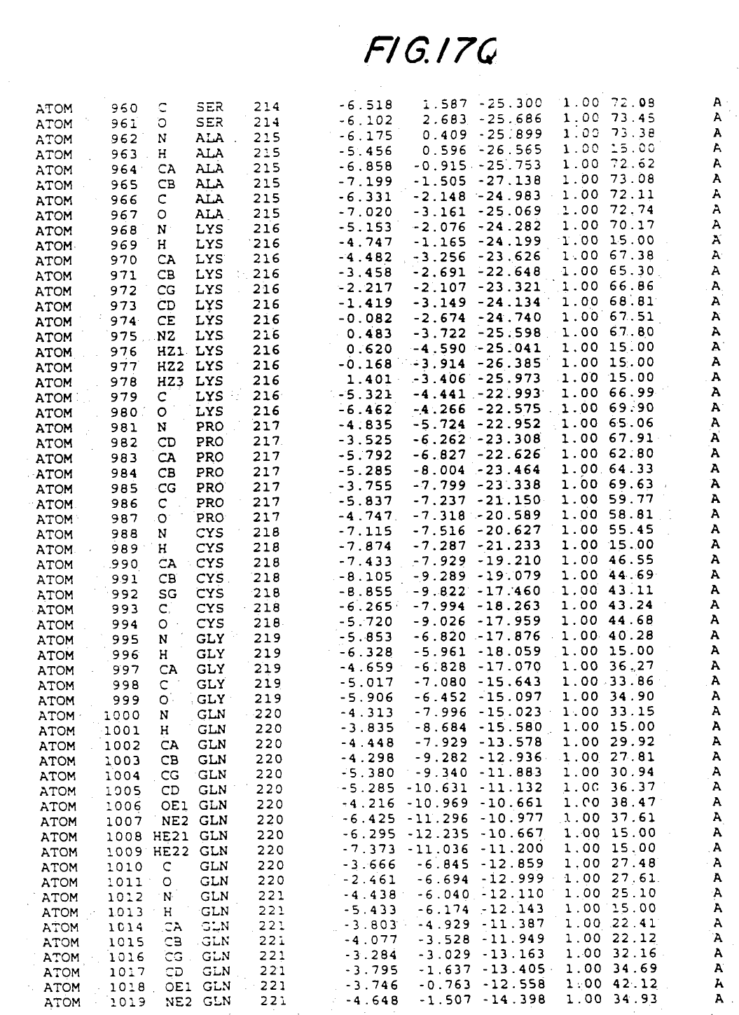

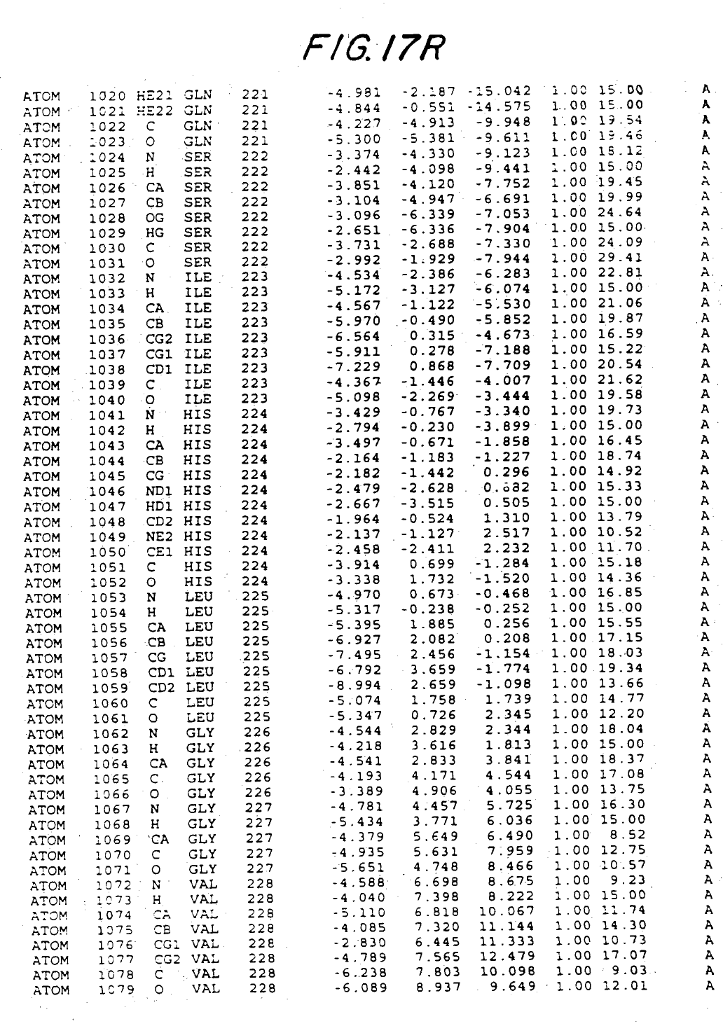

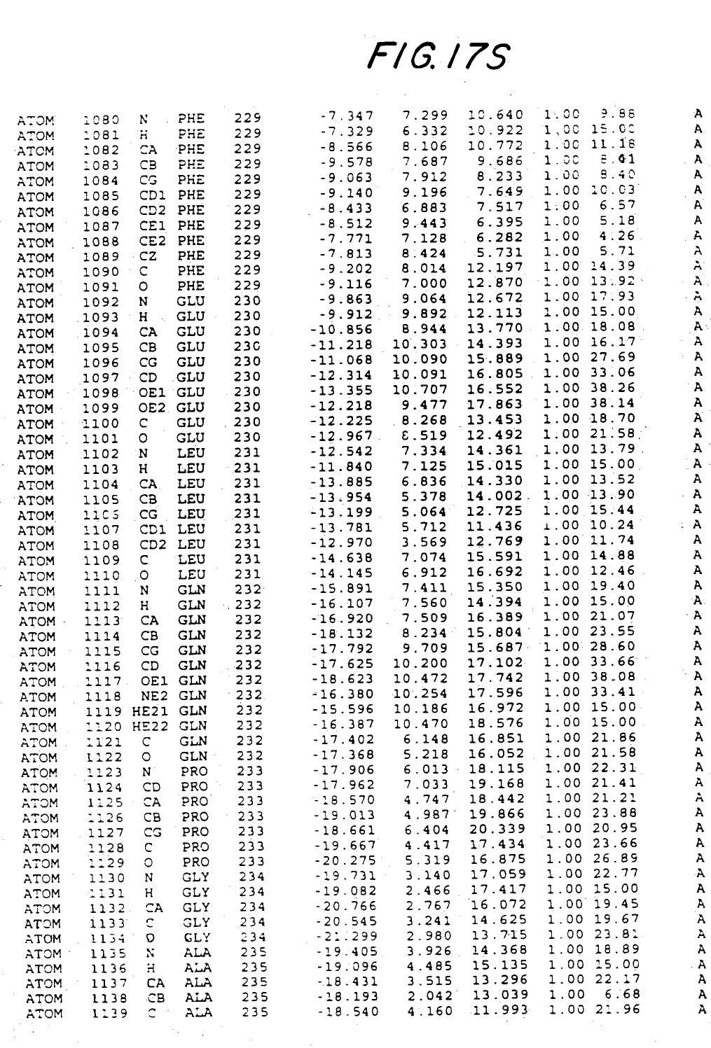

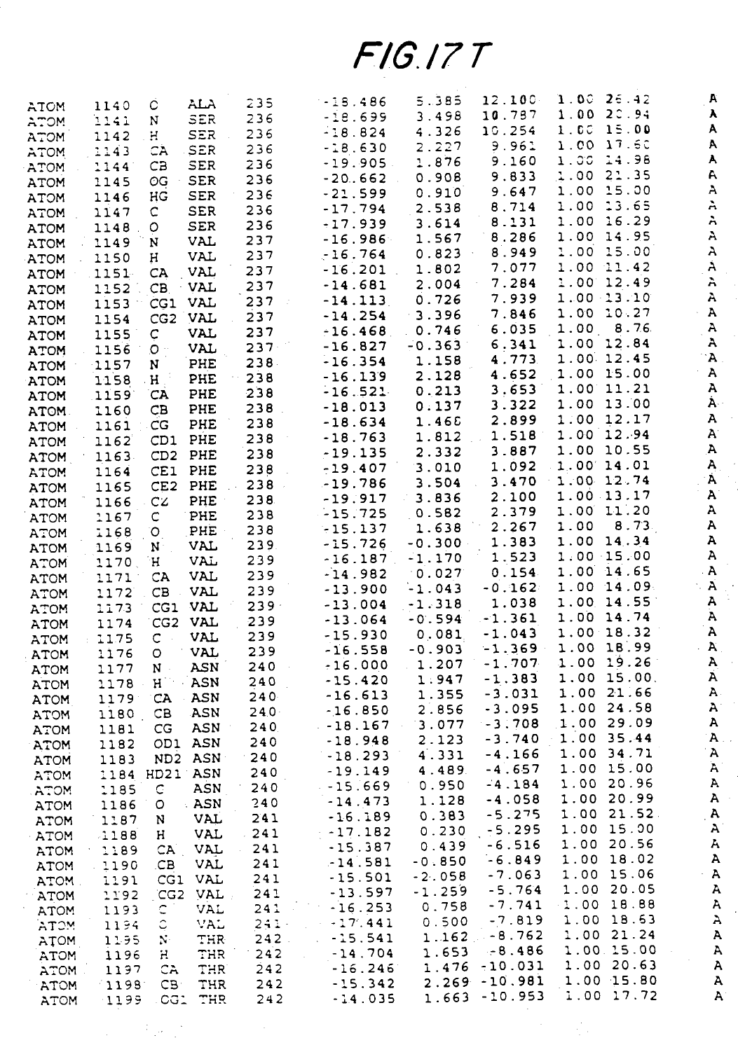

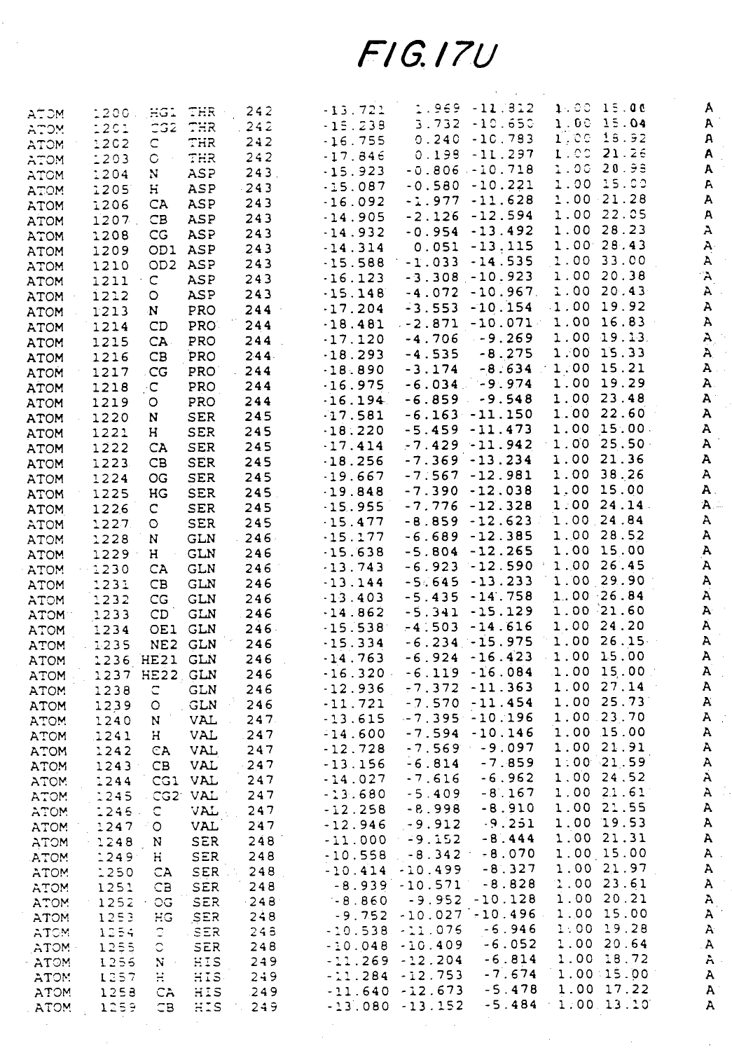

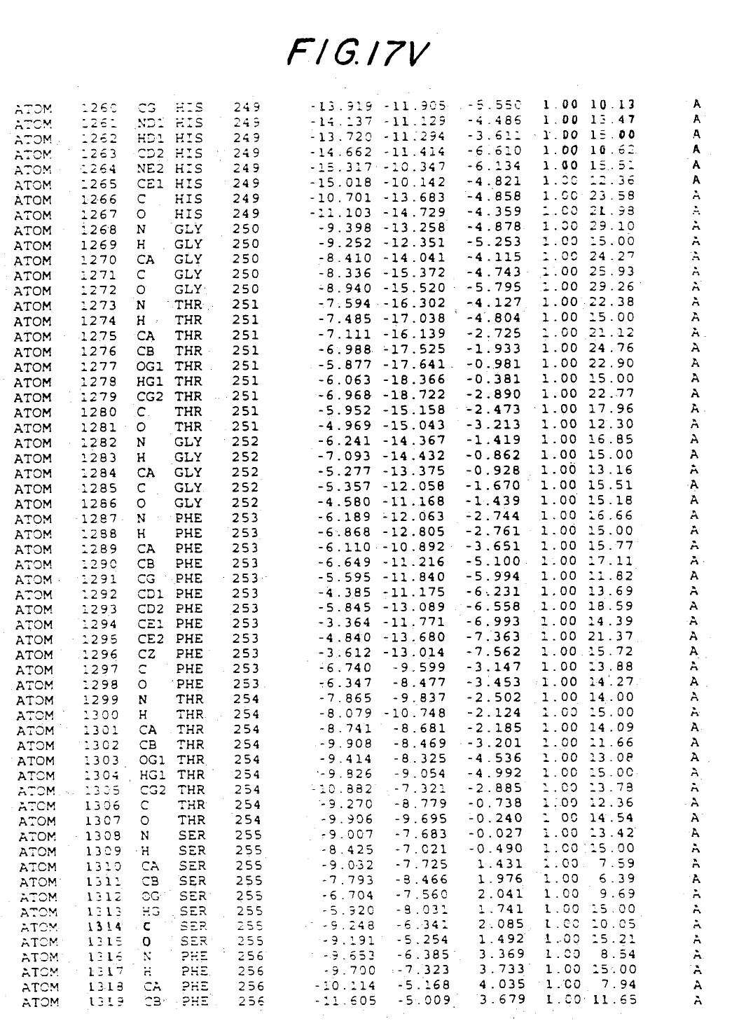

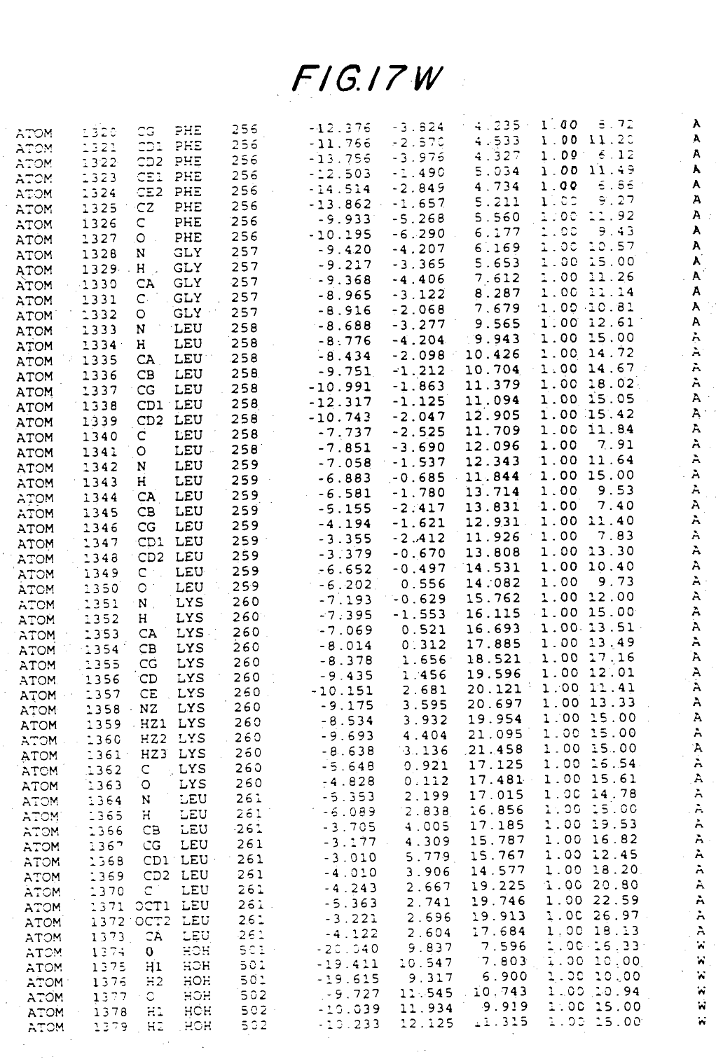

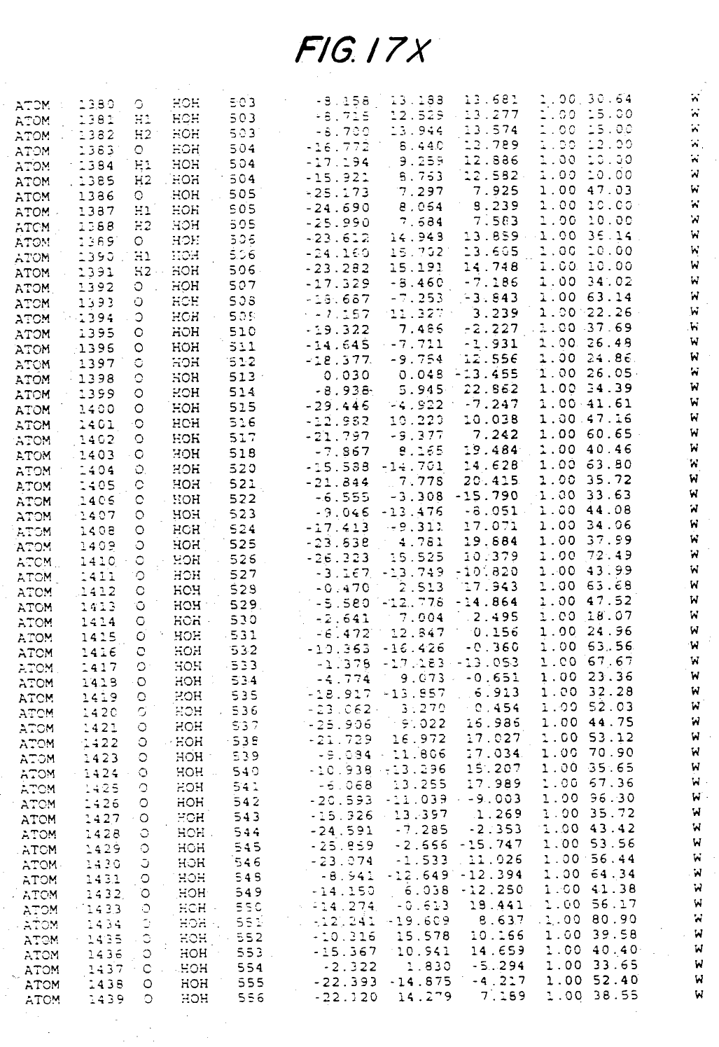

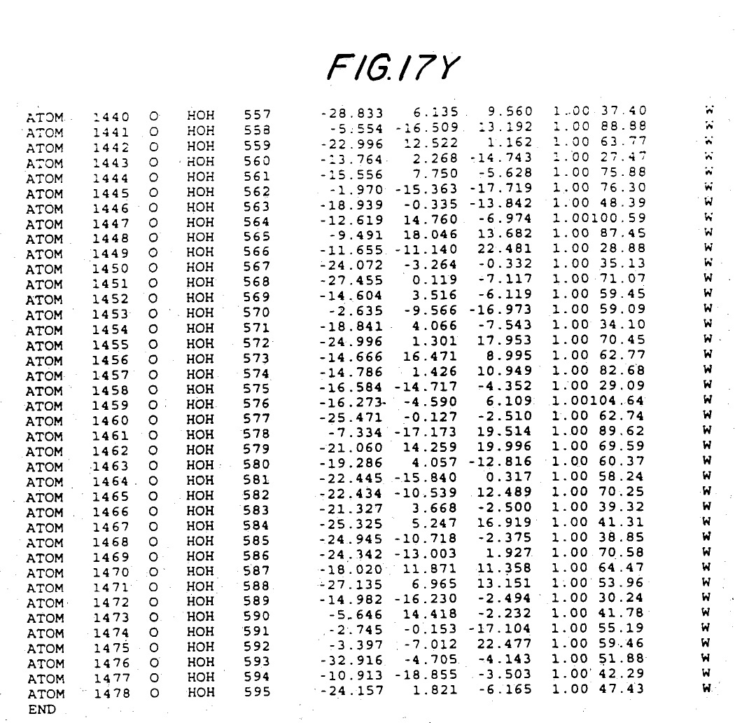

The crystal structure to be used with the screening method can be determined at 2 Å resolution by the method of molecular replacement. In brief, a soluble fragment of the extracellular domain of human CD40 ligand containing amino acid residues Gly 116 to the C-terminal residue Leu 261 are first produced in soluble form, then purified and crystallized. The crystals can be tested for diffraction capacity on the X-ray beam of an Elliot GX-13 generator. Molecular replacement and refinement can be done with the XPLOR program package and QUANTA (Molecular Simulations, Inc.) Software. In particular, a 3-dimensional model of human sCD40L can be constructed using the murine CD40L model using QUANTA protein homology modeling software. This model can then be used as a probe for molecular replacement calculations and refined using XPLOR. This method of determining the crystal structure of sCD40L is described in more detail in Karpusas. et al., “2 Å crystal structure of an extracellular fragment of human CD40 ligand,” Structure (October 1995) 3(10):1031-1039. The atomic coordinates of sCD40L(116-261) are provided in FIGS. 17A-Y. The screening method for selecting an agent includes computational drug design and iterative structure optimization, as described below.

The agent may be a small molecule inhibitor selected using computational drug design. Using this method, the sCD40L crystal structure coordinates are used as an input for a computer program, such as DOCK, which outputs a list of small molecule structures that are expected to bind to CD40L. Use of such computer programs are well-known. See, e.g.,. Kuntz, “Structure-Based Strategies for drug design and discovery,” Science, vol. 257, p. 1078 (1992). The list of small molecule structures can then be screened by biochemical assays for CD40L binding. Competition-type biochemical assays, which are well known, can be used. See, e.g., Bajorath et al., “Identification of residues of CD40 and its ligand which are critical for the receptor-ligand interaction,” Biochemistry, 34, p. 1833 (1995). The structures that are found to bind to CD40L can thus be used as agents for the present invention. The agent may also be a modified small molecule, determined by interactive cycles of structure optimization. Using this approach, a small molecule inhibitor of CD40L found using the above computational approach or other approach can be co-crystallized with sCD40L and the crystal structure of the complex solved by molecular replacement. The information revealed through molecular replacement can be used to optimize the structure of the small molecule inhibitors by clarifying how the molecules interact with CD40L. The small molecule may be modified to improve its physiochemical properties, including specificity and affinity for CD40L.

In an embodiment of this invention the agent specifically binds to CD40 on the cell surface. In a specific embodiment the agent is a protein, for example an antibody or the extracellular region of CD40 ligand. The antibody may be a polyclonal or monoclonal antibody. It is preferred that the monoclonal antibody be chimeric or humanized. It may also be primatized.

In Vivo Use

This invention provides a method of inhibiting activation by CD40 ligand of cells bearing CD40 on the cell surface, in a subject, comprising administering to the subject an agent capable of inhibiting interaction between CD40 ligand and the cells, in an amount effective to inhibit activation of the cells in the subject. In one embodiment, the cells bearing CD40 on the cell surface are cells other than B cells. In another embodiment, they are plasma cells, including differentiated plasma cells such as myeloma cells.

In specific embodiments of this invention, the non-B cell, CD40-bearing cells are fibroblasts, endothelial cells, epithelial cells, T cells, basophils, macrophages, Reed-Steinberg cells, or dendritic cells. In a more specific embodiment the epithelial cells are keratinocytes. In another embodiment, the macrophages are foam cells (lipid-laden macrophages). Foam cells play a role in autoimmune diseases, for example rheumatoid arthritis and atherosclerosis.

In an embodiment of this method, the agent is a protein.

In a more specific embodiment, the protein comprises an antibody or portion thereof, for example a Fab, F(ab′)₂, complementarity determining region (CDR) light and/or heavy chain, antibody variable region light and/or heavy chain, or a portion thereof capable of specifically binding to CD40 ligand or CD40 ligand cell-surface receptor, or to CD40.One example of an anti-CD40 antibody is the monoclonal mouse anti-human CD40, available from Genzyme Customer Service (Product 80-3702-01, Cambridge, Mass.). The antibody can be a monoclonal or polyclonal antibody. In embodiments of this invention, the monoclonal antibody is a chimeric antibody, a humanized antibody, or a primatized antibody. In another embodiment the portion of the antibody comprises a single chain antibody. A single chain antibody is made up of variable regions linked by protein spacers in a single protein chain.

In an embodiment of the above-described method, the agent specifically binds to the antigen to which monoclonal antibody 5c8 (ATCC Accession No. HB 10916) specifically binds. In a specific embodiment, the agent is monoclonal antibody 5c8 (ATCC Accession No. HB 10916).

The compounds of this invention may be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, topical, or inhaled. Sustained release administration is also specifically included in the invention, by such means as depot injections of erodible implants directly applied during surgery.

The compounds are administered at any dose per body weight and any dosage frequency which is medically acceptable. For example, acceptable dosage for the compound of this invention (especially for the antibody or antibody portion of this invention) includes a range of between about 0.01 and 200 mg/kg subject body weight. A dosage range is between about 0.1 and 50 mg/kg. In a still more specific embodiment the dose is between about 1 and 30 mg/kg. The dosage is repeated at intervals ranging from each day to every other month. One dosing regimen is to administer a compound of the invention daily for the first three days of treatment, after which the compound is administered every 3 weeks, with each administration being intravenously at 5 or 10 mg/kg body weight.

Another regime is to administer a compound of the invention daily intravenously at 5 mg/kg body weight for the first three days of treatment, after which the compound is administered subcutaneously or intramuscularly every week at 10 mg per subject. Another regime is to administer a single dose of the compound of the invention parenterally at 20 mg/kg body weight, followed by administration of the compound subcutaneously or intramuscularly every week at 10 mg per subject.

The compounds of the invention may be administered as a single dosage for certain indications such as preventing immune response to an antigen to which a subject is exposed for a brief time, such as an exogenous antigen administered on a single day of treatment. Examples of such an antigen would include coadministration of a compound of the invention along with a gene therapy vector, or a therapeutic agent such as an antigenic pharmaceutical or a blood product. In indications where antigen is chronically present, such as in controlling immune reaction to transplanted tissue or to chronically administered antigenic pharmaceuticals, the compounds of the invention are administered at intervals for as long a time as medically indicated, ranging from days or weeks to the life of the subject.

This invention provides a method of inhibiting an inflammatory response in a subject, comprising the above-described method of inhibiting activation by CD40 ligand of cells, other than B cells, bearing CD40 on the cell surface (e.g., fibroblast cells, endothelial cells, or keratinocyte cells) in a subject. Inflammatory responses are characterized by redness, swelling, heat and pain, as consequences of capillary dilation with edema and migration of phagocytic leukocytes. Inflammation is further defined by Gallin (Chapter 26, Fundamental Immunology, 2d ed., Raven Press, New York, 1989, pp. 721-733), which is hereby incorporated by reference.

This method is effective in inhibiting activation of any fibroblasts. In particular embodiments, the fibroblasts are synovial membrane fibroblasts, dermal fibroblasts, pulmonary fibroblasts, or liver fibroblasts. In particular embodiments, the condition dependent on CD40 ligand-induced activation of fibroblast cells is selected from the group consisting of arthritis, scleroderma, and fibrosis (e.g. fibrotic diseases of the liver and lung). In an embodiment of this invention, the fibrotic disease of the lung is caused by rheumatoid arthritis or scleroderma.

In an embodiment of this invention the arthritis is rheumatoid arthritis, non-rheumatoid inflammatory arthritis, arthritis associated with Lyme disease, or osteoarthritis. In another specific embodiment, the fibrosis is pulmonary fibrosis, hypersensitivity pulmonary fibrosis, or pneumoconiosis. In another specific embodiment, the fibrotic disease of the liver is Hepatitis-C, Hepatitis-B, Hepatitis non-B non-C, cirrhosis, or cirrhosis of the liver secondary to a toxic insult, drugs, a viral infection, or an autoimmune disease. Alcohol consumption is one example of toxic insult which can cause cirrhosis of the liver. One example of a drug that can cause cirrhosis of the liver is Bleomycin. Others are known in the art.

Examples of viral infections which can cause fibrotic disease of the liver include, among others known to the art, Hepatitis B, Hepatitis C, and Hepatitis non-B non-C. Examples of autoimmune diseases which can cause fibrotic disease of the liver include, among others known to the art, primary biliary cirrhosis, and Lupoid hepatitis (autoimmune hepatitis). In specific embodiments the pulmonary fibrosis is pulmonary fibrosis secondary to adult respiratory distress syndrome (ARDS), drug-induced pulmonary fibrosis, idiopathic pulmonary fibrosis, or hypersensitivity pneumonitis; the pneumoconiosis is asbestosis, siliconsis, or Farmer's lung as well as other pneumoconioses that are known in the art to which this invention pertains.

This invention provides a method of treating a condition dependent an CD40 ligand-induced activation of endothelial cells in a subject, comprising the above-described method of inhibiting activation of endothelial cells by CD40 ligand in a subject.

In embodiments of this invention the condition dependent on CD40 ligand-induced activation of endothelial cells is selected from the group consisting of atherosclerosis, reperfusion injury, allograft rejection, organ rejection, and chronic inflammatory autoimmune diseases.

In a specific embodiment the atherosclerosis is accelerated atherosclerosis associated with organ transplantation. In situ CD40 and CD40L expression in accelerated atherosclerosis associated with transplant rejection have been studied. Frozen sections of coronary arteries from 4 heart transplant patients that required retransplantation due to accelerated atherosclerosis were analyzed by routine immunohistochemistry utilizing anti-CD40 mAb G28.5, anti-CD40L mAb 5C8 or control mAbs. Routine H & E staining revealed the typical intimal hyperplasia, smooth muscle cell proliferation, and inflammatory cell infiltration associated with the disease. CD40 was widely expressed in the lesions: endothelial cells, foam cells and infiltrating inflammatory cells all express CD40. CD40L immunoreactivity was observed as discrete, faint staining of infiltrating mononuclear cells, presumably CD4+ T cells. Together, these studies demonstrate the presence of CD40L+ mononuclear cells and CD40+ endothelial cells, foam cells, and inflammatory cells in situ in lesions of accelerated atherosclerosis associated with transplantation.

In another specific embodiment the chronic inflammatory autoimmune disease is vasculitis, rheumatoid arthritis, scleroderma, or multiple sclerosis.

This invention provides a method of treating a condition dependent on CD40 ligand-induced activation of keratinocytes in a subject, comprising the above-described method of inhibiting activation of keratinocyte cells by CD40 ligand in a subject.

In a specific embodiment the condition dependent on CD40 ligand-induced activation of keratinocytes is psoriasis.

This invention provides a method of treating a condition dependent on CD40 ligand-induced activation of macrophages in a subject, comprising th above-described method of inhibiting activation of macrophages by CD40 ligand in a subject. In specific embodiments, the condition dependent on CD40 ligand-induced activation of macrophages is atherosclerosis or rheumatoid arthritis.

The subject which can be treated by the above-described methods is an animal. Preferably the animal is a mammal. Examples of mammals which may be treated include, but are not limited to, humans; rodents such as the murine animals rats and mice, as well as rabbits, and guinea pig; cow; horse; sheep; goat; pig; dog and cat.

This invention also provides a method of treating a condition dependent on CD40 ligand-induced activation of plasma cells in a subject (including malignant plasma cells), comprising administering to the subject an, agent capable of inhibiting interaction between CD40 ligand and the cells, in an amount effective to inhibit activation of the cells in the subject. Plasma cells are differentiated B cells. In a specific embodiment the condition is multiple myeloma.

This invention provides a method of promoting the growth of cells bearing CD40 on the cell, comprising contacting the cells with an amount of CD40 ligand effective to promote growth of the cells. In an embodiment the cells are cells bearing CD40 on the cell surface other than B cells. In specific embodiments the non-B cells bearing CD40 on the cell surface are endothelial cells, fibroblasts, epithelial cells, T cells, or basophils. In another embodiment the cells are plasma cells, including differentiated plasma cells such as myeloma cells.

This invention further provides a pharmaceutical composition comprising a therapeutically effective amount of the agent described herein capable of inhibiting interaction between CD40 ligand and cells bearing CD40 on the cell surface, and a pharmaceutically acceptable carrier.

This invention will be better understood from the Experimental Details which follow. However, one skilled in the art will readily appreciate that the specific methods and results discussed are merely illustrative of the invention as described more fully in the claims which follow thereafter.

Experimental Details

First Series of Experiments

Materials and Methods

Patients Studied

All RA patients studied met the American College of Rheumatology criteria for RA (19). The diagnosis of OA was established by the patients' physicians utilizing clinical and radiographic criteria. One patient with chronic inflammatory arthritis (IA) of unknown etiology was also studied.

Monoclonal Antibodies and T Cell Lines

The: IgG2a murine anti-CD40L mAb (5C8) was previously generated (3). Hybridomas anti-MHC Class I (W6/32), anti-MHC Class II (L243), anti-CD14 (3C10), anti-CD40 (G28.5) and anti-CD45 (GAP 8.3) were purchased from American Type Culture Collection (ATCC) (Rockville, Md.). Hybridoma ascites was purified on a Protein G column (Pharmacia, Piscataway, N.J.). Anti-CD13 and anti-CD54 mAbs were purchased from Biosource International (Camarillo, Calif). Anti-CD106 mAb was kindly provided by Biogen (Cambridge, Mass) and biotinylated as previously described (20). Isotype control mAbs utilized for FACS analysis were purchased from Becton-Dickinson (San Jose, Calif.) or Caltag (South San Francisco, Calif.). P1.17 is a control IgG2a murine mAb obtained from Biogen and utilized for functional studies.

D1.1 is a Jurkat T cell subclone that constitutively expresses CD40L (3, 21). B2.7 is a CD40L⁻ Jurkat subclone (3, 21). CD40L⁺ Jurkat B2.7 transfectants-expressing full length CD40L protein were generated as previously reported (20).

Isolation of FibroBlasts

Synovial membrane was obtained from 6 RA or 8 OA patients undergoing joint replacement surgery. SM from one patient with IA was collected at arthroscopy. SM was cut into small pieces and cultured in 100 mm tissue culture petri dishes (Corning, Corning, N.Y.) or 25 cm² flasks (Costar, Cambridge, Mass.) with Isocove's Modified Dulbecco's Media (Gibco, Grand Island, N.Y.) supplemented with 10% FCS (Summit Biotechnology, Ft. Collins, Colo.) and 1% penicillin-streptomycin (Sigma, St. Louis, Mo.) (10% FM). Synoviocytes were allowed to adhere for several days at which time tissue debris and non-adherent cells were removed. Synoviocytes were grown to confluence and passaged by treatment with 1% trypsin-EDTA (Sigma). Synoviocytes were studied between 1-6 passages in vitro. A normal dermal fibroblast line frozen following the second passage (CCD 965SK) was purchased from ATCC. Dermal fibroblast lines were studied between 2-4 passages.

Studies on the Effects of Cytokines on Fibroblast CD40 Expression

To study the effects of cytokines on fibroblast CD40 expression, cells were cultured in 6 well plates (Nunc, Denmark) and grown to near confluence. The media was aspirated and fibroblasts then cultured with the indicated concentrations of rINF-γ (Biogen), rIL-1α (R & D, Minneapolis, Minn.), rTNF-α (Upstate Biotechnology, Lake Placid, N.Y.), rIL-4 (Biosource International), rGM-CSF (Immunex, Seattle, Wash.) or combinations of cytokines in 3 ml of 10% FM. At the indicated time points, the media was aspirated, the cells washed once with saline and 1 ml of 1% trypsin-EDTA added. to the wells. After 7 minutes cold 10% FM was added to the wells and the cells collected for FACS analysis.

Studies on Functional Consequences of fibroblast CD40 Ligation.

To determine the effect of CD40 ligation on the expression of fibroblast cell surface molecules, fibroblasts were cultured in 6 well plates as described above. When the fibroblasts were near confluence 1×10⁶ CD40L⁺ Jurkat D1.1 cells, CD40L Jurkat B2.7 cells or CD40L⁺ Jurkat B2.7 transfectants were added to the culture. Where indicated, D1.1 cells were pretreated with anti-CD40L mAb 5C8 (10 μg/ml) or isotype control mAb P1.17 (10. μg/ml) prior to the addition to fibroblasts. After 24 hours the cells were collected by trypsinization and two-color FACS analyses performed.

For studies determining the effect, of CD40 ligation on fibroblast proliferation, approximately 5×10³ cells were added to flat bottom 96 well plates (Nunc) in 10% FM. After 18 hours the media was changed to 1% FM and rINF-γ 1000 U/ml added to the indicated cells. After an additional 18 hours, 1×10⁵ mitomycin-C (Sigma) treated CD40L⁺ Jurkat B2.7 transfectants or CD40L Jurkat B2.7 cells in 1% FM were added to the fibroblasts. Anti-CD40L mAb 5C8 (5 μg/ml) or control mAb P1.17 (5 μg/ml) were also added to some wells as indicated. 10% FM was added to some cells as a control for the induction of SM fibroblast proliferation. Cultures were maintained for an additional 48 hours and pulsed with 1 μCi ³H thymidine for the last 18 hours of the experiment. Following trypsinization, ³H thymidine incorporation was determined by harvesting onto glass fiber filter strips (Cambridge Technologies, Watertown, Mass.) and scintillation counting (BetaCounter, Pharmacia).

To determine the is effect of CD40 ligation on IL-6 production, a bioassay utilizing the IL-6 responsive murine B cell line B9 was performed (22). Equal numbers of fibroblasts in 10% FM were seeded in 96 well plates as mentioned above. After adhering overnight, 1×10⁵ mitocycin-C treated CD40L⁺ Jurkat D1.1 cells, CD40L Jurkat B2.7 cells or CD40L⁺ Jurkat B2.7 transfectants were added to the fibroblasts. Where indicated, D1.1 cells were pretreated with anti-CD40L mAb 5C8 (10 μg/ml) or control mAb P1.17 (10 μg/ml). Control wells consisted of Jurkat cells cultured alone. After 48 hours, serial dilutions of fibroblast or control supernatants or rIL-6 were added to 7.5×10³ B9 cells in 96 well plates. B9 cells were maintained in culture for 96. hours, pulsed with 1 μCi ³H thymidine for the last 18 hours and harvested as mentioned above.

Cytofluorographic Analysis

The methods utilized for cytofluorographic analysis have been previously described (21). In all experiments the cells were first treated with aggregated human immunoglobulin (Enzyme International, Fallbrook, Calif.) to block non-specific Ig binding. For single-color FACS analysis, cells were stained with saturating concentrations of primary antibody for 30-60 minutes at 4° C. Following washing, FITC conjugated F(ab)₂ goat anti-mouse IgG (Cappel, Cochranville, Pa.) was added for 30-60 minutes at 4° C. The cells were washed and fixed with 1% formaldehyde prior to FACS analysis. For two-color FACS analysis, cells were simultaneously stained with the indicated FITC or PE conjugated mAbs for 30-60 minutes at 4° C. Fluorescence intensity was measured on a FACScan cytofluorograph with the Consort-30 software (Becton-Dickinson, Mountainview, Calif.). Mean fluorescence intensity (MFI) refers to values normalized to the log scale as calculated by Becton-Dickinson C30 software.

Results

Expression of CD40 n Cultured SM or Dermal Fibroblasts.

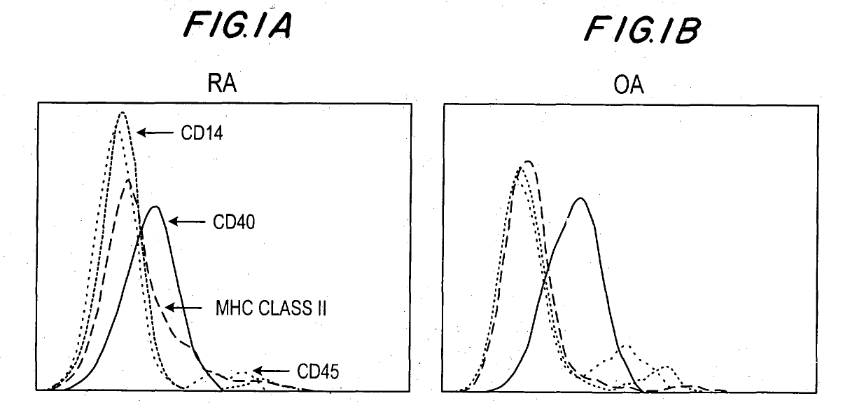

To determine whether SM fibroblasts express CD40, SM derived from 6 RA, 1 IA, or, 8 OA patients was first minced and placed in culture after which non-adherent cells were discarded. As expected, primary cultures of adherent cells were pleiomorphic with regard to morphology and phenotype. A minority of cells assumed a stellate morphology or a rounded appearance characteristic of macrophages. However, the majority of cells in primary culture had fibroblast-like morphology and phenotype, i.e., CD45⁻CD14⁻MHC Class II⁻ (FIG. 1). Virtually all cells had fibroblast-like morphology and phenotype following 2-3 passages in vitro.

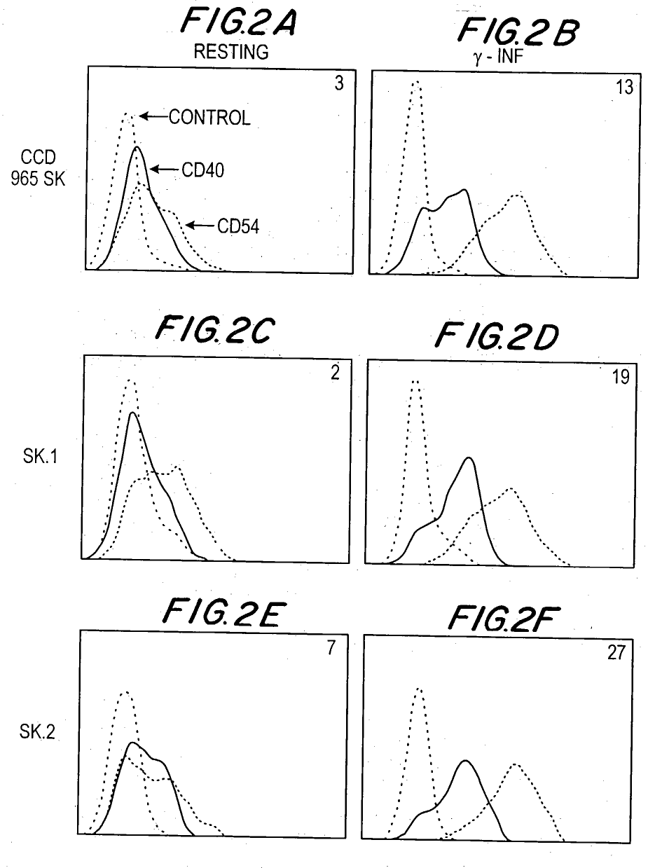

Five RA fibroblast lines were studied for CD40 expression following the first or second passage in vitro and were CD40⁺ by FACS analysis (FIG. 1). An IA fibroblast line similarly expresses CD40 (table 1). One RA fibroblast line had been in culture for 2 months prior to analysis and was CD40⁻ (data not shown). Eight OA fibroblast lines were studied for CD40 expression following the first or second passage in vitro and all were CD40⁺ (FIG. 1). To determine if fibroblast CD40, expression was restricted to SM fibroblasts, normal dermal fibroblasts were analyzed for CD40 expression following 2-4 passages in vitro. To variable degrees, all 3 dermal fibroblast lines studied also express cell surface CD40 molecules (FIG. 2). However, CD40 expression on synovial membrane or dermal fibroblasts decreased with increasing time in culture such that some fibroblast lines became CD40⁻ after 3-4 passages (data not shown). These studies demonstrate that dermal fibroblasts or SM fibroblasts isolated from patients with various arthritides can express CD40 in vitro.

Effect of Cytokines on Fibroblast CD40 Expression

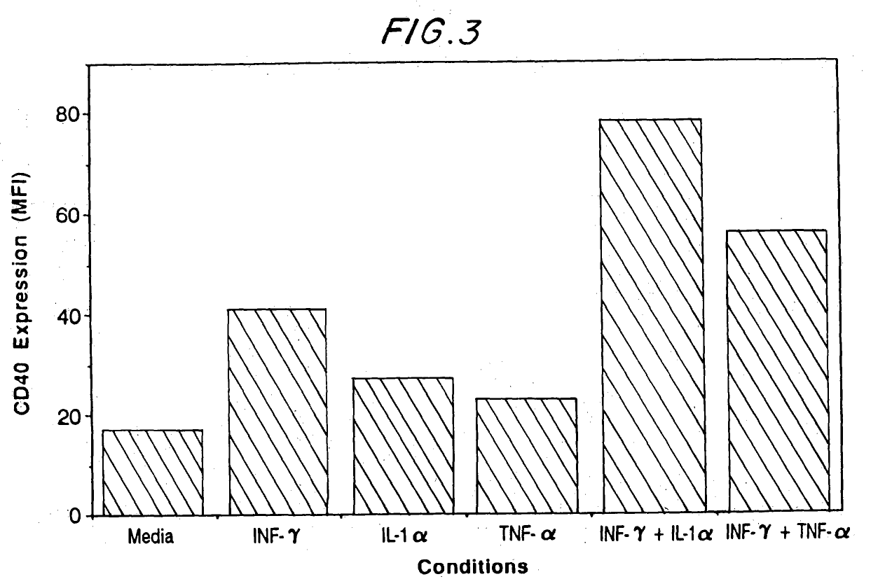

Interferon-γ (INF-γ) is known to upregulate CD40 expression on B cells (23), macrophages (12) and thymic epithelial cells (15). Moreover, IL-1α or TNF-α upregulates CD40 expression on thymic epithelial cells (15). Therefore, it was next asked if rINF-γ, rIL-1α or rTNF-α regulates CD40 expression, on cultured SM fibroblasts. Cells were cultured with the indicated cytokines and CD40 expression determined by FACS analysis. As a control for the effects of these cytokines on the expression of SM fibroblast cell surface molecules, CD54 (ICAM-1) expression was also determined (24). rINF-γ upregulates SM fibroblast CD40 expression (table 1 and FIG. 3). In contrast, rIL-1α and rTNF-α have minimal effect on SM fibroblast CD40 expression (table 1 and FIG. 3). However, either rIL-1α or rTNF-α augment the effect of rINF-γ on SM fibroblast CD40 expression (FIG. 3). rINF-γ also induces CD40 expression on SM fibroblasts that had lost CD40 expression during serial passages in culture (data not shown). Moreover, rINF-γ upregulates CD40 expression on dermal fibroblasts (FIG. 2). rIL-4 or rGM-CSF upregulate CD40 expression on B cells (25) or monocytes (12), respectively. However, rIL-4 or rGM-CSF have no effect on SM fibroblast CD40 expression (data not shown). Together, these studies demonstrate that rINF-γ induces and upregulates fibroblast CD40 expression and the addition of rIL-1α or rTNF-α augments this effect.

Effect of CD40L-CD40 Interactions on SM Fibroblast CD54 (ICAM-1) and CD106 (VCAM-1) Expression

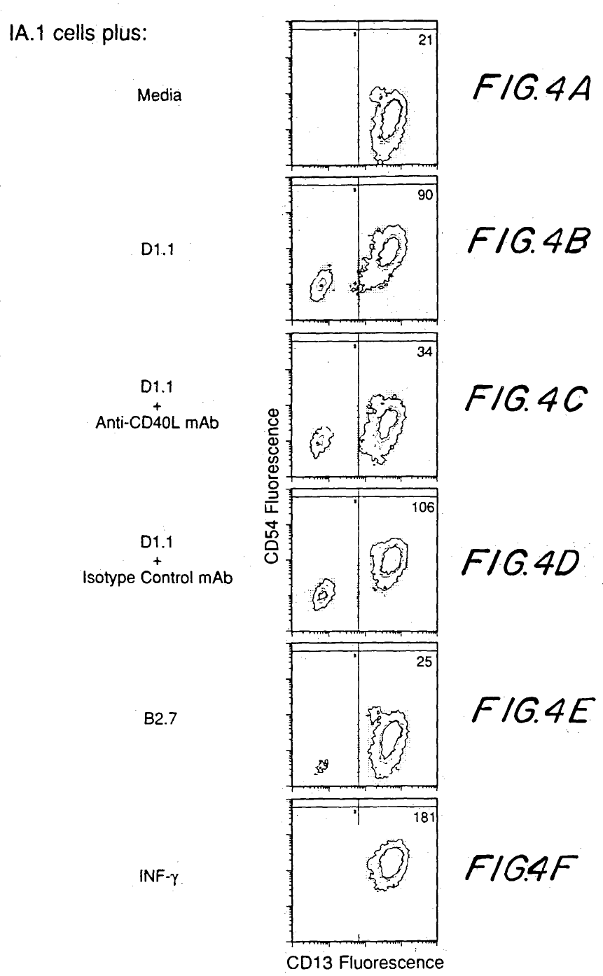

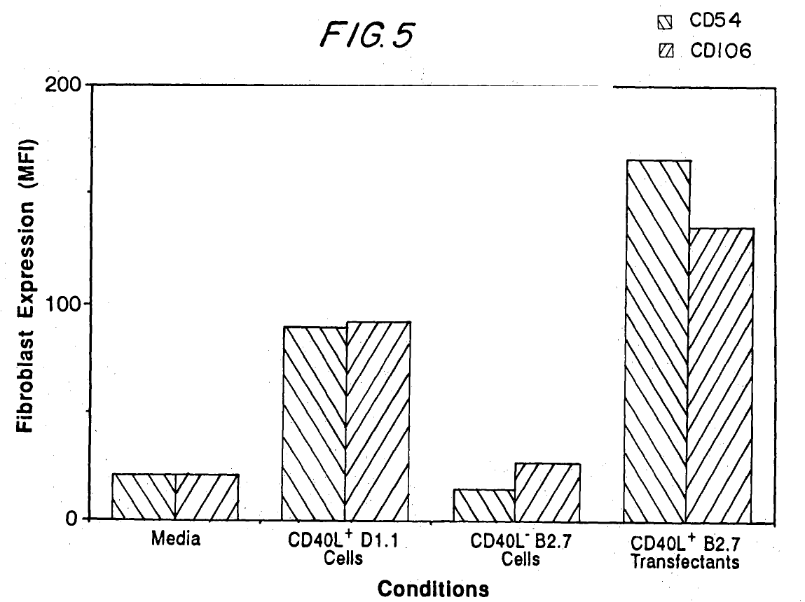

Because CD40 triggering is known to upregulate a variety of cell surface molecules on B cells, including adhesion molecules (26), it was determined if CD40 ligation upregulates CD54 or CD106 expression on SM fibroblasts. SM fibroblasts were cultured with CD40L⁺ Jurkat D1.1 cells in the presence or absence of anti-CD40L mAb 5C8 or control mAb. SM fibroblasts were also cultured with CD40L⁻ Jurkat B2.7 cells or CD40L⁺ Jurkat B2.7 transfectants. After the indicated period of time in culture, SM fibroblast CD54 or CD106 expression was determined by two-color FACS analysis. CD13 expression was utilized to discriminate SM fibroblasts from Jurkat T cells (27). CD40L⁺ D1.1 cells, but not control CD40L⁻ B2.7 cells, induce a 2-4 fold increase in SM fibroblast CD54 expression (FIGS. 4 and 5) in a manner that is specifically inhibited by mAb 5C8 but not by control mAb (FIG. 4). Moreover, CD40L⁺ D1.1 and CD40L⁺ Jurkat B2.7 transfectants, but not control CD40L⁻ B2.7 cells, similarly upregulate SM fibroblast CD106 expression (FIG. 5). Together, these results demonstrate that CD40L-CD40 interactions upregulate SM fibroblast CD54 and CD106 expression.

Effect of CD40 Ligation on SM Fibroblast IL-6 Secretion.

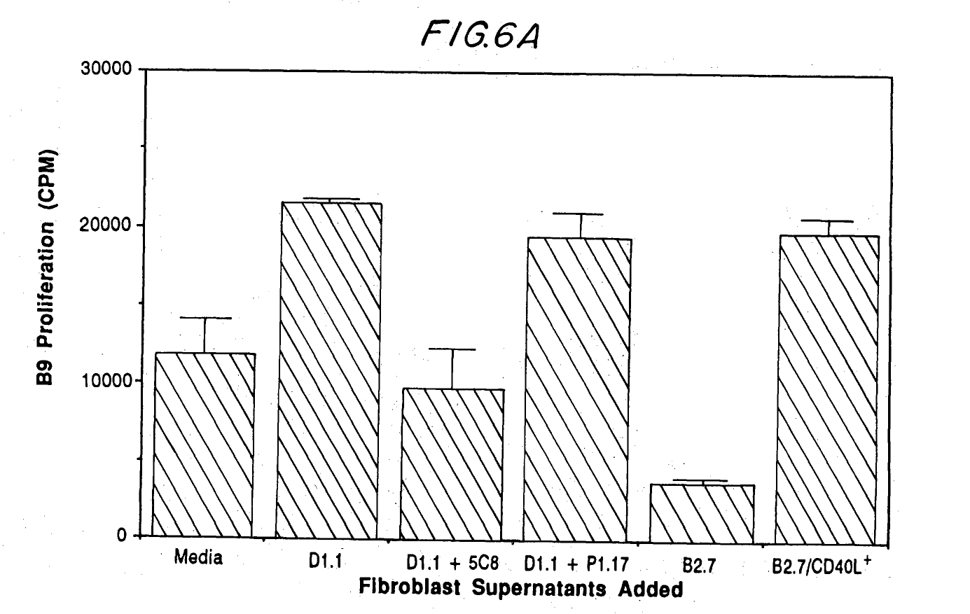

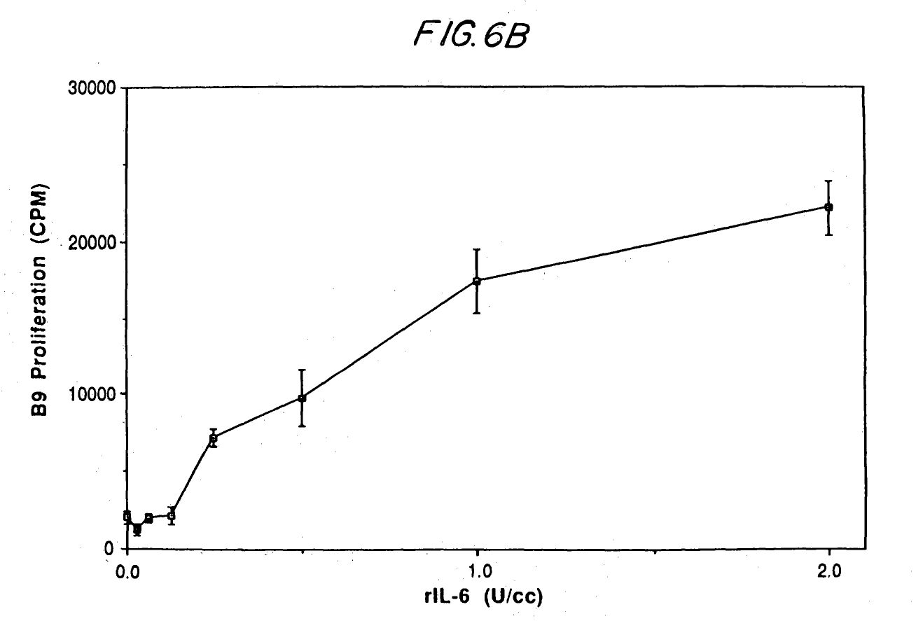

Ligation of CD40 induces B cells (28) and monocytes (12) to produce IL-6. Interestingly, SM fibroblasts produce IL-6 in vivo (29, 30) and in vitro (31). The next series of experiments asked if CD40L-CD40 interactions effect IL-6 secretion by SM fibroblasts. Therefore, SM fibroblasts were cultured with mitomycin-C treated CD40L⁺ Jurkat D1.1 cells in the presence or absence of anti-CD40L mAb 5C8 or control mAb. Additionally, SM fibroblasts were cultured with CD40L⁻ Jurkat B2.7 cells or CD40L+Jurkat B2.7 transfectants. Fibroblast supernatants or control supernatants from Jurkat cells cultured alone were collected after 48 hours, and dilutions added to the IL-6 responsive murine, B cell line B9. D1.1 cells and CD40L⁺ B2.7 transfectants, but not CD40L B2.7 cells, augment SM fibroblast IL-6 secretion (FIG. 6). Additionally, anti-CD40L mAb 5C8, but not control mAb, inhibits this effect of D1.1 cells. Control supernatants collected from Jurkat cells cultured alone did not induc B9 proliferation (See description of FIG. 6). These studies indicate that ligation of CD40 on SM fibroblasts augments IL-6 secretion.

Effect of CD40L-CD40 Interactions on SM Fibroblast Proliferation

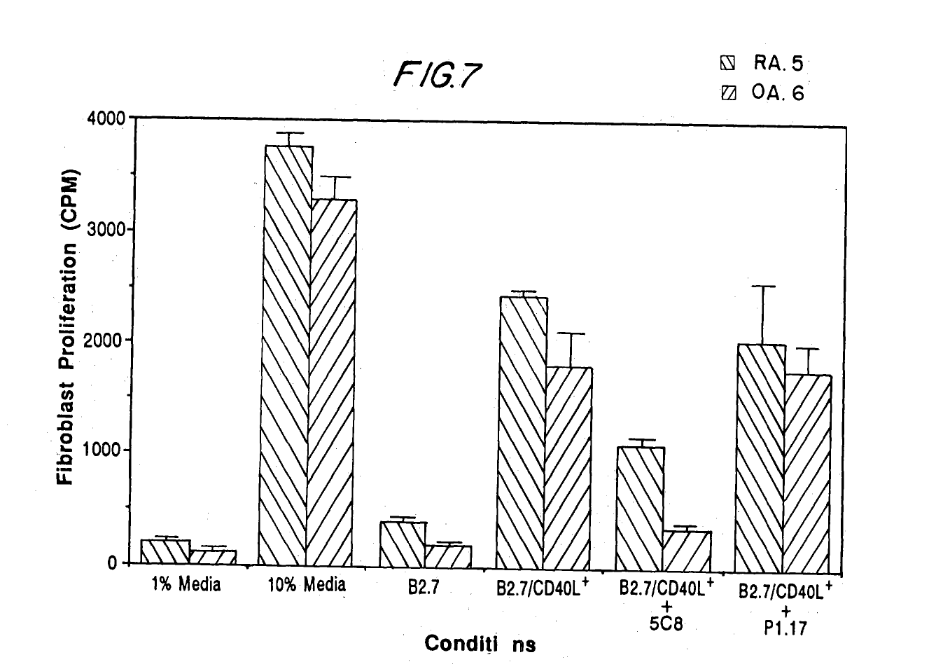

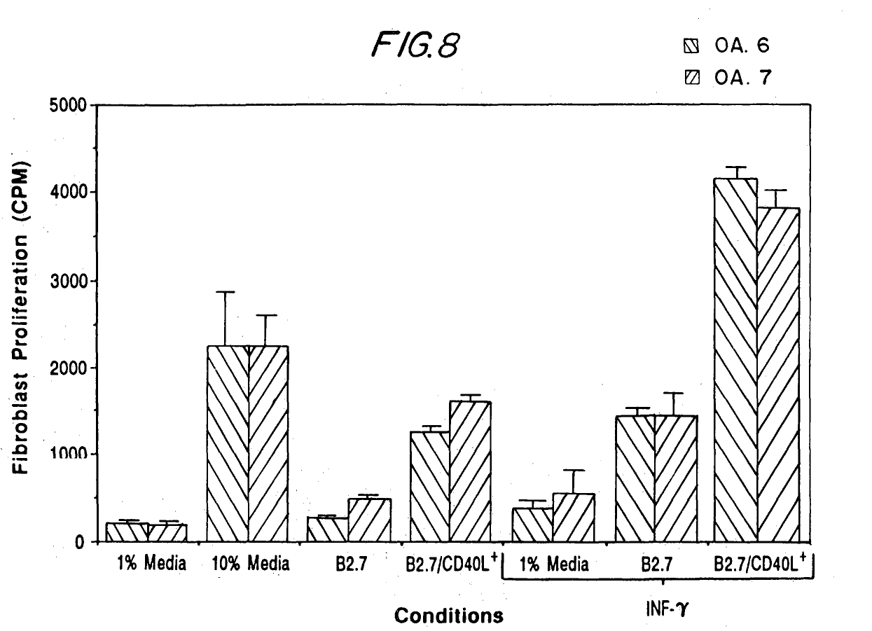

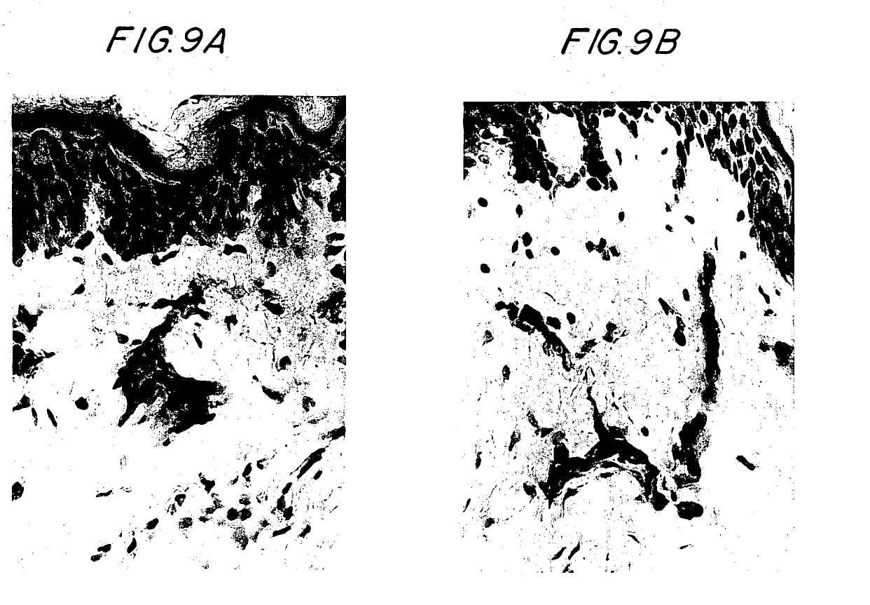

Because CD40 ligation induces B cell proliferation (5, 21), it was next asked if CD40L⁺ cells induce proliferation of SM fibroblasts. Therefore, SM fibroblasts were cultured overnight in 1% FM to arrest growth, as previously described (32), and further additions to the cells were performed in 1% FM, unless otherwise indicated. Mitomycin-C treated CD40L⁺ B2.7 transfectants or CD40L⁻ B2.7 cells were than added to the SM fibroblasts. Where indicated, co-culture experiments also included anti-CD40L mAb 5C8 or isotype control mAb P1.17. In some experiments, SM fibroblasts were pretreated overnight with rINF-γ prior to the addition of CD40L⁺ B2.7 transfectants. Because fibroblasts are known to proliferate in the presence of media containing 10% FCS ((32)), each experiment included control fibroblasts cultured in 10% FM. ³H thymidine incorporation was determined after 48 hours. CD40L⁺ B2.7 transfectants, in contrast to parental CD40L⁻ B2.7 cells, induce SM fibroblast proliferation (FIG. 7). Furthermore, anti-CD40L mAb 5C8 specifically inhibits the ability of CD40L⁺ B2.7 transfectants to induce fibroblast proliferation (FIG. 7). In addition, pretreatment of SM fibroblasts with rINF-γ augments the capacity of CD40L⁺ B2.7 transfectants to induce SM fibroblast proliferation (FIG. 8). Together, these data demonstrate that CD40L mediated signals induce SM fibroblast proliferation in vitro and this effect is enhanced by rINF-γ.

Discussion

This study extends current knowledge of CD40 expression and function by specifically demonstrating that: 1) cultured SM or dermal fibroblasts express cell surface CD40 molecules as determined by FACS analysis, 2) rINF-γ upregulates fibroblast CD40 expression and this effect is augmented by rIL-1α or rTNF-α. 3) CD40L-CD40 interactions upregulates SM fibroblast CD54 and CD106 expression, 4) ligation of CD40 augments SM fibroblast IL-6 production and 5) induces SM fibroblast proliferation. Together, these data demonstrate that CD40L-CD40 interactions functionally activate fibroblasts in vitro.

Several lines of evidence suggest that T cells modulate fibroblast functions in vivo. This is of importance because fibroblasts play reparative roles following tissue injury by producing extracellular matrix proteins. In addition, lymphocytes, macrophages and fibroblasts are the predominant cell types in granulomatous inflammatory reactions characteristic of certain infections. Moreover, T cells directly or indirectly mediate fibroblast activation and collagen deposition seen in diseases such as scleroderma or chronic graft versus host disease (33-35).

Animal models demonstrate that T cells modulate fibroblast function during host responses to tissue injury. In this regard, studies of wound healing show that wound strength and hydroxyproline content are significantly decreased by treating mice with cyclosporine A (36) or T cell depleting anti-Thy 1.2 mAb (37). T cells also modulate outcome in various animal models of fibrosis. For example, bleomycin-induced pulmonary fibrosis is significantly attenuated in athymic mice relative to control euthymic mice (38). Moreover, joint or liver inflammatory reactions and collagen deposition are also significantly reduced in athymic rats following intraperitoneal injection of streptococcal cell wall extracts (39, 40).

One study suggests that human fibroblasts can express CD40 in vivo. Potocnik and coworkers studied, the expression and distribution of various cell surface molecules, including CD40, on RA PBL, SF and SM (18). By immunohistochemistry they noted CD40 expression on a variety of cells in RA SM, including cells with spindle shape morphology suggestive of fibroblasts. SM fibroblasts are a predominant cellular component of the rheumatoid pannus. By producing collagenase, PGE2, IL-6. and other mediators, synovial fibroblasts are thought to be important contributors to the joint destruction characteristic of RA (30, 41-43). While electron microscopic studies have demonstrated direct T-fibroblast contact in rheumatoid synovial membrane (44), most studies have suggested that macrophage derived cytokines, such as IL-1 or TNF-α, activate fibroblasts (30). These studies suggest that direct contact mediated by CD40L-CD40 interactions also provides activation and proliferative signals to SM fibroblasts.

The mechanism by which CD40L mediated signals augment SM fibroblast proliferation is currently unknown. It is possible that CD40L-CD40 interactions induce the secretion of cytokines, such as IL-1, GM-CSF and FGF, which can stimulate SM fibroblast proliferation in an autocrine or paracrine manner (31). CD40 ligation also induces B cells to express c-myc (45) a proto-oncogene associated with proliferating cells. Immunohistologic studies demonstrate that RA SM fibroblast-like synoviocytes express c-myc in situ (46). Therefore, it will be of interest to specifically determine if CD40 ligation also induces c-myc expression in SM fibroblasts.

Similar to CD40 ligation on B cells (26), CD40L-CD4C interactions augment expression of fibroblast CD54 expression. In addition, CD40L-CD40 interactions upregulate fibroblast CD106 expression. CD54 and CD106 play key role in recruiting immune cells to sites of inflammation by interacting with CD11a/CD18 (LFA-1) or CD49d (VLA-4), respectively, expressed on leukocytes (24). There is also evidence that these ligand-counterligand interactions enhance proliferative signals to T cells (47). CD54 and CD106 are known to be expressed on RA fibroblast-like synoviocytes in vivo ((48-50)) and various cytokines upregulate synovial fibroblast CD54 and CD106 expression in vitro (49, 51, 52). Moreover, T cell adhesion to SM fibroblasts in vitro is partly mediated by CD11a/CD18-CD54 interactions (53) and CD49d-CD106 interactions (49). Therefore, CD54 and CD106 upregulation on SM fibroblasts by CD40L⁺ T cells may represent a mechanism to augment cytokine mediated inflammatory cell recruitment/retainment to SM. Additionally, CD40L mediated SM fibroblast CD54 and CD106 upregulation may play direct signaling roles to T cells via interactions with their counter-receptors.

It is of interest that in vivo administration of a hamster anti-murine CD40L mAb (MR1) prevents the induction of collagen-induced arthritis, a murine model of RA (54). The fact that MR1 blocks the production of anti-collagen autoantibodies likely relates to the known role of CD40L-CD40 interactions in T cell dependent humoral immune responses (9-11). Moreover, MR1 prevents the development of synovial lining cell thickening and SM inflammatory cell infiltration characteristic of collagen-induce arthritis (54). These studies suggest that T cell-fibroblast CD40L-CD40 interactions play roles in mediating inflammatory reactions seen in collagen-induced arthritis, an also plays immunopathogenic roles in human fibrotic diseases such as RA or scleroderma, mediated in part by T cell-dependent fibroblast activation. Moreover, this study provides new rational for blocking CD40L-CD40 interactions as therapy for human diseases mediated by CD4⁺ T cell induced fibroblast activation.

TABLE 1

| | | |
|---|---|---|
| OA.2 | OA.3 | IA.1 |

| Stimuli | CD40 | CD54 | CD40 | CD54 | CD40 | CD54 |
|---|---|---|---|---|---|---|
| Media | 18 | 129 | 76 | 134 | 47 | 120 |
| rINF-γ | 56 | 703 | 228 | 668 | 95 | 755 |
| rIL-1α | 22 | 286 | 82 | 304 | 37 | 292 |
| rTNF-α | 22 | 568 | 96 | 506 | 66 | 594 |

Table 1 Legend. Cytokine regulation of SM fibroblast CD40 expression. Shown is CD40 expression (mean fluorescence intensity) as determined by FACS analysis on the indicated SM fibroblast lines following coculture with media, rINF-γ (1000 U/ml), rIL-1α (10 pg/ml) or rTNF-α (200 U/ml). Background staining (MFI) of a control mAb is subtracted for each value.

Second Series of Experiments

Materials and Methods

Monoclonal Antibodies, Lectins and T Cell Lines

The IgG2a murine anti-CD40L mAb (5C8) was previously generated (20). Hybridomas W6/32 (anti-MHC Class I), L243 (anti-MHC. Class II), 3C10 (anti-CD14), THB.5 (anti-CD21), G28.5 (anti-CD40) and GAP 8.3 (anti-CD45) were purchased from American Type Culture Collection (ATCC) (Rockville, Md.). Hybridoma ascites was purified on a Protein G column (Pharmacia, Piscataway, N.J.). FITC conjugated anti-CD13, FITC conjugated-anti-CD19 and PE conjugated anti-CD54 mAbs was purchased from Biosource International (Camarillo, Calif.) and anti-CD34 mAb was obtained from Biogenex (San Ramon, Calif.). An additional anti-CD54 mAb, as well as anti-CD62E and anti-CD106 mAbs, were kindly provided by Biogen (Cambridge, Mass.). L243 and mAbs provided by Biogen were biotinylated as previously described (37). PE conjugated anti-CD80 and biotinylated, anti-CD86 mAbs were purchased from Becton Dickinson (San Jose, Calif.) and PharMingen (San Diego, Calif.), respectively. Isotype control mAbs utilized for FACS analysis were purchased from Becton Dickinson or Caltag Laboratories (South San Francisco, Calif.). P1.17 is an irrelevant control IgG2a murine mAb (Biogen) utilized for functional studies. FITC conjugated UEA-1 were obtained from Sigma (St. Louis, Mo.).

D1.1 is a Jurkat T cell subclone that constitutively expresses CD40L (20, 42). B2.7 is a CD40L⁻ Jurkat T cell subclone (20, 42). Stably transfected CD40L⁺ 293 kidney cells or CD8⁺ 293 kidney cells were generated as previously reported (37). Ramos 2G6 B cells respond to CD40L mediated signals (38, 39) and were obtained from ATCC.

Endothelial Cell Cultures

Human umbilical vein endothelial cells (HUVEC) were isolated as previously reported (40, 41). HUVEC were cultured in M199 media (Gibco, Grand Island, N.Y.) supplemented with 25% FCS (Summit Biotechnology, St. Collins, Colo.), 5% human serum (Gemini, Calabasas, Calif.), heparin 90 μg/ml, (Sigma), endothelial cell growth factor 15 μg/ml. (Collaborative Research, Bedford, Mass.) and 1% penicillin-streptomycin (Sigma) (M199 complete media). HUVEC were passaged by treatment for 3 minutes with 1% Trypsin-EDTA (Sigma). All HUVEC experiments were performed in M199 complete media following 1-3 passages.

Studies on the Effects of Cytokines on HUVEC CD40 Expression

To study the effects of cytokines on CD40 expression, HUVEC were cultured in 6 well plates (Nunc, Denmark) and grown to near confluence. The media was aspirated and HUVEC were then incubated with rIFN-γ 1000 U/ml (Biogen), rIL-1α 10 pg/ml (R & D, Minneapolis, Minn.) or rTNF-α 200 U/ml (Upstate Biotechnology, Lake Placid, N.Y.) in 3 ml of M199 complete media. At the indicated times, media was aspirated, cells were washed once with saline and 1 ml of 1% trypsin-EDTA was added to the wells. Cold Isocove's Modified Dulbecco's Media (Gibco) containing 10% FCS (Summit) was added to the wells after 3 minutes and the cells collected for FACS analysis.

Studies on Functional Consequences of HUVEC CD40 Ligation.

To study the effect of CD40 ligation on the expression of HUVEC cell surface molecules, cells were cultured in 6 well plates as described above. When. HUVEC were near confluence 1×10⁶ CD40L⁺ Jurkat D1.1 cells, CD40L⁻ Jurkat B2.7 cells, CD40L⁺ 293 kidney cell transfectants or CD8 kidney cell transfectants were added to the culture. Where indicated, CD40L⁺ cells were pretreated with anti-CD40L mAb 5C8 (10 μg/ml) or isotype control mAb P1.17 (10 μg/ml) prior to the addition to HUVEC. After the indicated time in culture the cells were collected by trypsinization and two-color FACS analyses performed.

Functional Studies of CD40 Ligation on Ramos 2G6 Cells.

Control experiments of CD40 ligation on Ramos 2G6 cells were performed by culturing 2×10⁵ Ramos 2G6 cells with 1×10⁵ D1.1 cells or control cells for 24 h hours in 96 well plates containing 200 μl of Isocove's Modified Dulbecco's Media (Gibco) containing 10% FCS (Summit) and 1% penicillin-streptomycin (sigma).

Cytofluorographic Analysis

The methods utilized for cytofluorographic analysis have been previously described (20, 42). In all experiments the cells were first treated with aggregated human immunbglobulin (Enzyme International, Fallbrook, Calif.) to block non-specific Ig binding. For single-color FACS analysis, cells were stained with saturating concentrations of primary antibody for 30-60 minutes at 4° C. Following washing, FITC conjugated F(ab)₂ goat anti-mouse IgG (Jackson Immunoresearch Laboratories, West Grove, Pa.) was added for 30-60 minutes at 4° C. The cells were washed and fixed with 1% formaldehyde prior to FACS analysis. For two-color FACS analysis, cells were first stained with the indicated biotinylated mAbs. Following washing, cells were then stained with streptavidin-PE (Calbiochem, La Jolla, Calif.) and FITC conjugated anti-CD13 mAb or FITC conjugated UEA-1, as indicated. HUVEC were distinguished from Jurkat cells in two-color FACS analysis by positive staining with anti-CD13 mAb or UEA-1, a lectin that selectively binds endothelial cells (43). Fluorescence intensity was measured on a FACScan cytofluorograph with the Consort-30 software (Becton-Dickinson, Mountainview, Calif.). Mean fluorescence intensity (MFI) refers to values normalized to the log scale as calculated by the Consort 30 software.

Characterization of Endothelial Cell CD40 Expression In Situ.

Frozen sections of normal spleen, thyroid, skin, muscle, kidney, lung or umbilical cord were studied for CD40 expression, as previously described (38). Immunohistologic analysis was performed with the indicated mAbs and reactivity detected using Vector ABC Elite kit and 3-amino-9-ethylcarbazole (AEC) (Vector Laboratories, Burlingame, Calif.) according to manufacture's instructions. Control frozen sections were stained with appropriate concentrations of mouse IgG (Sigma).

Results

In Situ and In Vitro Characterization of Endothelial Cell CD40 Expresssion.

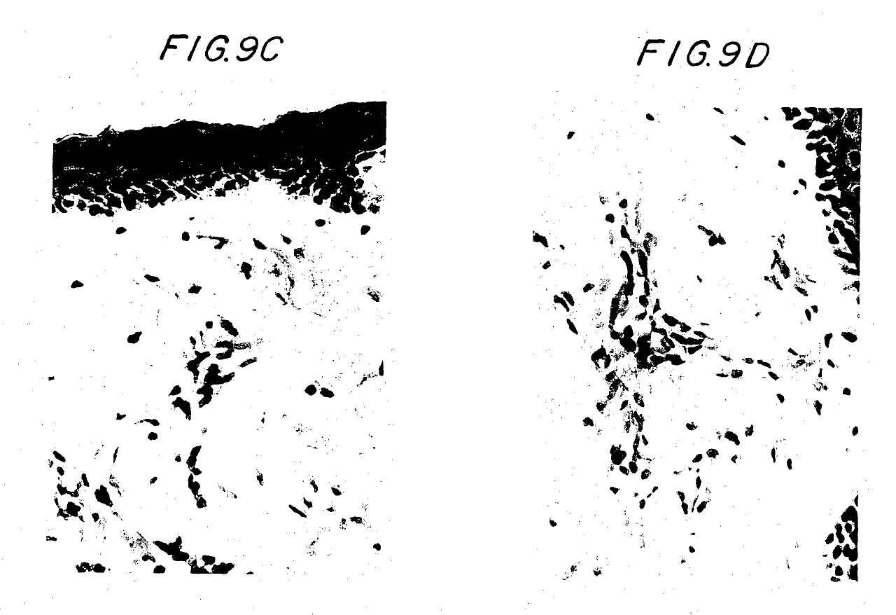

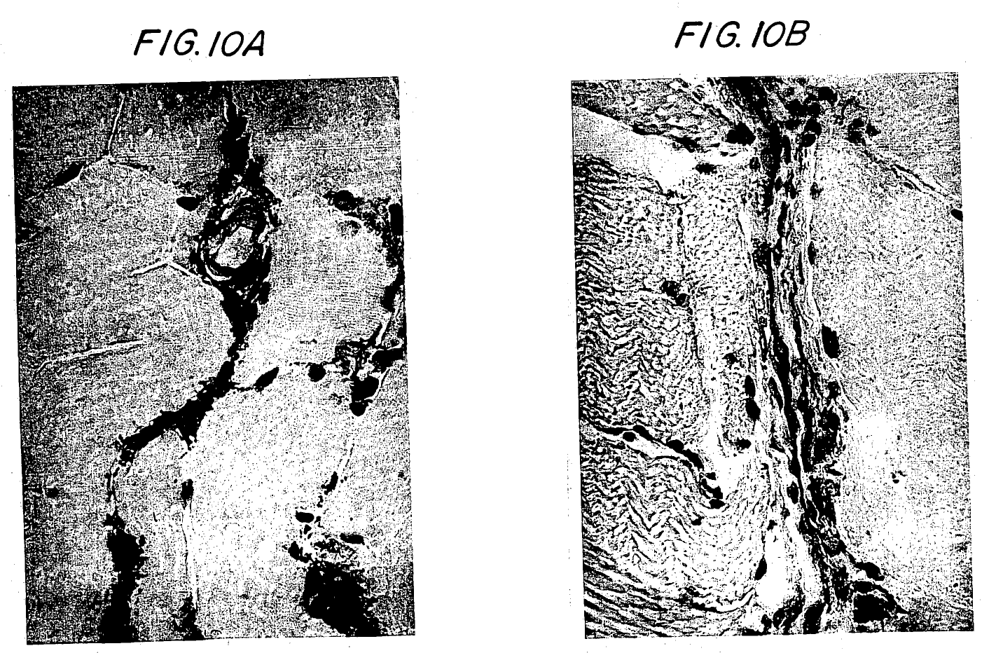

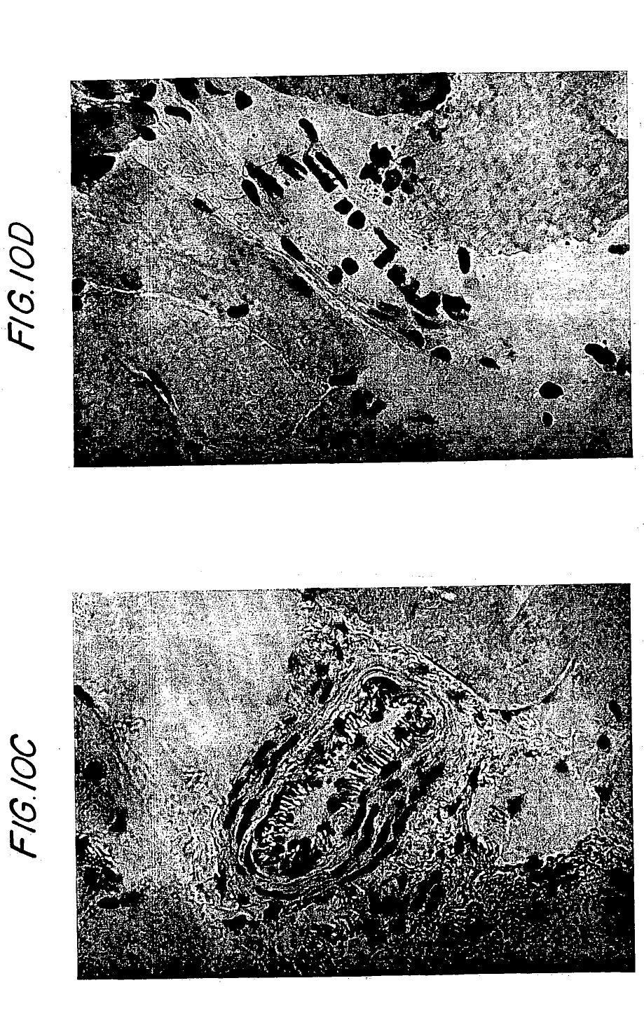

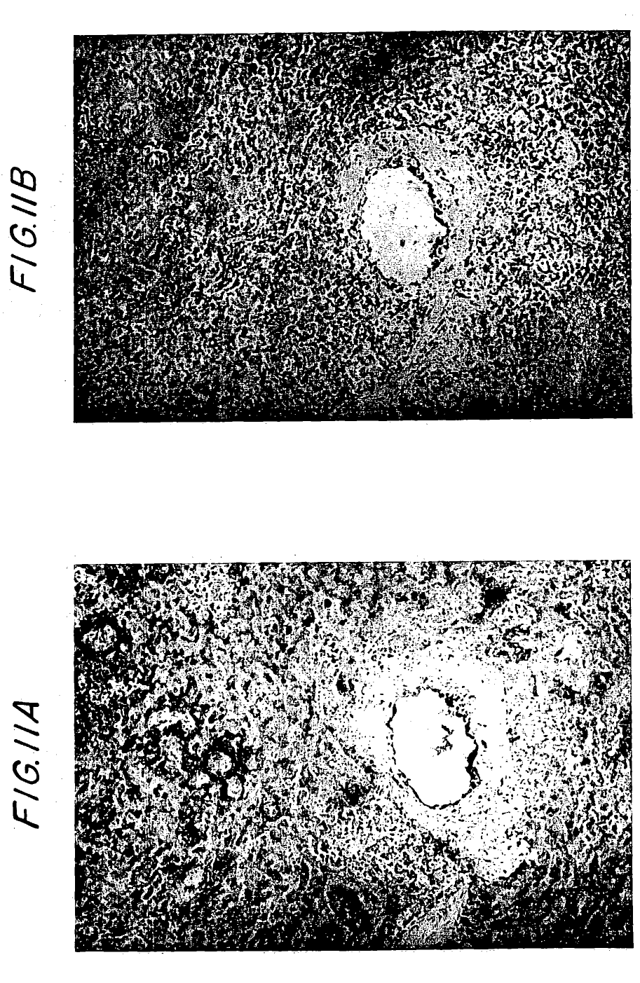

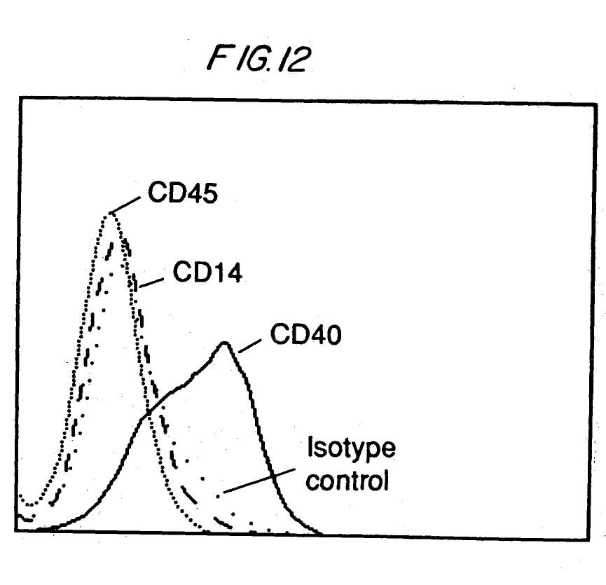

The first series of experiments were performed to determine if normal endothelial cells express CD40 in situ. Therefore, frozen sections obtained from normal spleen, thyroid, skin, muscle, kidney, lung or umbilical cord were stained with anti-CD40 mAb or control mouse IgG and endothelial cell reactivity noted. Additional controls included staining with anti-CD34 mAb (reactive with hematopoietic stem cells and endothelial cells (44)) or anti-CD21 mAb (reactive with B cell cells and epithelial cells (17)). Endothelial cells from all tissues studied express CD40 in situ. FIGS. 9-11 demonstrate representative CD40 staining of endothelial cells in normal skin (FIG. 9), muscle (FIG. 10) and spleen (FIG. 11). The pattern of endothelial reactivity was similar to that seen with anti-CD34 mAb (FIGS. 9 and 10). In contrast, endothelial cells did not react with anti-CD21mAb (FIGS. 9 and 10) or mouse IgG (FIGS. 9-11).

To further explore endothelial cell CD40 expression and function in vitro it was next asked if cultured human umbilical vein endothelial cells (HUVEC) also express CD40. HUVEC were isolated, grown to confluence and CD40 expression determined by FACS analysis following trypsinization. The cells morphologically resembled endothelial cells and phenotypic analysis demonstrated that the cells were CD13⁺ and reactive with UEA-1, a lectin that selectively binds endothelial cells (43). In addition, the cells were CD14⁻ CD45⁻MHC Class-II⁻ by FACS analysis. Therefore, these cultures did not contain significant numbers of contaminating non-endothelial cells. HUVEC constitutively express CD40 in vitro (FIG. 12). Similar results were obtained from HUVEC isolated from 15 individuals.

To determine if pro-inflammatory cytokines regulate endothelial cell CD40 expression, as has been shown for B cells (45), monocytes (14), thymic epithelial cells (18) and fibroblasts (19), HUVEC were cultured with rIFN-γ, rIL-1α, or rTNF-α for 48 hours. rINF-γ, in contrast to rIL-1α or rTNF-α, induces 2-3 fold increase in HUVEC CD40 expression (table 2). Together, these studies demonstrate that endothelial cells from normal tissue express CD40 in situ and in vitro and that rIFN-γ upregulates endothelial cell CD40 expression in vitro.

Effect of CD40L-CD40 Interactions on HUVEC CD54, CD62E and CD106 Expression.

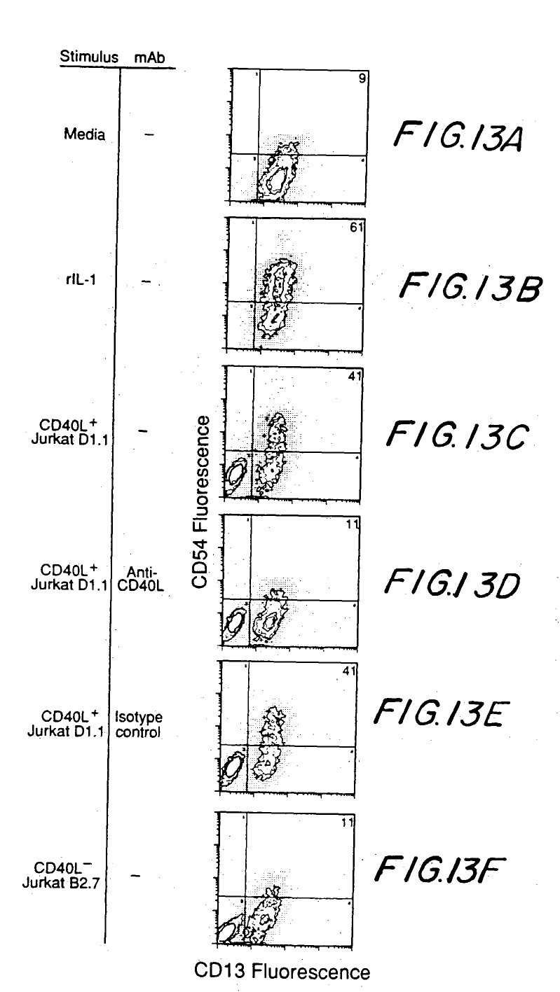

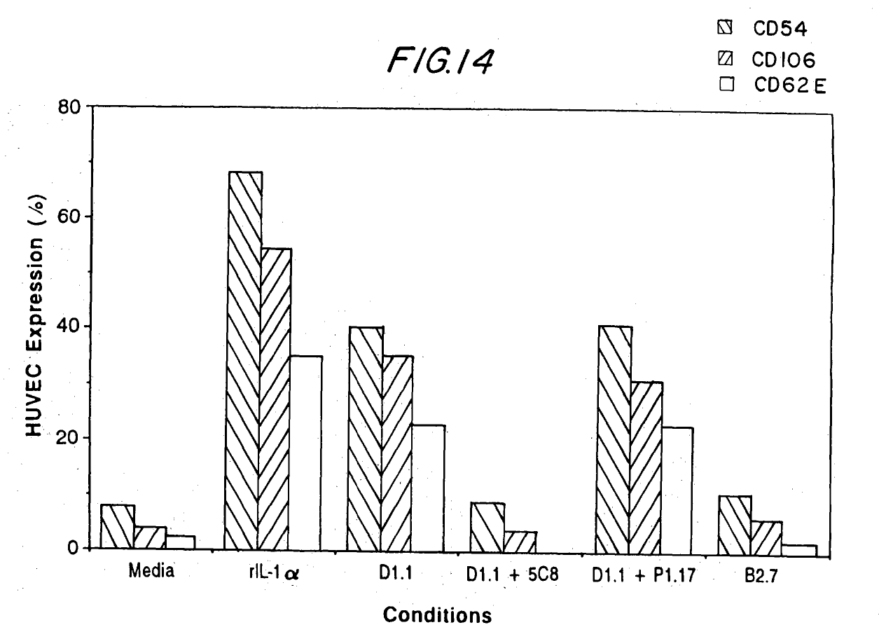

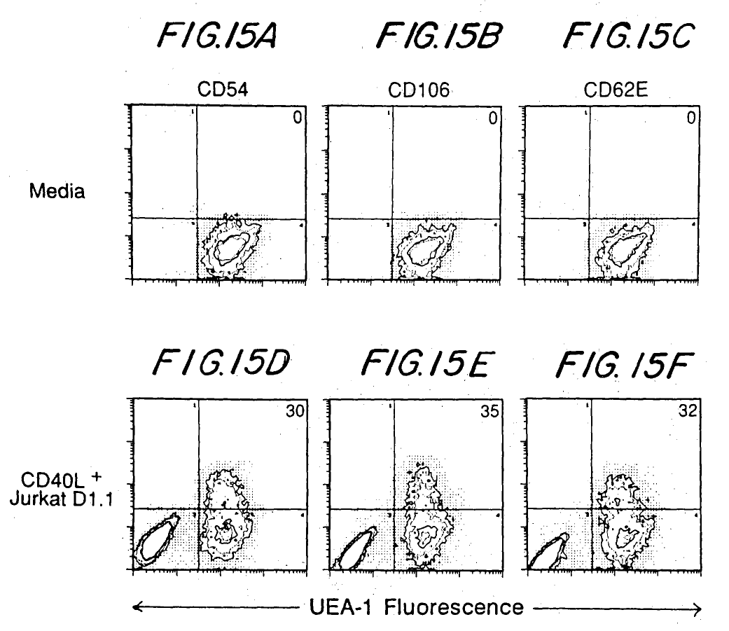

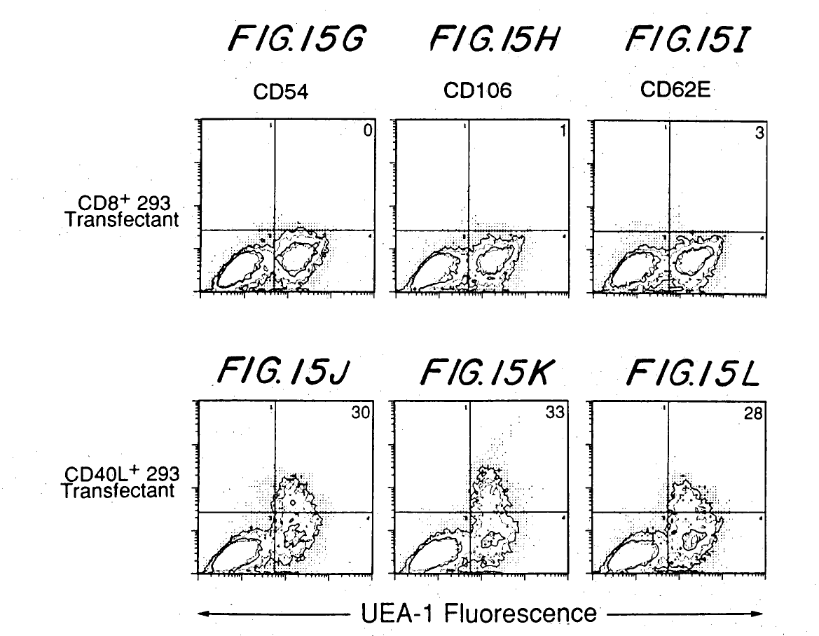

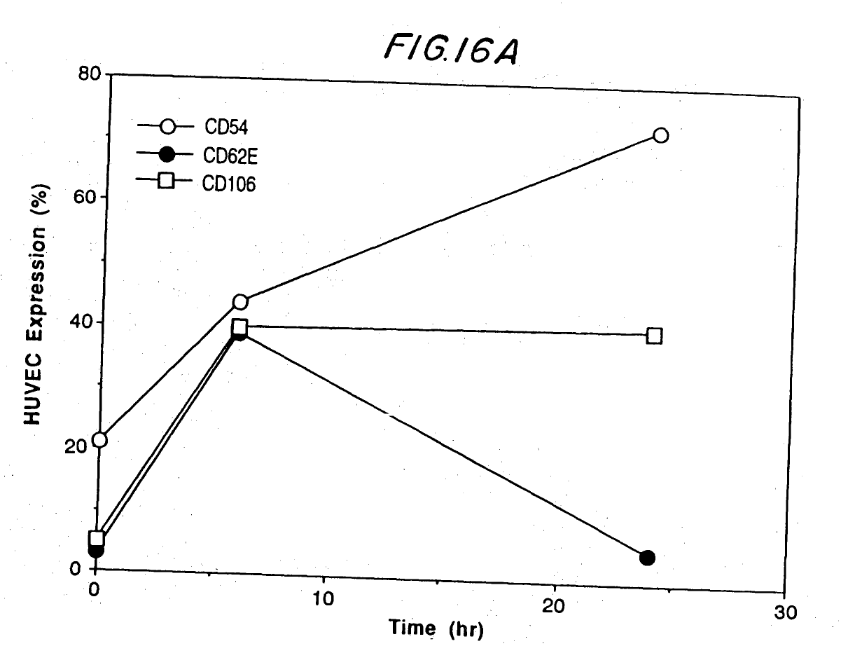

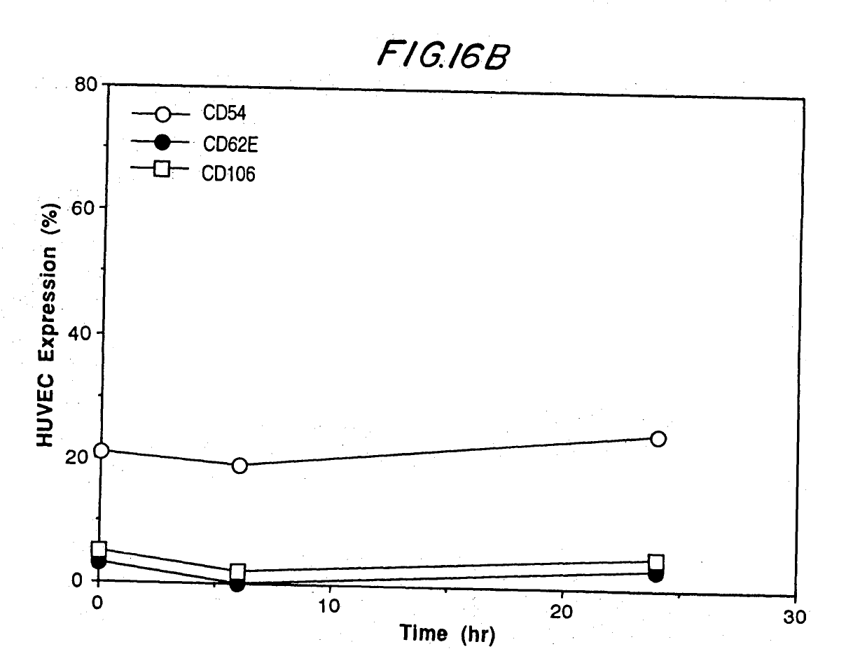

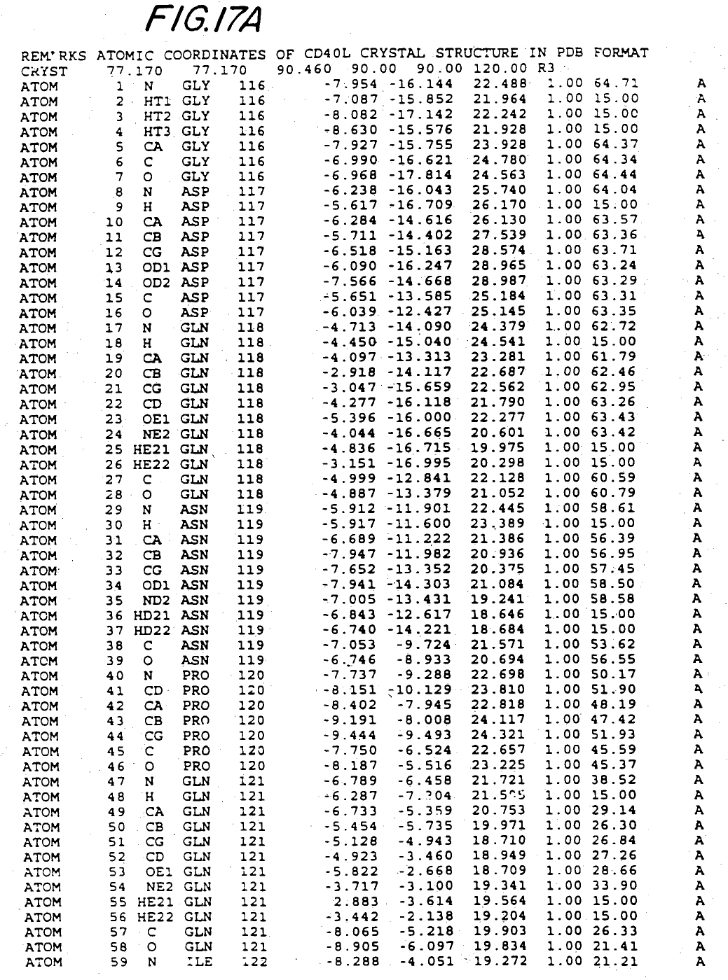

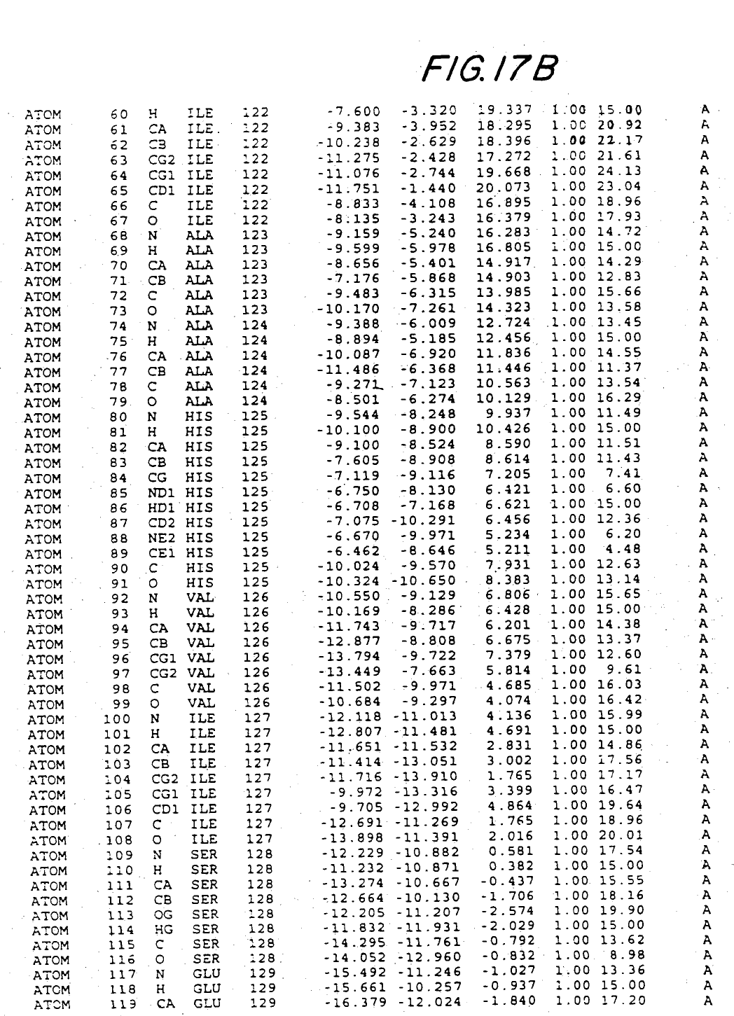

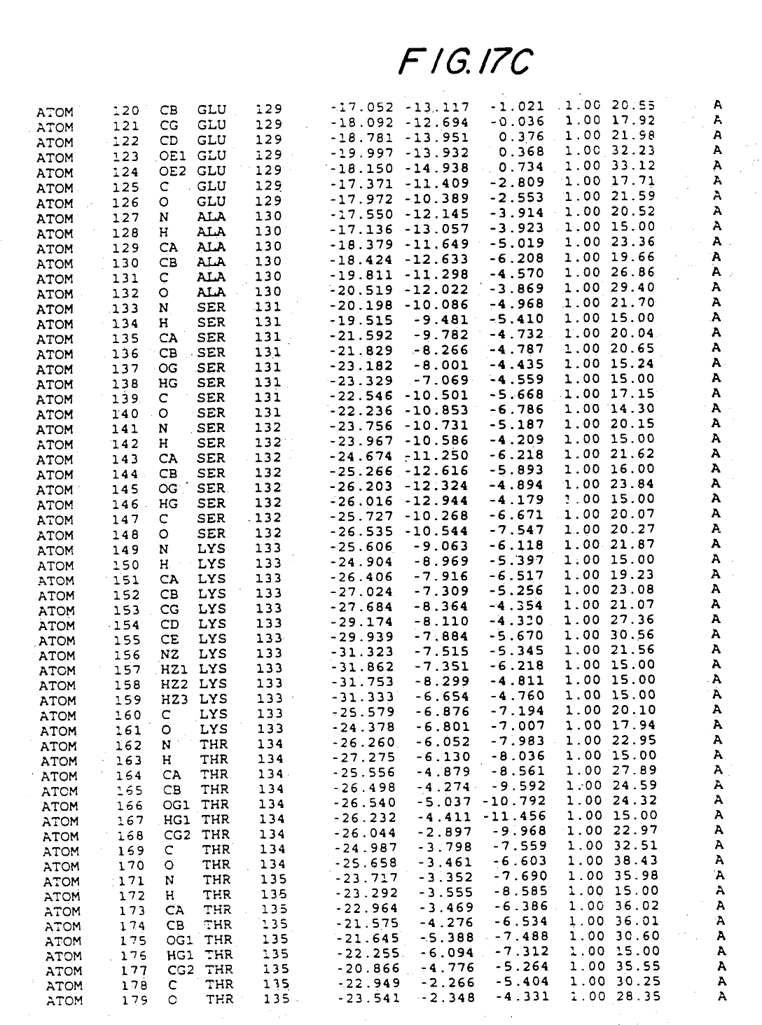

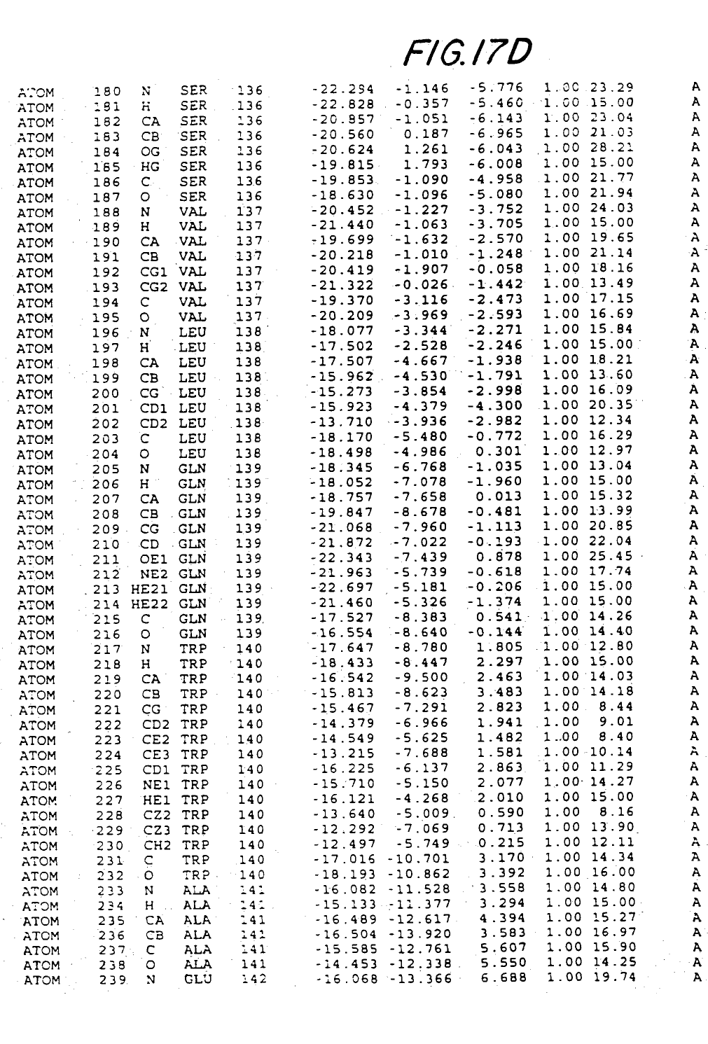

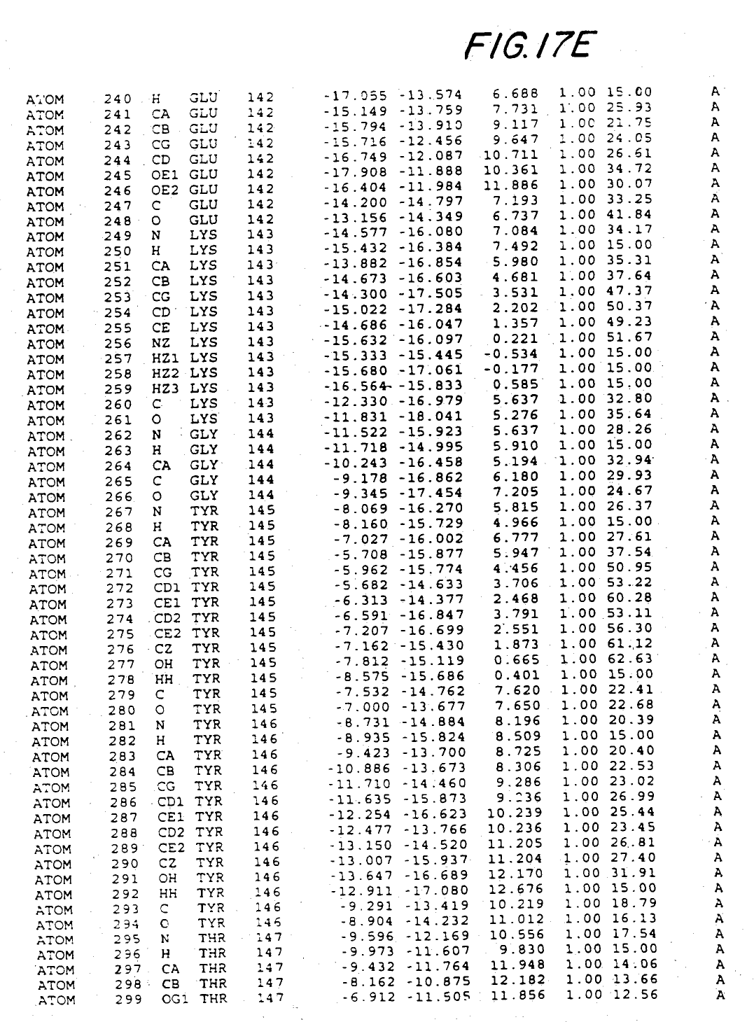

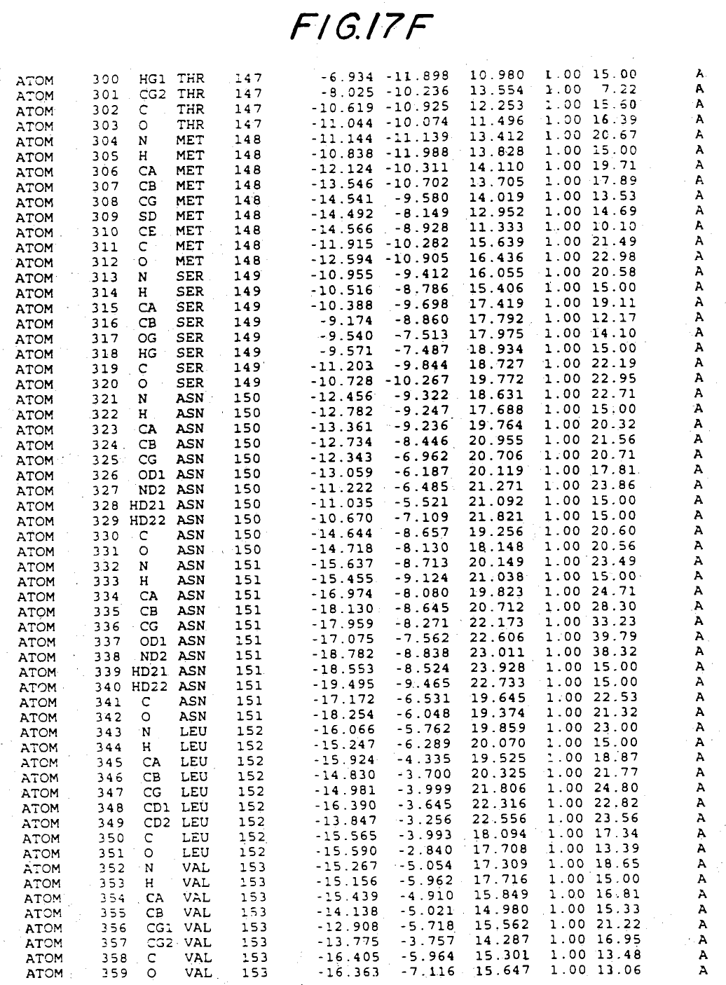

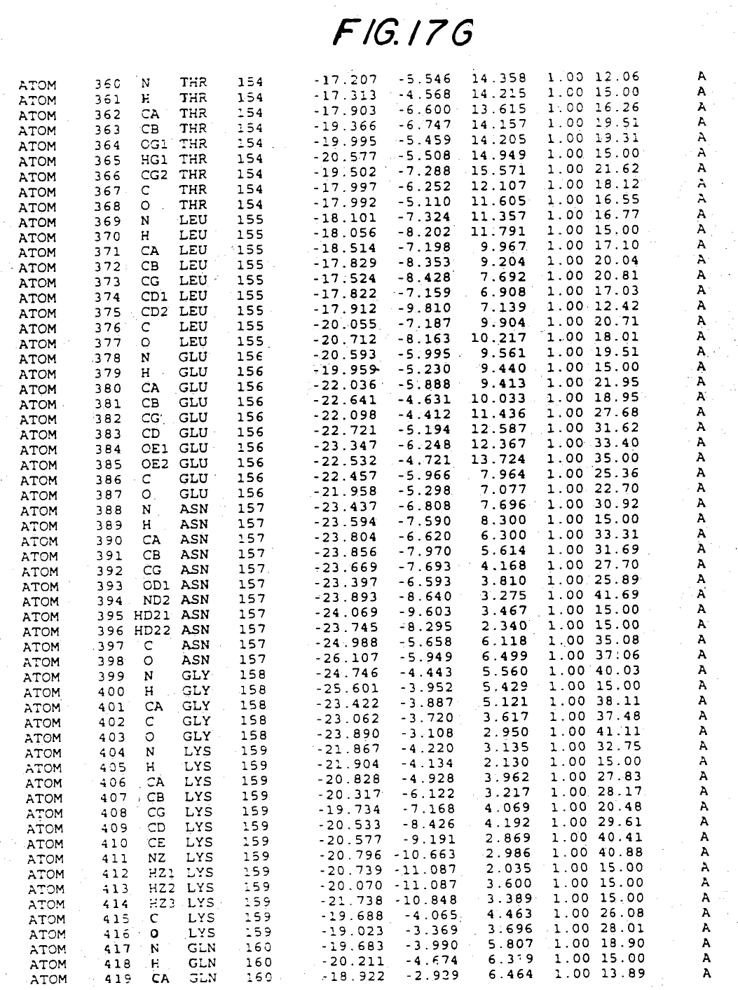

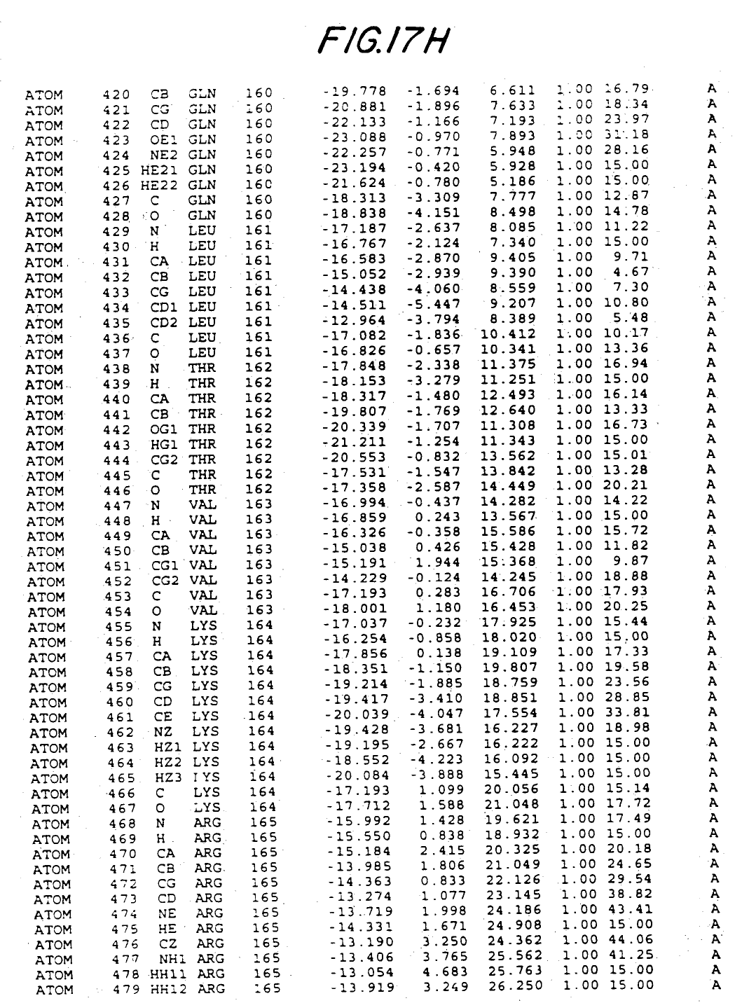

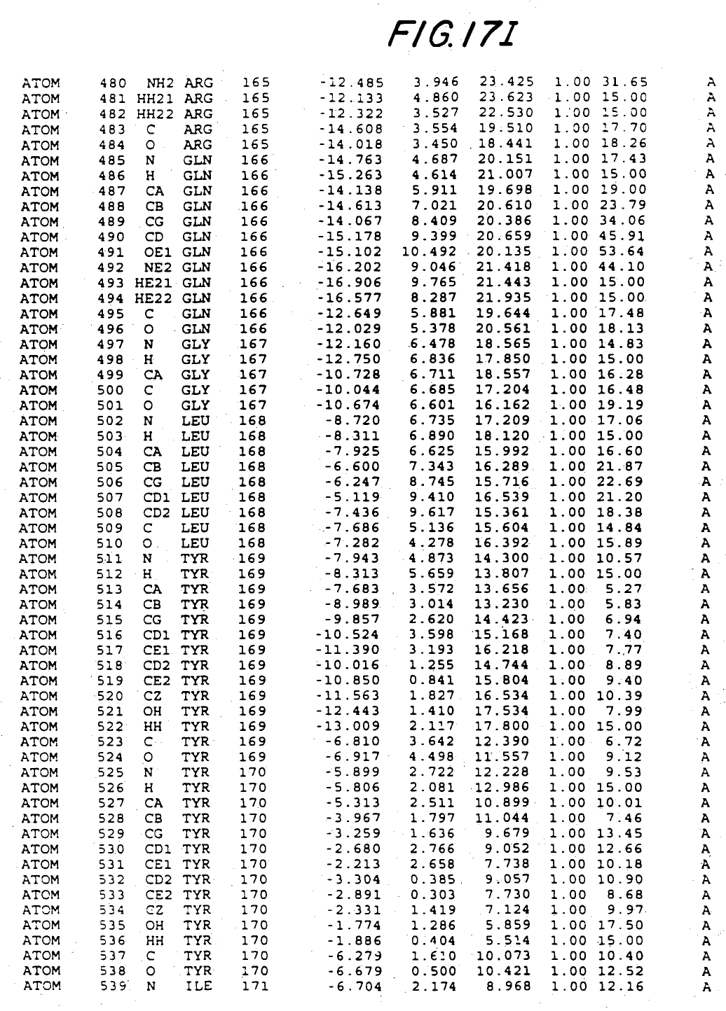

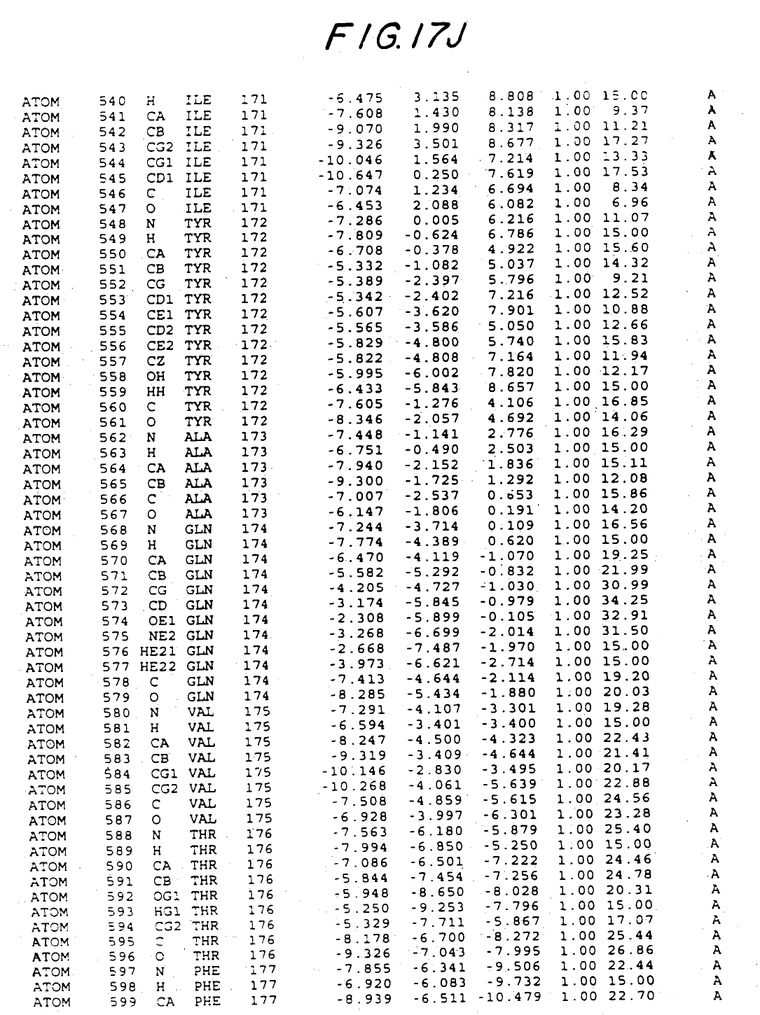

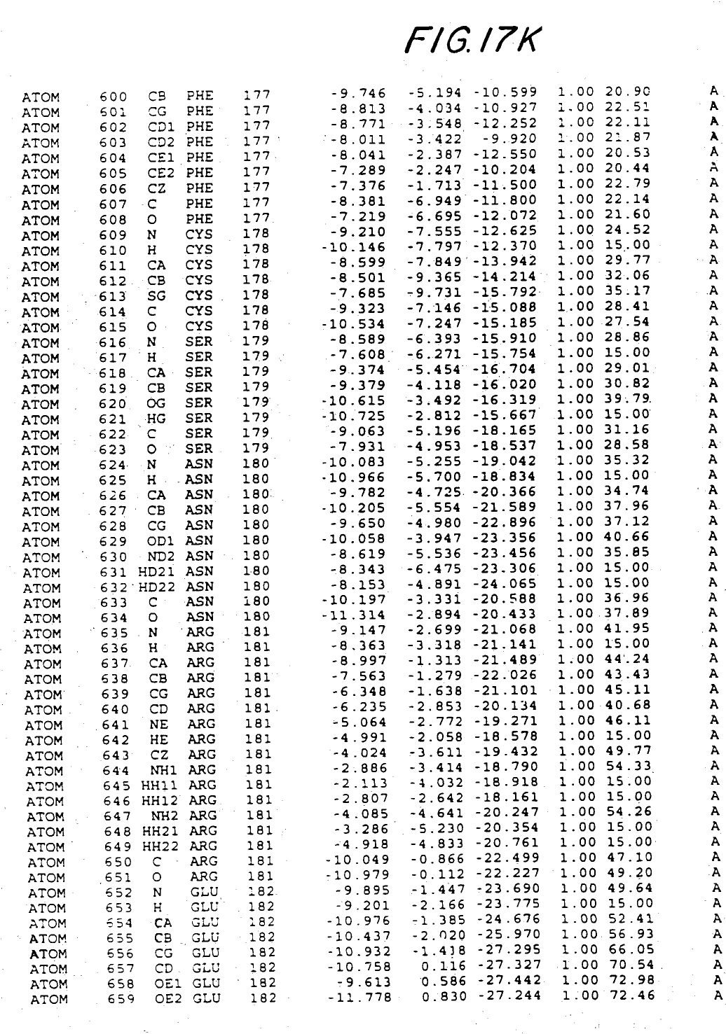

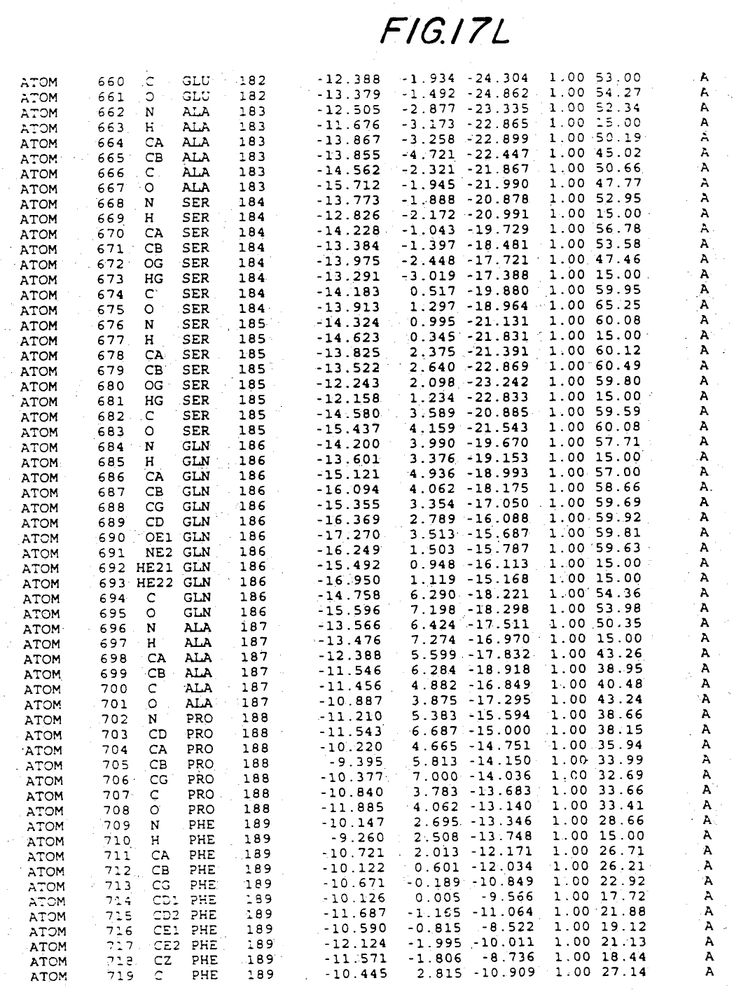

Activated endothelial cells express cell surface molecules, such as CD54, CD62E and CD106 that play important roles in mediating intercellular adhesive interactions (1, 2). Interestingly, ligation of CD40 on B cells (46) or fibroblasts (19) induces the upregulation of adhesion molecules. Therefore, it was next asked if CD40L-CD40 interactions effect the expression of CD54, CD62E or CD106 expression on HUVEC in vitro as determined by two-color FACS analysis. HUVEC were cultured with CD40L⁺ Jurkat D1.1 cells or CD40L⁻ Jurkat B2.7 cells. Where indicated, Jurkat D1.1 cells were pretreated with anti-CD40L mAb 5C8 or control mAb prior to the addition to HUVEC. As a positive control, HUVEC were-also cultured with rIL-1α. CD40L⁺ Jurkat D1.1 cells, but not CD40L⁻ Jurkat B2.7 cells, induce CD54, CD62E and CD106 upregulation on HUVEC (FIGS. 13 and 14). This effect of D1.1 cells is inhibited by anti-CD40L mAb 5C8 but not by an isotype control mAb (FIGS. 13 and 14). These studies strongly suggest that CD40L-CD40 interactions upregulate CD54, CD62E-and CD106 expression on HUVEC.

Effect of CD40L⁺ 293 Kidney Call Transfectants, on HUVEC CD54 CD62E and CD106 Expression.

To determine,if CD40L mediated signals were sufficient, in the absence of additional lymphoid specific interactions, to upregulate endothelial cell adhesion molecules, HUVEC were cultured with stably transfected CD40L⁺ 293 kidney cells or control CD8⁺ 293 transfectants. As a positive control, HUVEC were also cultured with CD40L⁺ D1.1 cells. Similar to CD40L⁺ D1.1 cells, CD40L⁻ 293 kidney cell transfectants upregulate CD54, CD62E and CD106 expression on HUVEC (FIG. 15). Control 293 CD8 transfectants have no effect on HUVEC CD54, CD62E or CD106 expression. Together, these studies demonstrate that CD40L-CD40 interactions are sufficient to upregulate these adhesion molecules on HUVEC in vitro.

Analysis of the Kinetics of CD40L Mediated HUVEC CD54, CD62E and CD106 Upregulation.

The kinetics of CD54, CD62E or CD106 upregulation by rIL-1α or rTNF-α in vitro has been well established (1, 2). CD54 and CD106 are upregulated 6 hours following activation and expression persist for greater than 24 hours. In contrast, CD62E expression peaks 6 hours following activation and returns to baseline (no expression) by 24 hours. In the next series of experiments the kinetics of CD40L induced HUVEC CD54, CD62E or CD106 upregulation were determined. HUVEC were cultured with CD40L⁺ D1.1 cells or CD40L B2.7 cells and analyzed at various time points for CD54, CD62E or CD106 expression. Following culture with CD40L⁺ D1.1 cells, HUVEC CD54 or CD106 expression was upregulated by 6 hours and persisted in expression for greater than 24 hours (FIG. 16). In contrast, CD40L induced CD62E expression peaked by 6 hours and returned to baseline by, 24 hours (FIG. 16). Therefore, the kinetics of CD40L, rTNF-α or rIL-1α mediated upregulation of HUVEC CD54, CD62E or CD106 are similar.

Determining if CD40L-CD40 Interactions Upregulate CD80, CD86 or MHC Class II Expression on HUVEC

Activated endothelial cells are competent to express MHC Class II molecules and deliver costimulatory signals to T cells (10, 47-49). Ligation of CD40 on B cells or dendritic cells upregulates MHC Class II expression, as well as, the expression of the costimulatory molecules CD80 and CD86 (36, 37, 50-52). Therefore the next series of experiments determined if CD40L-CD40 interactions similarly upregulates. MHC Class II, CD80 or CD86 expression on HUVEC. HUVEC were cultured with CD40L⁺ D1.1. cells or CD40L⁻ B2.7 cells for 24 or 48 hours and CD80, CD86 and MHC Class II expression determined by two-color FACS analysis. As a positive control for the effect of HUVEC CD40 ligation, CD54 expression was also determined. In addition, HUVEC were also cultured with rIFN-γ as a control for MHC Class II upregulation. As a positive control for CD40L mediated CD80, CD86 and MHC Class II upregulation, D1.1 cells were cultured with Ramos 2G6 B cells (38-39). In contrast to the effects of CD40 ligation on B cells or dendritic cells, CD40L-CD40 interactions do not upregulate MHC Class II, CD80 or CD86 expression on HUVEC (table 3).

Discussion

CD40is a cell surface molecule constitutively expressed on a variety of cells, including B cells (12, 13), monocytes (14), dendritic cells (15), epithelial cells (17, 18), basophils (16) and fibroblasts (19). The counter-receptor for CD40 is CD40L, a 30-33 kDa activation-induced, transiently expressed CD4⁺ T cell surface molecule (20-25). It is shown that endothelial cells in spleen, thyroid, skin, muscle, kidney, lung or umbilical cord express CD40 in situ. This finding is consistent with a previous report that endothelial cells in rheumatoid arthritis synovial membrane express CD40 (11). In addition, human umbilical vein endothelial cells (HUVEC) express CD40 in vitro. Most importantly, CD40 expression on endothelial cells is functionally significant because CD40L⁺ Jurkat T cells or CD40L⁺ 293 kidney cell transfectants, but not control cells, upregulate the expression of intercellular adhesion molecules CD54 (ICAM-1), CD62E (E-selectin) and CD106 (VCAM-1) on HUVEC. The results disclosed herein demonstrate that endothelial cells express CD40 and CD40L-CD40 interactions induce endothelial cell activation in vitro.

Endothelial cells play central roles in inflammatory responses in part by expressing CD54, CD62E and CD106 (1, 2). These adhesion molecules interact with specific cell surface receptors on leukocytes and promote the transmigration of inflammatory cells across the endothelial cell barrier. The expression of these particular endothelial cell surface molecules are tightly regulated (1, 2). Resting endothelial cells express low levels of CD54 and minimal or no CD62E or CD106. However, endothelial cells upregulate CD54, CD62E and CD106 expression following activation with IL-1 or TNF. These findings demonstrate a means by which activated CD4⁺ T cells upregulate endothelial cell adhesion molecules by direct cell-cell contact.

Because CD40L expression is also tightly regulated, it is likely that CD40L-CD40 interactions occur during Ag driven immune responses. In this regard, in vitro studies demonstrate that resting CD4⁺ T cells do not express detectable CD40L (20-22, 25, 53). However, CD40L is transiently expressed on activated CD4⁺ T cells in vitro; peak expression is seen 6 hours following activation and levels return to baseline (no expression) by 24-48 hours (20, 21, 53). CD40L is also rapidly down-modulated by CD40 expressing cells in a process that is at least partly due to receptor-mediated endocytosis (54). In vivo, CD40L expression is normally restricted to CD4⁺ T cells in secondary lymphoid tissue (38), the site of MHC restricted, Ag specific T—B interactions. However, immunohistologic studies of rheumatoid arthritis synovial membrane or psoriatic plaques demonstrates the presence of CD40L⁺CD4⁺ T cells. These studies suggest that APCs at sites of inflammation induce infiltrating CD4⁺ T cell to express CD40L. CD40L⁺CD4⁺ T cells then play roles in augmenting the inflammatory process by interacting with CD40⁺ endothelial cells. The functional consequences of this interaction enable further adhesion and transmigration of immune cells at sites of inflammation.

The fact that CD40 ligation regulates the expression of endothelial cell surface adhesion molecules is consistent with a general role for CD40 signalling in regulating the expression and/or function of adhesion molecules on a variety of cells. In this regard, it has been shown that CD40L mediated signals induce CD54 and CD106 upregulation on fibroblasts cultured from synovial membrane (19). CD40 ligation also upregulates CD54 expression on B cells (46) and induces CD54 dependent homoaggregation of B cells (55). Interestingly, pretreatment of B cells with anti-CD40 mAb augments heterotypic interactions of B cells with activated endothelial cells in vitro in a manner dependent on CD49d (VLA-4)/CD106 interactions (56). Because CD40 ligation did not upregulate B cell CD49d expression, it was hypothesized that CD40 mediated signals induced CD49d activation.

CD40 ligation on B cells or dendritic cells also upregulates expression of MHC Class II, as well as, the costimulatory molecules CD80 and CD86 (36, 37, 50-52). Interestingly, endothelial cells stimulated with rIFN-γ are competent, to express MHC Class II in vitro (57) and endothelial cells in situ within inflammatory tissue can express MHC Class II (10, 58-60). Moreover, endothelial cells are competent to present Ag to T cells in vitro and deliver appropriate costimulatory signals to T cells required for IL-2 production and proliferation (10, 47-49).

However, it is shown here that CD40L-CD40 interactions do not, upregulate MHC Class II, CD80 or CD86 expression on HUVEC in vitro. This finding is consistent with previous studies suggesting that human endothelial cells do not express CD80 (47, 61). The costimulatory molecules expressed on endothelial cells are not precisely known. Work by Pober and colleagues demonstrate that blocking CD2-CD54 (LFA-3) interactions inhibits the ability of endothelial cells to induce alloqenic T cell proliferation (47-48). However, it is unclear if CD2-CD58 interactions enhance intercellular adhesiveness and/or deliver costimulatory signals to T cells. It will be of interest to determine if CD40L mediated signals modulate the capacity of endothelial cells to activate T cells.

Finally, endothelial cells are activated in a variety of diseases mediated by CD4⁺ T cells. For example, endothelial cell surface adhesion molecules are upregulated in rheumatoid arthritis (62), scleroderma (63) and in transplant rejection (64). In addition, CD4⁺ T cells play roles in atherosclerosis (65) and accelerated atherosclerosis associated with transplantation (60). The precise mechanistic role of CD40L mediated interactions with endothelial cells in these diseases is not known. However, an antibody to CD40L, MR1, inhibits murine models of diseases mediated by CD4⁺ T cells and/or inflammatory cell infiltrates. For example, MR1 prevents the synovial lining cell hypertrophy and cellular infiltrate associated with collagen-induce arthritis, a murine model of rheumatoid arthritis (66). Moreover, MR1 inhibits a murine model of multiple sclerosis (EAE) and inhibits allograft rejection (67). Blocking CD40L dependent interactions with endothelial cells and/or fibroblasts mediates, in part, these effects of MR1. The results disclosed herein suggest that CD40L-CD40 interactions on the surface of endothelial cells play immunopathogenic roles in inflammatory diseases.

TABLE 2

HUVEC Expression

| Stimuli | CD40 (MFI) | CD54 (MFI) |
|---|---|---|
| Media | 17 | 22 |
| rINF-γ | 42 | 44 |
| rIL-1α | 24 | 51 |
| rTNF-α | 22 | 54 |

Table 2 Legend. Effect of cytokines on HUVEC CD40 expression. Shown is the mean fluorescence intensity (MFI) of CD40 or CD54 expression on HUVEC cultured in the presence or absence of rIFN-γ (1000 U/ml), rIL-1α (10 pg/ml) or rTNF-α (200 U/ml) for 48 hours. CD40 or CD54 MFI was determined by FACS analysis and background staining of control mAb is subtracted for each value. Similar results were obtained in 2 additional experiments with different HUVEC lines.

TABLE 3

| | HUVEC Expression (MFI) | | | | Ramos Expression (MFI) | | | |
|---|---|---|---|---|---|---|---|---|
| Conditions | CD54 | CD80 | CD86 | MHC II | CD54 | CD80 | CD86 | MHC II |
| Media | 8 | 0 | 1 | 0 | 22 | 0 | 7 | 128 |
| D1.1 | 78 | 0 | 0 | 0 | 71 | 8 | 13 | 223 |
| B2.7 | 23 | 0 | 1 | 1 | 25 | 1 | 7 | 127 |
| rIFN-γ | 16 | 0 | 0 | 97 | ND | ND | ND | ND |

Table 3 Legend. Effect of CD40L-CD40 interactions on HUVEC MHC,Class II, CD80 and CD86 expression. Shown is the mean fluorescence intensity of HUVEC CD54, CD80, CD86 or MHC Class II expression following culture with media, rIFN-γ (1000 U/ml), CD40L⁺ Jurkat D1.1 cells or CD40L B2.7 cells for 48 hours. In a parallel experiment, the CD40L responsive Ramos 2G6 B cell line (38-39) was cultured with media, CD40L⁺ Jurkat D1.1 cells or CD40L B2.7 cells for 24 hours. HUVEC or Ramos 2G6 MHC Class II, CD54, CD80 and CD86 expresssion was determined by two-color FACS analysis. Background staining of control mAb is subtracted for each value. Shown is representative of 3 similar experiments with different HUVEC lines. ND=not done.

References for Background
1. Pauli, S., Ehlin-Henriksson, B., Mellstedt, H., Koho, H., Ben-Aissa, H. Perlmann, P. (1985) A p50 surface antigen restricted to human urinary bladder carcinomas and B lymphocytes. Cancer Immunol. Immunother. 20, 23-28.
2. Clark, E. A. Ledbetter, J. A. (1986) Activation of human B cells mediated through two distinct cell surface differentiation antigens, Bp35 and Bp50. Proc. Natl. Acad. Sci.:USA. 83, 4494-4498.
3. Lederman, S., Yellin, M. J., Krichevsky, A., Belko, J., Lee, J. J. Chess, L. (1992) Identification of a novel surface protein on activated CD4+ T cells that induces contact-dependent B cell differentiation (help). J Exp Med. 175, 1091-1101.
4. Lane, P., Traunecker, A., Hubele, S., Inui, S., Lanzavecchia, A. Gray, D. (1992) Activated human T cells express a ligand for the B cell-associated antigen CD40 which participates in T cell-dependent activation of B lymphocytes. Eur J Immunol. 22, 2573-2578.
5. Armitage, R. J., Fanslow, W. C., Strockbine, L., Sato, T. A., Clifford, K. N., Macduff, B. M., Anderson, D. M., Gimpel, S. D., Davis, S. T., Maliszewski, C. R. et, a.l. (1992) Molecular and biological characterization of a murine ligand for CD40. Nature. 357, 80-82.
6. Graf, D., Korthauer, U., Mages, H. W., Senger, G. Kroczek, R. A. (1992) Cloning of TRAP, a ligand for CD40 on human T cells. Eur J Immunol. 22, 3191-3194.
7. Hollenbaugh, D., Grosmaire, L. S., Kullas, C. D., Chalupny, N. J., Braesch-Andersen, S., Noelle, R. J., Stamenkovic, I., Ledbetter, J. A. Aruffo, A. (1992) The human T cell antigen gp39, a member of the TNF gene family, is a ligand for the CD40 receptor: experssion of a soluble form of gp39 with B cell co-stimulatory activity. EMBO J. 11, 4313-4321.
8. Noelle, R. J., Roy, M., Shepherd, D. M., Stamenkovic, I., Ledbetter J. A. Aruffo, A. (1992) A 39-kDa protein on activated helper T cells binds CD40 and transduces the signal for cognate activation of B cells. Proc Natl Acad Sci USA. 89, 6550-6554.
9. Lederman, S., Yellin, M. J., Cleary, A. M., Fortune, S. M. Chess, L. (1994) The understanding of contact-dependent T-cell helper function in molecular, cellular and physiological detail. Res Immunol. 145, 215-220.
10. Noelle, R. J., Ledbetter, J. A. Aruffo, A. (1992) CD40 and its ligand, an essential ligand-receptor pair for thymus-dependent B-cell activation. Immunol Today. 13, 431-433.
11. Banchereau, J., Bazan, F., Blanchard, D., Briere, F., Galizzi, J. P., van Kooten, C., Liu, Y. J., Rousset, F. Saeland, S. (1994) The CD40 antigen and its ligand. Annu. Rev. Immunol. 12, 881-922.
12. Korthauer, U., D. Graf, H. W. Mages, F. Briere, M. Padayachee, S. Malcolm, A. G. Ugazio, L. D. Notarangelo, R. L. Levinsky and R. A. Kroczek. 1993. Defective expression of T-cell CD40 ligand causes X-linked Immunodeficiency with hyper-IgM. Nature. 361: 539.
13. DiSanto, J. P., J. Y. Bonnefoy, J. F. Gauchat, A. Fischer and G. de Saint Basile. 1993. CD40 ligand mutations in X-linked immunodeficiency with hyper-IgM. Nature. 361: 541.
14. Allen, R. C., R. J. Armitage, M. E. Conley, H. Rosenblatt, N. A. Jenkins, N. G. Copeland, M. A. Bedell, S. Edelhoff, C. M. Disteche, D. K. Simoneaux and a. l. et. 1993. CD40 ligand gene defects responsible for X-linked hyper-IgM syndrome. Science. 259: 990.
15. Aruffo, A., M. Farrington, D. Hollenbaugh, X. Li, A. Milatovich, S. Nonoyama, J. Bajorath, L. S. Grosmaire, R. Stenkamp, M. Neubauer and a. l. et. 1993. The CD40 ligand, gp39, is defective in-activated T cells from patients with X-linked hyper-IgM syndrome. Cell. 72: 291.
16. Ramesh, N., R. Fuleihan, V. Ramesh, S. Lederman, H. J. Yellin, S. Sharma, L. Chess, F. S. Rosen and R. S. Geha. 1993. Deletions in the ligand for CD40 in X-linked immunoglobulin deficiency with normal or elevated IgM (HIGMX-1). Int Immunol. 5: 769.
17. Kawabe, T., T. Naka, K. Yoshida, T. Tanaka, H. Fujiwara, S. Suematsu, N. Yoshida, T. Kishimoto and H. Kikutani. 1994. The immune response in CD40-deficient mice: impaired immunoglobulin class switching and germinal center formation. Immunity. 1: 167.
18,. Xu, J., T. M. Foy, J. D. Laman, E. A. Elliot, J. J. Dunn, T. J. Waldschmidt, J. Elsemore, R. J. Noelle and R. A. Flavell. 1994. Mice deficient for the CD40 ligand. Immunity. 1:423.
19. Alderson, M. R., R. J. Armitage, T. W. Tough, L. Strockbine, W. C. Fanslow and M. K. Spriggs. 1993. CD40 expression by human monocytes: regulation by cytokines and activation of monocytes by the ligand for CD40. J Exp Mod. 178: 669.
20. Caux, C., C. Massacrier, B. Banbervliet, B. Dubois, C. Van Kooten, I. Durand and J. Banchereau. 1994. Activation of human dendritic cells through CD40 cross-linking. J. Exp. Med. 180: 1263.
21. Galy, A. H. and H. Spits. 1992. CD40 is functionally expressed on human thymic epithelial cells. J Immunol. 149: 775.
22. Freudenthal, P. S. Steinman, R. M. (1990) The distinct surface of human blood dendritic cells, as observed after an improved isolation method. Proc. Natl. Acad. Sci. USA. 87, 7698-7702.
23. Young, L. S., Dawson, C. W., Brown, K. W. Rickinson, A. B. (1989) Identification of a human epithelial cell surface protein sharing an epitope with the C3d/Epstein-Barr virus receptor of B lymphocytes. Int. J. Cancer. 43, 786-794.
24. Valent, P., Majdic, O., Maurer, D., Bodger, M., Muhm, M. Bettelheim, P. (1990) Further characterization of surface membrane structures expressed on human basophils and mast cells. Int Arch Allergy Appl Immunol. 91, 198-203.
25. O'Grady, J. T., Stewart, S., Lowrey, J., Howie, S. E. M. Krajewski, A. S. (1994) CD40 expression in hodgkin's disease. Am. J. Path. 144, 21-26.
26. Potocnik, A. J., Kinne, R., Menninger, H., Zacher, J., Emmrich, F. Kroczek, R. A. (1990) Expression of activation antigens on T cells in rheumatoid arthritis patients. Scand. J. Immunol. 31, 213-224.
27. Bevilacqua, M. P. 1993. Endothelial-leukocyte adhesion molecules. Ann. Rev. Immun l. 11: 767.
28. Springer, T. A. 1994. Traffic signals for lymphocyte recirculation and leukocyte emigration: the multistep paradigm. Cell. 76: 301.
29. Bevilacqua, M. P., S. Stengelin, M. A. Gimbrone Jr. and B. Seed. 1989. Endothelial leukocyte adhesion molecule 1: an inducible receptor for neutrophils related to complement regulatory proteins and lectins. Science. 44: 1160.
30. Graber, N., T. Venkat Gopal, D. Wilson, L. Dawson Beall, T. Polte and W. Newman. 1990. T cells bind to cytokine-activated endothelial cells via a novel, inducible sialoglycoprotein and endothelial leukocyte adhesion molecule-1. J. Immunol. 145: 819.
31. Elices, M. J., L. Osborn, Y. Takada, C. Crouse, S. Luhowsky, M. E. Hemler and R. R. Lobb. 1990. VCAM-1 on activated endothelium interacts with the leukocyte integrin VLA-4 at a site distinct from the VLA-4/fibronectin binding site. Cell. 60: 577.
32. Picker, L. J., T. K. Kishimoto, C. Wayne Smith, R. Aaron Warnock and E. C. Butcher. 1991. ELAM-1 is an adhesion molecule for skin-homing T cells. Nature. 349: 796.
33. Shimizu, Y., S. Shaw, N. Graber, T. Venkat Gopal, K. J. Horgan, G. A. Van Seventer and W. Newman. 1991. Activation-independent binding of human memory T cells to adhesion molecule ELAM-1. Nature. 349: 799.
34. Weller, P. F., T. H. Rand, S. E. Goelz, G. Chi-Rosso and R. R. Lobb. 1991. Human eosinophil adherence to vascular endothelium mediated by binding to vascular cell adhesion molecule 1 and endothelial leukocyte adhesion molecule 1. Proc. Nat. Acad. Sci, USA. 88: 7430.
35. Weller, A., S. Isenmann and D. Vestweber. 1992. Cloning of the mouse endothelial selecting. Expression of both E- and P-selectin is, inducible by tumor necrosis factor α. J. Biol. Chem. 267: 15176.
36. Pober, J. S. and R. S. Cotran. 1991. Immunologic interactions of T lymphocytes with vascular endothelium. Adv Immunol. 50: 261.

References for First Series of Experiments
1. Pauli, S., Ehlin-Henriksson, B., Mellstedt, H., Koho, H., Ben-Aissa, H. Perlmann, P. (1985) A p50 surface antigen restricted to human urinary bladder carcinomas and B lymphocytes. Cancer Immunol. Immunother. 20, 23-28.
2. Clark, E. A. Ledbetter, J. A. (1986) Activation of human B cells mediated through two distinct cell surface differentiation antigens, Bp35 and Bp50. Proc. Natl. Acad. Sci. USA. 83, 4494-4498.
3. Lederman, S., Yehlin, M. J., Krichevsky, A., Belko, J., Lee, J. J. Chess, L. (1992) Identification of a novel surface protein on activated CD4+ T cells that induces contact-dependent B cell differentiation (help). J Exp Med. 175, 1091-1101.
4. Lane, P., Traunecker, A., Hubele, S., Inui, S., Lanzavecchia, A. Gray, D. (1992) Activated human T cells express a ligand for the B cell-associated antigen CD40 which participates in T cell-dependent activation of B lymphocytes. Eur J Immunol. 22, 2573-2578.
5. Armitage, R. J., Fanslow, W. C., Strockbine, L., Sato,
T. A., Clifford, K. N., Macduff, B. M., Anderson, D. M., Gimpel, S. D., Davis, S. T., Maliszewski, C. R. et, a.l. (1992) Molecular and biological characterization of a murine ligand for CD40. Nature. 357, 80-82.
6. Graf, D., Korthauer, U., Mages, H. W., Senger, G. Kroczek, R. A. (1992) Cloning of TRAP, a ligand for CD40 on human T cells. Eur J Immunol. 22, 3191-3194.
7. Hollenbaugh, D., Grosmaire, L. S., Kullas, C. D., Chalupny, N. J., Braesch-Andersen, S., Noelle, R. J., Stamenkovic, I., Ledbetter, J. A. Aruffo, A. (1992) The human T cell antigen gp39, a member of the TNF gene family, is a ligand for the CD40 receptor: experssion of a soluble form of gp39 with B cell co-stimulatory activity. EMBO J. 11, 4313-4321.
8. Noelle, R. J., Roy, M., Shepherd, D. M., Stamenkovic, I., Ledbetter, J. A. Aruffo, A. (1992) A 39-kDa protein on activated helper T cells binds CD40 and transduces the signal for cognate activation of B cells. Proc Natl Acad Sci USA. 89, 6550-6554.
9. Lederman, S. , Yellin, M. J., Cleary, A. M., Fortune, S. M. Chess, L. (1994) The understanding of contact-dependent T-cell helper function in molecular, cellular and physiological detail. Res Immunol. 145, 215-220.
10. Noelle, R. J., Ledbetter, J. A. Aruffo, A. (1992) CD40 and its ligand, an essential ligand-receptor pair for thymus-dependent B-cell activation. Immunol Today. 13, 431-433.
11. Banchereau, J., Bazan, F., Blanchard, D., Briere, F., Galizzi, J. P., van Kooten, C., Liu, Y. J., Rousset, F. Saeland, S. (1994) The CD40 antigen and its ligand. Annu. Rev. Immunol. 12, 881-922.
12. Alderson, M. R., Armitage, R. J., Tough, T. W., Strockbine, L., Fanslow, W. C. Spriggs, M. K. (1993) CD40 expression by human monocytes: regulation by cytokines and activation of monocytes by the ligand for CD40. J Exp Med. 178, 669-674.
13. Freudenthal, P. S. Steinman, R. M. (1990) The distinct surface of human blood dendritic cells, as observed after an improved isolation method. Proc. Natl. Acad. Sci. USA. 87, 7698-7702.
14. Young, L. S., Dawson, C. W., Brown, K. W. Rickinson, A. B. (1989) Identification of a human epithelial cell surface protein sharing an epitope with the C3d/Epstein-Barr virus receptor of B lymphocytes. Int. J. Cancer. 43, 786-794.
15. Galy, A. H. Spits, H. (1992) CD40 is functionally expressed on human thymic epithelial cells. J Immunol. 149, 775-782.
16. Valent, P., Majdic, O., Maurer, D., Bodger, M., Muhm, M. Bettelheim, P. (1990) Further characterization of surface membrane structures expressed on human basophils and mast cells. Int Arch Allergy Appl Immunol. 91,198-203.
17. O'Grady, J. T., Stewart, S., Lowrey, J., Howie, S. E. M. Krajewski, A. S. (1994) CD40 expression in hodgkin's disease. Am. J. Path. 144, 21-26.
18. Potocnik, A. J., Kinne, R., Menninger, H., Zacher, J., Emmrich, F. Kroczek, R. A. (1990) Expression of activation antigens on T cells in rheumatoid arthritis patients. Scand. J. Immunol. 31, 213-224.
19. Arnett, F. C., Edworthy, S. M., Bloch, D. A., McShane, D. J., Fries, J. F., Cooper, N. S., Healey, L. A., Kapkan, S. R., Liang, M. H., Luthra, H. S., Medsger, T. A. J., Mitchell, D. M., Neustadt, D. H., Pinals, R. S., Schaller, J. G., Sharp, J. T., Wilder, R. L. Hunder, G. G. (1988) The American Rheumatism Association 1987 revised criteria for the classification of rheumatoid arthritis. Arthritis Rheum. 31, 315-324.
20. Yellin, M. J., Sinning, J., Covey, L. R., Sherman, W., Lee, J. J., Glickman, N. E., Sippel, K. C., Rogers, J., Cleary, A. M., Parker, M. et, a.l. (1994) T lymphocyte T cell-B cell-activating molecule/CD40-L molecules induce normal B cells or chronic lymphocytic leukemia B cells to express CD80 (B7/BB-1) and enhance their costimulatory activity. J Immunol. 153, 666-674.
21. Yellin, M. J., Lee, J. J., Chess, L. Lederman, S. (1991) A human CD4-T cell leukemia subclone with contact-dependent helper function. J Immunol. 147, 3389-3395.
22. Aarden, L. A., De Groot, E. R., Schaap, O. L. Lansdorp, P. M. (1987) Production of hybridoma growth factor by human monocytes. Eur. J. Immunol. 17, 1411-1416.
23. Stamenkovic, I., Clark, E. A. Seed, B. (1989) A B-lymphocyte activation molecule related to the nerve growth factor receptor and induced by cytokines in carcinomas. EMBO J. 8, 1403-1410.
24. Springer, T. A. (1990) Adhesion receptors of the immune system. Nature. 346, 425-434.
25. Valle, A., Zuber, C. E., Defrance, T., Djossou, O., De Rie, M. Banchereau, J. (1989) Activation of human B lymphocytes through CD40 and interleukin 4. Eur. J. Immunol. 19, 1463-1467.
26. Ranheim, E. A. Kipps, T. J. (1993) Activated T cells induce expression of B7/BB1 on normal or leukemic B cells through a CD40-dependent signal. J Exp Med. 177, 925-935.
27. Raynaud,. F., Bauvois, B., Gerbaud, P. Evain, B. D. (1992) Characterization of specific proteases associated with the surface of human skin fibroblasts, and their modulation in pathology. J Cell Physiol. 151, 378-385.
28. Clark, E. A. Shu, G. (1990) Association between IL-6 and CD40 signalling: IL-6 induces phosphorylation of CD40 receptors. J Immunol. 145, 1400-1406.
29. Firestein, G. S., Alvaro, G. J. M., Maki, R. Alvaro, G. J. M. (1990) Quantitative analysis of cytokine gene expression in rheumatoid arthritis. J Immunol. 144, 3347-3353.
30. Arend, W. P. Dayer, J. M. (1990) Cytokines and cytokine inhibitors or antagonists in rheumatoid arthritis. Arthritis Rheum. 33, 305-315.
31. Bucala, R., Ritchlin, C., Winchester, R. Cerami, A. (1991) Constitutive production of inflammatory and mitogenic cytokines by rheumatoid synovial fibroblasts. J Exp Med. 173, 569-574.
32. Butler, D. M., Piccoli, D. S., Hart, P. H. Hamilton, J. A. (1988) Stimulation of human synovial fibroblast DNA synthesis by recombinant human cytokines. J Rheumatol. 15, 1463-1470.
33. Fleischmajer, R., Perlish, J. S. Reeves, J. R. T. (1977) Cellular Infiltrates in scleroderma skin. Arthritis Rheum. 20, 975-983.
34. Furst, D. E., Clements, P. J., Granz , P., Gale, R. Roberts, N. (1979) A syndrome resembling progressive systemic sclerosis after bone marrow transplantation. A model for scleroderma? Arthritis Rheum. 22, 904-910.
35. Ferrara, J. L. M. Deeg, H. J. (1991) Mechanisms of disease. Graft-versus-host disease. N. Engl. J. Med. 324, 667-674.
36. Fishel, R., Barbul, A., Wasserkrug, H. L., Penberthy, L. T., Rettura, G. Efron, G. (1983) Cyclosporine A impairs wound healing in rats. J. Surg. Res. 34, 572-575.
37. Peterson, J. M., Barbul, A., Breslin, R. J., Wasserkrug, H. L. Efron, G. (1987) Significance of T-lymphocytes in wound healing. Surgery. 102, 300-304.
38. Schrier, D. J., Phan, S. H. McGarry, B. M. (1983) The effects of the nude (nu/nu) mutation on bleomycin-induced pulmonary fibrosis. Am. Rev. Respir. Dis. 127, 614-617.
39. Allen, J. B., Malone, D. G., Wahl, S. M., Calandra, G. B. Wilder, R. L. (1985) Role of the thymus in streptococcal cell wall-induced arthritis and hepatic granuloma formation. Comparative studies of pathology and cell wall distribution in athymic and euthymic rats. J. Clin. Invest. 76, 1042-1056.
40. Wahl, S. M., Hunt, D. A., Allen, J. B., Wilder, R. L., Paglia, L. Hand, A. R. (1986) Bacterial cell wall-induced hepatic granulomas. An in vivo model of T cell-dependent fibrosis. J. Exp. Med. 163, 884-902.
41. Dayer, J. M., Breard, J., Chess, L. Krane, S. M. (1979) Participation of monocyte-macrophages and lymphocytes in, the production of a factor that stimulates collagenase and prostaglandin release by rheumatoid synovial c lis. J. Clin. Invest. 64, 1386-1392.
42. Dayer, J. M., Beutler, B. Ceram, A. (1985) Cachectin/tumor necrosis factor stimulates collagenase and prostaglandin E2 production by human synovial cells and dermal fibroblasts. J. Exp. Med. 162, 2163-2168.
43. Dayer, J. M., de Rochemonteix, B., Burrus, B., Cemczuk, S. Dinarello, C. A. (1986) Human recombinant inteleukin 1 stimnulates collagenase and prostaglandin E2 production by human synovial cells. J Clin Invest. 77, 645-648.
44. Ishikawa, H. Ziff, M. (1976) Electron microscopic observations of immunoreactive cells in the rheumatoid synovial membrane. Arthritis Rheum. 19, 1-14.
45. Golay, J., Cusmano, G. Introna, M. (1992) Independent regulation of m-myc, B-myb, and c-myb gene expression by inducers and inhibitors of proliferation in human B lymphocytes. J Immunol. 149, 300-308.
46. Qu, Z., Hernandez Garcia, C., O'Rourke, L. M., Planck, S. R., Kohli, M. Rosenbaum, J. T. (1994) Local proliferation of fibroblast-like synoviocytes contributes to synovial hyperplasia: results of proliferating cel nuclear antigen/cyclin, c-myc, and nucleolar organizer staining. Arthritis Rheum. 2, 212-220.
47. Van Seventer, G. A., Newman, W., Shimizu, Y., Nutman, T. B., Tanaka, Y., Horgan, K. J., Gopal, T. V., Ennis, E., O'Sullivan, D., Grey, H. Shaw, S. (1991) Analysis of T cell stimulation by superantigen plus major histocompatibility complex class II molecules or by CD3 monoclonal antibody: costimulation by purified adhesion ligands VCAM-1, ICAM-1, but not ELAM-1. J Exp Med. 174, 901-913.
48. Hale, L. P., Martin, M. E., McCollum, D. E., Nunley, J. A., Springer, T. A., Sing r, K. H. Haynes, B. F. (1989) Immunohistologic analysis of the distribution of cell adhesion molecules within the inflammatory synovial microenvironment. Arthritis Rheum. 32, 22-30.
49. Morales, D. J., Wayner, E., Elices, M. J., Alvaro, G. J. M., Zvaifler, N. J. Firestein, G. S. (1992) Alpha 4/beta 1 integrin (VLA-4) ligands in arthritis. Vascular cell adhesion molecule-1 expression in synovium and on fibroblast-like synoviocytes. J Immunol. 149, 1424-1431.
-50. Kriegsmann, J., Keyszer, G. M., Geiler, T., Brauer, R., Gay, R. E. Gay, S. (1995) Expression of vascular cell adhesion molecule-1 mRNA and protein in rheumatoid synovium demonstrated by in situ hybridization and immunohistochemistry. Lab Invest. 72, 208-214.
51. Marlor, C. W., Webb, D. L., Bombara, M. P., Greve, J. M. Blue, M. L. (1992) Expression of vascular cell adhesion molecule-1 in fibroblastlike synoviocytes after stimulation with tumor necrosis factor. Am J Pathol. 140, 1055-1060.
52. Chin, J. E., Winterrowd, G. E., Krzesicki, R. F. Sanders, M. E. (1990) Role of cytokines in inflammatory synovitis. The coordinate regulation of intercellular adhesion molecule 1 and HLA class I and class II antigens in rheumatoid synovial fibroblasts. Arthritis Rheum. 33, 1776-1786.
53. Krzesicki, R. F., Fleming, W. E., Winterrowd, G. E., Hatfield, C. A., Sanders, M. E. Chin, J. E. (1991) T lymphocyte adhesion to human synovial fibroblasts. Role of cytokines and the interaction between intercellular. adhesion molecule 1 and CD11a/CD18. Arthritis Rheum. 34, 1245-1253.
54. Durie, F. H., Fava, R. A., Foy, T. M., Aruffo, A., Ledbetter, J. A. Noelle, R. J. (1993) Prevention of collagen-induced arthritis with an antibody to gp39, the ligand for CD40. Science. 261, 1328-1330.

References for Second Series of Experiments
1. BAevilacqua, M. P. 1993. Endothelial-leukocyte adhesion molecules. Ann. Rev. Immunol. 11: 767.
2. Springer, T. A. 1994. Traffic signals for lymphocyte recirculation and leukocyte emigration: the multistep paradigm. Cell. 76: 301.
3. Bevilacqua, M. P., S. Stengelin, M. A. Gimbrone Jr. and B. Seed. 1989. Endothelial leukocyte adhesion molecule 1: an inducible receptor for neutrophils related to complement regulatory proteins and lectins. Science. 44: 1160.
4. Graber, N., T. Venkat Gopal, D. Wilson, L. Dawson Beall, T. Polte and W. Newman. 1990. T cells bind to cytokine-activated endothelial cells via a novel, inducible sialoglycoprotein and endothelial leukocyte adhesion molecule-1. J. Immunol. 145: 819.
5. Elices, M. J., L. Osborn, Y. Takada, C. Crouse, S. Luhowsky, M. E. Hemler and R. R. Lobb. 1990. VCAM-1 on activated endothelium interacts with the leukocyte integrin VLA-4 at a site distinct from the VLA-4/fibronectin binding site. Cell. 60: 577.
6. Picker, L. J., T. K. Kishimoto, C. Wayne Smith, R. Aaron Warnock and E. C. Butcher. 1991. ELAM-1 is an adhesion molecule for skin-homing T cells. Nature. 349: 796.
7. Shimizu, Y., S. Shaw, N. Graber, T. Venkat Gopal, K. J. Horgan, G. A. Van Seventer and W. Newman. 1991. Activation-independent binding of human memory T cells to adhesion molecule ELAM-1. Nature. 349: 799.
8. Weller, P. F., T. H. Rand, S. E. Goelz, G. Chi-Rosso and R. R. Lobb. 1991. Human eosinophil adherence to vascular endothelium mediated by binding to vascular cell adhesion molecule 1 and endothelial leukocyte adhesion molecule 1. Proc. Nat. Aced. Sci, USA. 88: 7430.
9. Weller, A., S. Isenmann and D. Vestweber. 1992. Cloning of the mouse endothelial selecting. Expression of both E- and P-selectin is inducible by tumor necrosis factor α. J. Biol. Chem. 267: 15176.
10. Pober, J. S. and R. S. Cotran. 1991. Immunologic interactions of T lymphocytes with vascular endothelium. Adv Imunol. 50: 261.
11. Potocnik, A. J., R. Kinne, H. Menninger, J. Zacher, F. Emmrich and R. A. Kroczek. 1990. Expression of activation antigens on T cells in rheumatoid arthritis patients. Scand. J. Immunol. 31: 213.
12. Pauli, S., B. Ehlin-Henriksson, H. Mellstedt, H. Koho, H. Ben-Aissa and P. Perlmann. 1985.A p50 surface antigen restricted to human urinary bladder carcinomas and B lymphocytes. Cancer Immunol. Immunother. 20: 23.
13. Clark, E. A. and J. A. Ledbetter. 1986. Activation of human B cells mediated through two distinct cell surface differentiation antigens, Bp35 and Bp50. Proc. Natl. Acad. Sci. USA. 83: 4494.
14. Alderson, M. R. , R. J. Armitage, T. W. Tough, L. Strockbine, W. C. Fanslow and M. K. Spriggs. 1993. CD40 expression by human monocytes: regulation by cytokines and activation of monocytes by the ligand for CD40. J Exp Med. 178: 669.
15. Freudenthal, P. S. and R. M. Steinman. 1990. The distinct surface of human blood dendritic cells, as observed after an improved isolation method. Proc. Natl. Acad. Sci. USA. 87: 7698.
16. Valent, P., O. Majdic, D. Maurer, M. Bodger, M. Muhm and P. Bettelheim. 1990. Further characterization of surface membrane structures expressed on human basophils and mast cells. Int Arch Allergy Appl Immunol. 91: 198.
17. Young,. L. S., C. W. Dawson, K. W. Brown and A. B. Rickinson. 1989. Identification of a human epithelial cell surface protein sharing an epitope with the C3d/Epstein-Barr virus receptor of B lymphocytes. Int. J. Cancer. 43: 786.
18. Galy, A. H. and H. Spits. 1992. CD40 is functionally expressed on human thymic epithelial cells. J Immunol. 149: 775.
20. Lederman, S., M. J. Yellin, A. Krichevsky, J. Belko, J. J. Lee and L. Chess. 1992. Identification of a novel surface protein on activated CD4+ T cells that induces contact-dependent B cell differentiation (help). J Exp Med. 175: 1091.
21. Lane, P., A. Traunecker, S. Hubele, S. Inui, A. Lanzavecchia and D. Gray. 1992. Activated human T cells express a ligand for the B cell-associated antigen CD40 which participates in T cell-dependent activation of B lymphocytes. Eur J Immunol. 22: 2573.
22. Armitage, R. J., W. C. Fanslow, L. Strockbine, T. A. Sato, K. N. Clifford, B. M. Macduff, D. M. Anderson, S. D. Gimpel, S. T. Davis, C. R. Maliszewski and a. .et. 1992. Molecular and biological characterization of a murine ligand for CD40. Nature. 357: 80.
23. Graf, D., U. Korthauer, H. W. Mages, G. Senger and R. A. Kroczek. 1992. Cloning of TRAP, a ligand for CD40 on human T cells. Eur J Immunol. 22: 3191.
24. Hollenbaugh, D., L. S. Grosmaire, C. D. Kullas, N. J. Chalupny, S. Braesch-Andersen, R. J. Noelle, I. Stamenkovic, J. A. Ledbetter and A. Aruffo. 1992. The human T cell antigen gp39, a member of the TNF gene family, is a ligand for the CD40 receptor: experssion of a soluble form of gp39 with B cell co-stimulatory activity. EMBO J. 11: 4313.
25. Noelle, R. J., M. Roy, D. M. Shepherd, I. Stamenkovic, J. A. Ledbetter and A. Aruffo. 1992. A 39-kDa protein on activated helper T cells binds CD40 and transduces the signal for cognate activation of B cells. Proc Natl Acad Sci USA. 89: 6550.
26. Lederman, S., M. J., Yellin, A. M. Cleary, S. M. Fortune and L. Chess. 1994. The understanding of contact-dependent T-cell helper function in molecular, cellular and physiological detail. Res Immunol. 145: 215.
27. Noelle, R. J., J. A. Ledbetter and A. Aruffo. 1992. CD40 and its ligand, an essential ligand-receptor. pair for thymus-dependent B-cell activation. Immunol Today. 13: 431.
28. Banchereau, J., F. Bazan, D. Blanchard, F. Briere, J. P. Galizzi, C. van Kooten, Y. J. Liu, F. Rousset and S. Saeland. 1994. The CD40 antigen and its ligand. Annu. Rev. Immunol . 12: 881.
29. Korthauer, U., D. Graf, H. W. Mages, F. Briere, M. Padayachee, S. Malcolm, A. G. Ugazio, L. D. Notarangelo, R. L. Levinsky and R. A. Kroczek. 1993. Defective expression of T-cell CD40 ligand causes X-linked Immunodeficiency with hyper-IgM. Nature. 361: 539.
30. DiSanto, J. P., J. Y. Bonnefoy, J. F. Gauchat, A. Fischer and G. de Saint Basile. 1993. CD40 ligand mutations in X-linked immunodeficiency with hyper-IgM. Nature. 361: 541.
31. Allen, R. C., R. J. Armitage, M. E. Conley, H. Rosenblatt, N. A. Jenkins, N. G. Copeland, M. A. Bedell, S. Edelhoff, C. M. Disteche, D. K. Simoneaux and a. l. et. 1993. CD40 ligand gene defects responsible for X-linked hyper-IgM syndrome. Science. 259: 990.
32. Aruffo, A., M. Farrington, D. Hollenbaugh, X. Li, A. Milatovich, S. Nonoyama, J. Bajorath, L. S. Grosmaire, R. Stenkamp, M. Neubauer and a. l. et. 1993. The CD40 ligand, gp39, is defective in activated T cells from patients with X-linked hyper-IgM syndrome. Cell. 72: 291.
33. Ramesh, N., R. Fuleihan, V. Ramesh, S. Lederman, M. J. Yellin, S. Sharma, L. Chess, F. S. Rosen and R. S. Geha. 1993. Deletions in the ligand for CD40 in X-linked immunoglobulin deficiency with normal or elevated IgM (HIGMX-1). Int Immunol. 5: 769.
34. Kawabe, T., T. Naka, K. Yoshida, T. Tanaka, H. Fujiwara, S. Suematsu, N. Yoshida, T. Kishimoto and H. Kikutani. 1994. The immune response in CD40-deficient mice: impaired immunoglobulin class switching and germinal center formation. Immunity. 1: 167.
35. Xu, J., T. M. Foy, J. D. Laman, E. A. Elliot, J. J. Dunn, T. J. Waldschmidt, J. Elsemore, R. J. Noelle and R. A. Flavell. 1994. Mice deficient for the CD40 ligand. Immunity. 1: 423.
36. Caux, C., C. Massacrier, B. Banbervliet, B. Dubois, C. Van Kooten, I. Durand and J. Banchereau. 1994. Activation of human dendritic cells through CD40 cross-linking. J. Exp. Med. 180: 1263.
37. Yellin, M. J., J. Sinning, L. R. Covey, W. Sherman, J. J. Lee, N. E. Glickman, K. C. Sippel, J. Rogers, A. M. Cleary,. M. Parker and a. l. et. 1994. T lymphocyte T cell-B cell-activating molecule/CD40-L molecules induce normal B cells or chronic lymphocytic leukemia B cells to express CD80 (B7/BB-1) and enhance their costimulatory activity. J Immunol. 153: 666.
38. Lederman, S., M. J. Yellin, G. Inghirami, J.;J. Lee, D. M. Knowles and L. Chess. 1992. Molecular interactions mediating T-B lymphocyte collaboration in human lymphoid follicles. Roles of T cell-B-cell-activating molecule (5c8 antigen) and CD40 in contact-dependent help. J Immunol. 149: 3817.
39. Lederman, S., M. J. Yellin, A. M. Cleary, A. Pernis, G. Inghirami, L. E. Cohn, L. R. Covey, J. J. Lee, P. Rothman and L. Chess. 1994. T-BAM/CD40-L on helper T lymphocytes augments lymphokine-induced B cell Ig isotype switch recombination and rescues B cells from programmed cell death. J Immunol. 152: 2163.
40. Jaffe, E., R. Nachman, C. Becker and R. Minick. 1973. Culture of human endothelial cells derived from umbilical veins. Identification by morphologic and immunologic criteria. J. Clin. Invest. 52: 2745.
41. Thornton, S., S. Mueller and E. Levine. 1983. Human endothelial cells: use of heparin in long term cloning and serial cultivation. Science. 222: 623.
42. Yellin, M. J., J. J. Lee, L. Chess and S. Lederman. 1991. A human CD4− T cell leukemia subclone with contact-dependent helper function. J Immunol. 147: 3389.
43. Holthofer, H., I. Virtanen, A. L. Kariniemi, M. Hormia, E. Linder and A. Miettinen. 1982. Ulex europaeus I lectin as a marker for vascular endothelium in human tissue. Lab. Invest. 47: 60.
44. Fina, L., H. V. Molgaard, D. Robertson, N. J. Bradley, P. Monaghan, D. Delia, R. D. Sutherland, M. A. Baker and M. F. Greaves. 1990. Expression of the CD34 gene in vascular endothelial cells. Blood. 75: 2417.
45. Stamenkovic, I., E. A. Clark and B. Seed. 1989. A B-lymphocyte activation molecule related to the nerve growth factor receptor and induced by cytokines in carcinomas. EMBO J. 8: 1403.
46. Ranheim, E. A. and T. J. Kipps. 1993. Activated T cells induce expression of B7/BB1 on normal or leukemic B cells through a CD40-dependent signal. J Exp Med. 177: 925.
47. Hughes, C. C., C. O. Savage and J. S. Pober. 1990. The, endothelial cell as a regulator of T-cell function. Immunol Rev. 117: 85.
48. Hughes, C. C. W., C. O. S. Savage and J. S. Prober. 1990. Endothelial cells augment T cell interleukin 2 production by a contact-dependent mechanism involving CD2/LFA-3 interactions. J. Exp. Med. 171: 1453.
49. Guinan, E. C., B. R. Smith, J. T. Doukas, R. A. Miller and J. S. Pober. 1989. Vascular endothelial cells enhance T cell responses by markedly augmenting IL-2 concentrations. Cell. Immunol. 118: 166.
50. Azuma, M., D. Ito, H. Yagita, K. Okumura, J. H. Phillips, L. L. Lanier and C. Somoza. 1993. B70 antigen is a second ligand for CTLA-4 and CD28. Nature. 366: 76.
51. Kennedy, M. K., K. M. Mohler, K. D. Shanebeck, P. R., Baum, K. S. Picha, C. A. Otten-Evans, C. A. Janeway and K. H. Grabstein. 1994. Induction of B cell costimulatory function by recombinant murine CD40 ligand. Eur. J. Immunol. 24: 116.
52. Maliszewski, C. R., K. Grabstein, W. C. Fanslow, R. Armitage, M. K. Spriggs and T. A. Sato. 1993. Recombinant CD40 ligand stimulation of murine B cell growth and differentiation: cooperative effects of cytokines. Eur J Immunol. 23: 1044.
53. Spriggs, M. K., R. J. Armitage, L. Strockbine, K. N. Clifford, B. M. Macduff, T. A. Sato, C. R. Maliszewski and. W. C. Fanslow. 1992. Recombinant human CD40 ligand stimulates B cell proliferation and immunoglobulin E secretion. J Exp Med. 176: 1543.
54. Yellin, M. J., K. Sippel, G. Inghirami, L. R. Covey, J. J. Lee, J. Sinning, E. A. Clark, L. Chess and S. Lederman. 1994. CD40 molecules induce down-modulation and endocytosis of T cell surface T cell-B cell activating molecule/CD40-L. Potential role in regulating helper effector function. J Immunol. 152: 598.
55. Barrett, T. B., G. Shu and E. A. Clark. 1991. CD40 signalling activates, CD11a/CD18 (LFA-1)-mediated adhesion in B cells. J. Immunol. 146: 1722.
56. Flores-Romo, L., D. Estoppey and K. B. Bacon. 1993. Anti-CD40 antibody stimulates the VLA-4-dependent adhesion of normal and LFA-1-deficient B cells to endothelium. Immunology. 79: 445.
57. Collins, T., A. J. Korman, C. T. Wake, J. M. Boss, D. J. Kappes, W. Fiers, K. A. Ault, M. A. Gimbrone Jr., J. L. Strominger and J. S. Prober. 1984. Immune interferon activates multiple class II major histocompatibility complex genes and the associated invariant chain gene in human endothelial cells and dermal fibroblasts. Proc. Natl. Acad. Sci, USA. 81: 4917.
58. Barkley, D., S. Allard, M. Feldmann and R. N. Maini. 1989. Increased expression of HLA-DQ antigens by interstitial cells and endothelium in the synovial membrane of rheumatoid arthritis patients compared with reactive arthritis patients. Arthrit. Rheum. 32: 955.
59. Gruschwitz, M., N. Sepp, H. Kofler and G. Wick. 1991. Expression of class II-MHC antigens in the dermis of patients with progressive systemic sclerosis. Immunobiology., 182: 234.
60. Salomon, R. N., C. C. W. Huges, F. J. Schoen, D. D. Payne, J. S. Pober and P. Libby. 1991. Human coronary transplantation-associated arteriosclerosis. Evidence for a chronic immune reaction to activated graft endothelial cells. Am. J. Path. 138: 791.
61. Murray, A. G., M. M. Khodadoust, J. S. Pober and A. L. M. Bothwell. 1994. Porcine aortic endothelial cells activate human T cells: direct presentation of MHC antigens and costimulation by ligands for human CD2 and CD28. Immunity. 1: 57.
62. Koch, A. E., J. C. Burrows, G. K. Haines, T. M. Carlos, J. M. Harlan and S. Joseph Leibovich. 1991. Immunolocalization of endothelial and leukocyte adhesion molecules in human rheumatoid arthritis and osteoarthritis synovial tissues. Lab. Invest. 64: 313.
63. Gruschwitz, M. S., O. P. Hornstein and P. von den Driesch. 1995. Correlation of soluble adhesion molecules in the peripheral blood of scleroderma patients with their in situ expression and with disease activity. Arthrit. Rheum. 38: 184.
64. Brockmeyer, C., M. Ulbrecht, D. J. Schendel, E. H. Weiss, G. Hillebrand, R. Burkhardt, W. Land, M. J. Gokel, G. Riethmuller and H. E. Feucht. 1993. Distribution of cell adhesion molecules (ICAM-1, VCAM-1 and ELAM-1) in renal tissue during allograft rejection. Transplantation. 55: 610.
65. Wick, G., G. Schett, A. Amberger, R. Kleindienst and Q. Xu. 1995. Is atherosclerosis an immunologically mediated disease. Immunol. Today. 16: 27.
66. Durie, F. H., R. A. Fava, T. M. Foy, A. Aruffo, J. A. Ledbetter and R. J. Noelle. 1993. Prevention of collagen-induced arthritis with an antibody to gp39, the ligand for CD40. Science. 261: 1328.
67. Durie, F. H., T. M. Foy and R. J. Noelle. 1994. The role of CD40 and its ligand (gp39) in peripheral and central tolerance and its contribution to autoimmune disease. Res. Immunol. 145: 200.

1. A method of inhibiting activation by CD40 ligand of cells bearing CD40 on the cell surface, other than B cells, comprising contacting the cells with an agent capable of inhibiting interaction between CD40 ligand and the cells, in an amount effective to inhibit activation of the cells.

2. The method of claim 1, wherein the CD40-bearing cells are selected from the group consisting of fibroblasts, endothelial cells, epithelial cells, T cells, basophils, macrophages, Reed-Steinberg cells, and dendritic cells.

3. The method of claim 2, wherein the epithelial cells are keratinocytes.

4. The method of claim 1, wherein the agent inhibits binding of CD40 ligand to CD40 on the cells.

5. The method of claim 1, wherein the agent is a protein.

6. The method of claim 5, wherein the protein comprises an antibody or portion thereof.

7. The method of claim 6, wherein the antibody is a monoclonal antibody.

8. The method of claim 7, wherein the monoclonal antibody is a chimeric antibody.

9. The method of claim 7, wherein the monoclonal antibody is a humanized antibody.

10. The method of claim 7, wherein the monoclonal antibody is a primatized antibody.

11. The method of claim 6, wherein the portion of the antibody comprises a complementarity determining region or variable region of a light or heavy chain.

12. The method of claim 6, wherein the portion of the antibody comprises a complementarity determining region or a variable region.

13. The method of claim 12, wherein the portion of the antibody comprises a Fab, or a single chain antibody.

14. The method of claim 5, wherein the protein comprises soluble extracellular region of CD40 ligand, or variants thereof including conservative substituents, or portion thereof; or soluble extracellular region of CD40, or variants thereof including, conservative substituents, or portion thereof.

15. The method of claim 14, wherein the soluble extracellular region of CD40 ligand or CD40 is a monomer.

16. The method of claim 14, wherein the soluble extracellular region of CD40 is an oligomer.

17. The method of claim 14, wherein the protein comprising soluble extracellular region of CD40 or portion thereof further comprises an Fc region fused to the extracellular region of CD40 or portion thereof.

18. The method of claim 17, wherein the Fc region is capable of binding to protein A or protein G.

19. The method of claim 17, wherein the Fc region comprises IgG, IgA, IgM, IgD, or IgE, or subclasses thereof.

20. The method of claim 19, wherein:
the IgG is IgG₁, IgG₂, IgG₃, or IgG₄; or
the IgA is IgA₁ or IgA₂.

21. The method of claim 1, wherein the agent specifically binds to the antigen to which monoclonal antibody 5c8 (ATCC Accession No. HB 10916) specifically binds.

22. The method of claim 21, wherein the agent is an antibody.

23. The method of claim 22, wherein the antibody is monoclonal antibody 5c8 (ATCC Accession No. HB 10916).

24. The method of claim 1, wherein the agent is a small molecule.

25. The method of claim 1, wherein the agent specifically binds to CD40 on the cell surface.

26. The method of claim 25, wherein the agent is a protein.

27. The method of claim 26, wherein the protein is an antibody.

28. The method of claim 27, wherein the antibody is a monoclonal antibody.

29. The method of claim 28, wherein the monoclonal antibody is chimeric, humanized, or primatized.

30. The method of claim 26, wherein the protein comprises the extracellular region of CD40 ligand.

31. The method of claim 1, wherein the agent is nonprotein.

32. The method of claim 1, wherein the agent is selected from a library of known agents.

33. The method of claim 1, wherein the agent is modified from a known agent.

34. The method of claim 33, wherein the modified agent is designed by structure optimization of a lead inhibitory agent based on a three-dimensional structure of a complex of soluble extracellular region of CD40 ligand or portion thereof with the lead inhibitory agent.

35. The method of claim 1, wherein the agent is selected by a screening method, which comprises:
isolating a sample of cells;
culturing the sample under conditions permitting activation of CD40-bearing cells;
contacting the sample with cells expressing a protein which is specifically recognized by monoclonal antibody 5c8 produced by the hybridoma having ATCC Accession No. HB 10916, or with a protein which is specifically recognized by monoclonal antibody 5c8 produced by the hybridoma having ATCC Accession No. HB 10916, effective to activate the CD40-bearing cells;
contacting the sample with an amount of the agent effective to inhibit activation of the CD40-bearing cells if the agent is capable of inhibiting activation of the CD40-bearing cells; and
determining whether the cells expressing the protein which is specifically recognized by monoclonal antibody 5c8 produced by the hybridoma having ATCC Accession No. HB. 10916, or with the protein which is specifically recognized by monoclonal antibody 5c8 produced by the hybridoma having ATCC Accession No. HB 10916, activate the CD40-bearing cells in the presence of the agent.

36. The method of claim 35, wherein the agent is selected from a library of known agents.

37. The method of claim 36, wherein the known agents are nonprotein agents.

38. A method of inhibiting activation by CD40 ligand of cells bearing CD40 on the cell surface, other than B cells, in a subject, comprising administering to the subject an agent capable of inhibiting interaction between CD40 ligand and the cells, in an amount effective to inhibit activation of the cells in the subject.

39. The method of claim 38, wherein the CD40-bearing cells are selected from the group consisting of fibroblasts, endothelial cells, epithelial cells, T cells, basophils, macrophages, Reed-Steinberg cells, and dendritic cells.

40. The method of claim 39, wherein the epithelial cells are keratinocytes.

41. The method of claim 38, wherein the agent inhibits binding of CD40 ligand to CD40 on the cells.

42. The method of claim 38, wherein the agent is a protein.

43. The method of claim 42, wherein the protein comprises an antibody or portion thereof.

44. The method of claim 43, wherein the antibody is a monoclonal antibody.

45. The method of claim 43, wherein the monoclonal antibody is a chimeric antibody.

46. The method of claim 44, wherein the monoclonal antibody is a humanized antibody.

47. The method of claim 44, wherein the monoclonal antibody is a primatized antibody.

48. The method of claim 43, wherein the portion of the antibody comprises a complementarity determining region or variable region of a light or heavy chain.

49. The method of claim 43, wherein the portion of the antibody comprises a complementarity determining region or a variable region.

50. The method of claim 49, wherein the portion of the antibody comprises a Fab, or a single chain antibody.

51. The method of claim 38, wherein the agent specifically binds to the antigen to which monoclonal antibody 5c8 (ATCC Accession No. HB 10916) specifically binds.

52. The method of claim 51, wherein the agent is an antibody.

53. The method of claim 52, wherein the antibody is monoclonal antibody 5c8 (ATCC Accession No. HB 10916).

54. The method of claim 38, wherein the subject is a mammal.

55. The method of claim 54, wherein the mammalian subject is a human.

56. The method of claim 54, wherein the mammalian subject is a rodent.

57. The method of claim 38, wherein the protein comprises soluble extracellular region of CD40 ligand, or variants thereof including conservative substituents, or portion thereof; or soluble extracellular region of CD40, or variants thereof including conservative substituents, or portion thereof.

58. The method of claim 57, wherein the soluble extracellular region of CD40 ligand or CD40 is a monomer.

59. The method of claim 57, wherein the soluble extracellular region of CD40 is an oligomer.

60. The method of claim 57, wherein the protein comprising soluble extracellular region of CD40 or portion thereof further comprises an Fc region-fused to the extracellular region of CD40 or portion thereof.

61. The method of claim 60, wherein the Fc region is capable of binding to protein A or protein G.

62. The method of claim 60, wherein the Fc region comprises IgG, IgA, IgM, IgD, or IgE, or subclasses thereof.

63. The method of claim 62, wherein:
the IgG is IgG₁, IgG₂, IgG₃, or IgG₄; or
the IgA is IgA₁ or IgA₂.

64. The method of claim 38, wherein the agent is a small molecule.

65. The method of claim 38, wherein the agent specifically binds to CD40 on the cell surface.

66. The method of claim 65, wherein the agent is a protein.

67. The method of claim 66, wherein the protein is an antibody.

68. The method of claim 67, wherein the antibody is a monoclonal antibody.

69. The method of claim 68, wherein the monoclonal antibody is chimeric, humanized, or primatized.

70. The method of claim 66, wherein the protein comprises the extracellular region of CD40 ligand.

71. The method of claim 38, wherein the agent is nonprotein.

72. The method of claim 38, wherein the agent is selected from a library of known agents.

73. The method of claim 38, wherein the agent is modified from a known agent.

74. The method of claim 73 wherein the modified agent is designed by structure optimization of a lead inhibitory agent based on a three-dimensional structure of a complex of soluble extracellular region of CD40 ligand or portion thereof with the lead inhibitory agent.

75. The method of claim 38, wherein the agent is selected by a screening method, which comprises:
isolating a sample of cells;
culturing the sample under conditions permitting activation of CD40-bearing cells;
contacting the sample with cells expressing a protein which is specifically recognized by monoclonal antibody 5c8 produced by the hybridoma having ATCC Accession No. HB 10916, or with a protein which is specifically recognized by monoclonal antibody 5c8 produced by the hybridoma having ATCC Accession No. HB 10916, effective to activate the CD40-bearing cells;
contacting the sample with an amount of the agent effective to inhibit activation of the CD40-bearing cells if the agent is capable of inhibiting activation of the CD40-bearing cells; and
determining whether the cells expressing the protein which is specifically recognized by monoclonal antibody 5c8 produced by the hybridoma having ATCC Accession No. HB 10916, or with the protein which is specifically recognized by monoclonal antibody 5c8 produced by the hybridoma having ATCC Accession No. HB 10916, activate the CD40-bearing cells in the presence of the agent.

76. The method of claim 75, wherein the agent is selected from a library of known agents.

77. The method of claim 76, wherein the known agents are nonprotein agents.

78. A method of inhibiting an inflammatory response in a subject, comprising the method of claim 38.

79. A method of treating a condition dependent on CD40 ligand-induced activation of fibroblast cells in a subject, comprising the method of claim 38.

80. The method of claim 79, wherein the fibroblasts are synovial membrane fibroblasts, dermal fibroblasts, pulmonary fibroblasts, or liver fibroblasts.

81. The method of claim 79, wherein the condition is selected from the group consisting of arthritis, scleroderma, and fibrosis.

82. The method of claim 81, wherein the arthritis is rheumatoid arthritis, non-rheumatoid inflammatory arthritis, arthritis associated with Lyme disease, or osteoarthritis.

83. The method of claim 81, wherein the fibrosis is pulmonary fibrosis, hypersensitivity pulmonary fibrosis, or a pneumoconiosis.

84. The method of claim 83, wherein the pulmonary fibrosis is pulmonary fibrosis secondary to adult respiratory distress syndrome, drug-induced pulmonary fibrosis, idiopathic pulmonary fibrosis, or hypersensitivity pneumonitis.

85. The method of claim 83, wherein the pneumoconiosis is asbestosis, siliconosis, or Farmer's lung.

86. The method of claim 81, wherein the fibrosis is a fibrotic disease of the liver or lung.

87. The method of claim 86, wherein the fibrotic disease of the lung is caused by rheumatoid arthritis or scleroderma.

88. The method of claim 86, wherein the fibrotic disease of the liver is selected from the group consisting of:
Hepatitis-C;
Hepatitis-B;
cirrhosis;
cirrhosis of the liver secondary to a toxic insult;
cirrhosis of the liver secondary to drugs;
cirrhosis of the liver secondary to a viral infection; and
cirrhosis of the liver secondary to an autoimmune disease.

89. The method of claim 88, wherein the toxic insult is alcohol consumption.

90. The method of claim 88, wherein the viral infection is Hepatitis B, Hepatitis C, or hepatitis non-B non-C.

91. The method of claim 88, wherein the autoimmune disease is primary biliary cirrhosis, or Lupoid hepatitis.

92. A method of treating a condition dependent on CD40 ligand-induced activation of endothelial cells in a subject, comprising the method of claim 38.

93. The method of claim 92, wherein the condition is selected from the group consisting of atherosclerosis, reperfusion injury, allograft rejection, organ rejection, and chronic inflammatory autoimmune diseases.

94. The method of claim 93, wherein the atherosclerosis is accelerated atherosclerosis associated with organ transplantation.

95. The method of claim 93, wherein the chronic inflammatory autoimmune disease is vasculitis, rheumatoid arthritis, scleroderma, or multiple sclerosis.

96. A method of treating a condition dependent on CD40 ligand-induced activation of epithelial cells in a subject, comprising the method of claim 38.

97. The method of claim 96 wherein the epithelial cells are keratinocytes, and the condition is psoriasis.

98. A method of inhibiting activation by CD40 ligand of myeloma cells bearing CD40 on the cell surface, comprising contacting the cells with an agent capable of inhibiting interaction between CD40 ligand and the cells, in an amount effective to inhibit activation of the cells.

99. A method of inhibiting activation by CD40 ligand of myeloma cells bearing CD40 on the cell surface, in a subject, comprising administering to the subject an agent capable of inhibiting interaction between CD40 ligand and the cells, in an amount effective to inhibit activation of the cells in the subject.

100. A method of treating a condition dependent on CD40 ligand-induced activation of myeloma cells in a subject, comprising the method of inhibiting activation by CD40 ligand of myeloma cells bearing CD40 on the cell surface of claim 99.

101. The method of claim 100, wherein the condition is multiple myeloma.
